(19) United States
(12) Patent Application Publication
(10) Pub. No.: US 2024/0343760 A1
(43) Pub. Date: Oct. 17, 2024

(54) PEPTIDES FOR CELL-TYPE SPECIFIC TARGETING OF THE RETINA

(71) Applicants: Oregon State University, Corvallis, OR (US); Oregon Health & Science University, Portland, OR (US)

(72) Inventors: Marco Herrera, Palo Alto, CA (US); Gaurav Sahay, Corvallis, OR (US); Renee Ryals, Portland, OR (US)

(73) Assignees: Oregon State University, Corvallis, US (OR); Oregon Health & Science University, Portland, OR (US)

(21) Appl. No.: 18/742,050

(22) Filed: Jun. 13, 2024

Related U.S. Application Data
(63) Continuation of application No. PCT/US2022/082231, filed on Dec. 22, 2022.
(60) Provisional application No. 63/292,824, filed on Dec. 22, 2021.

(51) Int. Cl. C07K 7/06

(52) U.S. Cl. CPC C07K 7/06

(57) ABSTRACT

Disclosed herein are peptides useful for targeting the retina, and may be useful for targeting specific cells in the retina. The peptides may have a Formula I or V:

R¹—X₁—X₂—(X₃)ₙ—X₄—X₅—X₆—X₇—R² Formula I

R¹-Asn-Val-Ser-Ala-Tyr-Pro-Thr-R² Formula V.

Also disclosed are conjugates and compositions comprising the peptides. The compositions may comprise a desired agent to be delivered to the retina, such as a therapeutic agent and/or an imaging agent. Methods for administering the peptide, conjugates, and/or composition to a subject also are disclosed.

### RELATED APPLICATIONS

This application is a continuation of PCT/US2022/082231, filed Dec. 22, 2022, which claims the benefit under 35 U.S.C. § 119(e) to U.S. Provisional Patent Application No. 63/292,824, filed Dec. 22, 2021, which are incorporated herein by reference.

### ACKNOWLEDGMENT OF GOVERNMENT SUPPORT

This invention was made with government support under 1R21EY031066 awarded by National Institutes of Health. The government has certain rights in the invention.

### FIELD

Disclosed herein are embodiments of a peptide sequence useful for targeting the retina, compositions comprising the peptides, and methods for making and using the peptides and/or compositions thereof.

### REFERENCE TO AN ELECTRONIC SEQUENCE LISTING

The contents of the electronic sequence listing (Sequence_listing.xml; Size: 125,388 bytes; and Date of Creation: Jun. 12, 2024) is herein incorporated by reference in its entirety.

### BACKGROUND

Inherited retinal dystrophies (IRDs) are a group of prevalent genetic disorders that potentially arise from one or more of approximately 250 different gene mutations in the complex biological computer known as the retina. Although these disorders may be mono- or hetero-genic in nature, and one gene may lead to multiple disease phenotypes, they are almost always characterized by their progressively degenerative trend of photoreceptors (PRs) as well as retinal pigmented epithelial cells (RPE) in the back of the neural retina. These disorders can lead to highly impaired vision or full blindness which present patients with tremendous life-altering detriment once diagnosed. Additionally, these genetic complications also affect the clinically-unaffected via their well-established socio-economic burden. Compounding the problem is the reality that most IRDs have long been lacking efficient, cost-effective therapies to restore vision in the over 200 million people afflicted by moderate to severe vision impairment around the globe. Gene therapy to retinal cells is currently and primarily achieved via subretinal injection of viral carriers. Luxturna™, a viral-vectored, biallelic RPE65 protein replacement gene therapy remains the only FDA-approved treatment for inherited retinal dystrophies via subretinal injection. Although encouraging, there still remains a pressing need for an improved delivery method, one that does not present the high risks associated with subretinal injections of viral vectors into already-feeble retinas, while still demonstrating robust cell-specific delivery of diagnostic or therapeutic cargo to PRs and RPE in back of the eye.

### SUMMARY

Disclosed herein are embodiments of a peptide compound useful for targeting the retina. In some embodiments, the peptide has a structure according to Formula I

R¹—X₁—X₂—(X₃)ₙ—X₄—X₅—X₆—X₇—R² Formula I.

With respect to Formula I, n is 0, 1, or 2, such as 1 or 2, and in some embodiments, n is 1.

X₁ is a polar and hydrophilic amino acid, or amphipathic and hydrophobic amino acid, or non-polar and hydrophobic amino acid;

X₂ is a non-polar and hydrophobic amino acid or polar and hydrophilic amino acid;

If present, each X₃ independently is a non-polar and hydrophobic amino acid or polar and hydrophilic amino acid;

X₄ is a non-polar and hydrophobic amino acid or polar and hydrophilic amino acid;

X₅ is a polar and hydrophilic amino acid or non-polar and hydrophobic amino acid;

X₆ is a non-polar and hydrophobic amino acid, or amphipathic and hydrophobic amino acid, or polar and hydrophilic amino acid;

X₇ is a non-polar and hydrophobic amino acid or polar and hydrophilic and basic amino acid; and

R¹ is H, an amino acid, a linker moiety, or a nitrogen protecting group;

R² is OH, an amino acid, a linker moiety, or O(an oxygen protecting group);

and wherein the amino acids are defined as follows:
each hydrophobic amino acid is independently: Ala, Val, Gly, Ile, Leu, Phe, Pro, Trp, Tyr, Met, or Cys;
each hydrophilic amino acid is independently: Arg, Asn, Asp, Glu, Gln, Lys, Ser, Thr, or His;
each non-polar amino acid is independently: Ala, Gly, Ile, Leu, Phe, Val, or Pro; each amphipathic amino acid is independently: Trp, Tyr, or Met;
each polar amino acid is independently: Ser, Thr, Asn, Gln, Arg, Lys, Asp, His, Cys, or Glu;
each basic amino acid is independently: Arg, Lys, or His; and each acidic amino acid is independently: Asp or Glu.

X₁ is polar/hydrophilic or amphipathic/hydrophobic or non-polar/hydrophobic. X₂ is non-polar/hydrophobic or polar/hydrophilic. If present, each X₃ independently is non-polar/hydrophobic or polar/hydrophilic. X₄ is non-polar/hydrophobic or polar/hydrophilic. X₅ is polar/hydrophilic or non-polar/hydrophobic. X₆ is non-polar/hydrophobic or amphipathic/hydrophobic or polar/hydrophilic. X₇ is non-polar/hydrophobic or polar/hydrophilic/basic. In certain embodiments, R¹ is H, an amino acid residue, or a linker moiety; and R² is OH, an amino acid residue, or a linker moiety.

In some embodiments, X₂ and/or X₇ may be substituted with a Cys residue.

In some embodiments, one or more of the following conditions apply:
a) the peptide contains one or more polar amino acid residues;
b) the peptide contains at least one hydrophobic amino acid residue;
c) the peptide contains 1-3 hydrophobic amino acid residues;
d) the peptide contains 1-5 non-polar amino acid residues;
e) the peptide does not contain an acid amino acid residue.

In certain embodiments, X₁ is a non-polar and hydrophobic amino acid, or Ser, or Met; X₂ is a non-polar and hydrophobic amino acid, or Asn, or Thr; each X₃ independently is a non-polar and hydrophobic amino acid or Ser when n is 1 or 2; X₄ is a non-polar and hydrophobic amino acid or Ser; X₅ is a non-polar and hydrophobic amino acid, or polar and hydrophobic and basic amino acid, such as non-polar and hydrophobic amino acid, or Arg, or His; X₆ is a non-polar and hydrophobic amino acid, or polar and hydrophobic amino acid, or Tyr; X₇ is a non-polar and hydrophobic amino acid or Arg; or a combination thereof.

In certain embodiments, X₁ is a non-polar and hydrophobic amino acid, or Ser, or Met; X₂ is a non-polar and hydrophobic amino acid, or Asn, or Thr; each X₃ independently is a non-polar and hydrophobic amino acid or Ser when n is 1 or 2; X₄ is a non-polar and hydrophobic amino acid or Ser; X₅ is a non-polar and hydrophobic amino acid, or polar and hydrophobic and basic amino acid, such as non-polar and hydrophobic amino acid, or Arg, or His; X₆ is a non-polar and hydrophobic amino acid, or polar and hydrophobic amino acid, or Tyr; or X₇ is a non-polar and hydrophobic amino acid or Arg; or a combination thereof.

In certain embodiments, X₁ is a polar and hydrophilic amino acid, X₂ is a non-polar and hydrophobic amino acid, X₃ is a non-polar and hydrophobic amino acid, X₄ is a non-polar and hydrophobic amino acid, X₅ is a polar and hydrophilic and basic amino acid, X₆ is a non-polar and hydrophobic amino acid, and X₇ is a non-polar and hydrophobic amino acid; or

X₁ is a polar and hydrophilic amino acid, X₂ is a polar and hydrophilic amino acid, X₃ is a non-polar and hydrophobic amino acid, X₄ is a non-polar and hydrophobic amino acid, X₅ is a non-polar and hydrophobic amino acid, X₆ is a non-polar and hydrophobic amino acid, and X₇ is a non-polar and hydrophobic amino acid; or

X₁ is an amphipatic and hydrophobic amino acid, X₂ is a non-polar and hydrophobic amino acid, X₃ is a non-polar and hydrophobic amino acid, X₄ is a non-polar and hydrophobic amino acid, X₅ is a non-polar and hydrophobic amino acid, X₆ is an amphipatic and hydrophobic amino acid, and X₇ is a polar and hydrophilic and basic amino acid; or

X₁ is a non-polar and hydrophobic amino acid, X₂ is a polar and hydrophilic amino acid, X₃ is a non-polar and hydrophobic amino acid, X₄ is a non-polar and hydrophobic amino acid, X₅ is a polar and hydrophilic and basic amino acid, X₆ is a polar and hydrophilic amino acid, and X₇ is a non-polar and hydrophobic amino acid; or

X₁ is a polar and hydrophilic and basic amino acid, X₂ is a non-polar and hydrophobic amino acid, X₃ is a polar and hydrophilic amino acid, X₄ is a polar and hydrophilic amino acid, X₅ is a non-polar and hydrophobic amino acid, X₆ is a polar and hydrophilic amino acid, and X₇ is a non-polar and hydrophobic amino acid.

In particular embodiments, X₁ is Ser, Met, Ala or His; X₂ is Pro, Asn, Thr or Leu; If present, each X₃ independently is Ala, Leu, Val, Gly, or Ser; X₄ is Leu, Ala, Pro, or Ser; X₅ is His, Ala, Val, Arg, or Leu; X₆ is Phe, Tyr, Ser, or Thr; X₇ is Leu, Pro, Arg, Val, or Pro; or a combination thereof.

In some embodiments, R¹ is H, R² is OH, and/or the peptide is selected from:

Peptide 42:
(SEQ ID No. 23)
Ser-Pro-Ala-Leu-His-Phe-Leu;
Peptide 43:
(SEQ ID No. 24)
Ser-Asn-Leu-Ala-Ala-Phe-Pro;
Peptide 50:
(SEQ ID No. 26)
Met-Pro-Val-Ala-Val-Tyr-Arg;
Peptide 57:
(SEQ ID No. 29)
Ala-Thr-Gly-Pro-Arg-Ser-Val;
or
Peptide 54:
(SEQ ID NO. 28)
His-Leu-Ser-Ser-Leu-Thr-Pro.

In other embodiments, at least one of R¹ and R² is a linker moiety, and in some embodiments, R¹ is H and R² is the linker moiety. In any embodiments, the linker moiety may be a peptide sequence having from 2 to 7 amino acid residues, such as 5 amino acid residues. In certain embodiments, the linker moiety is a peptide sequence having 4 amino acids. And in some embodiments, the linker moiety has a sequence Gly-Gly-Gly-Ser (GGGS; SEQ ID No. 35), Gly-Gly-Gly-Ser-Cys (GGGSC; SEQ ID No. 52) or Gly-Gly-Gly-Ser-Lys (GGGSK; SEQ ID No. 53). In some embodiments, the peptide may be selected from:

(SEQ ID No. 32)
Ser-Pro-Ala-Leu-His-Phe-Leu-Gly-Gly-Gly-Ser-Cys;
(SEQ ID No. 33)
Ser-Asn-Leu-Ala-Ala-Phe-Pro-Gly-Gly-Gly-Ser-Cys;
or
(SEQ ID No. 34)
Met-Pro-Val-Ala-Val-Tyr-Arg-Gly-Gly-Gly-Ser-Cys.

In some embodiments, the peptide is a peptide shown in Table 6. In alternative embodiments, one or more of, such as both of, R¹ and R² is an amino acid residue. In some embodiments, R¹ and R² are each Cys and/or the peptide may have a structure according to Formula III or Formula IV

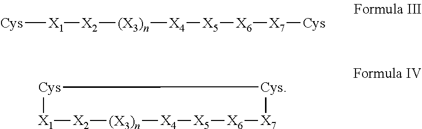

In another aspect, the present disclosure provides conjugates of the formula:

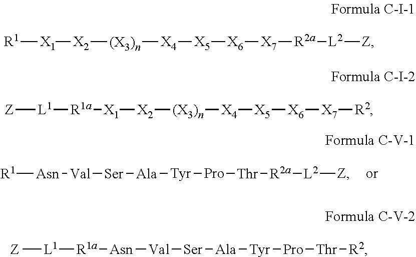

wherein:
R¹, X₁, X₂, X₃, n, X₄, X₅, X₆, X₇, and R² are as defined herein;
R^{1a} is a divalent radical formed by removing one hydrogen atom from R¹;
R^{2a} is a divalent radical formed by removing one hydrogen atom from R²;
each of L¹ and L² is a second linker moiety; and
Z is a lipid, small molecule, peptide, polypeptide, protein, nucleic acid, or saccharide.

Also disclosed herein are embodiments, of compositions comprising the disclosed peptide and an additional agent. In certain embodiments, the additional agent is a pharmaceutical agent. The additional agent may be a lipid nanoparticle or an imaging agent, such as a dye, fluorophore or radiotracer. The lipid nanoparticle may further comprise or contain a therapeutic agent, such as a nucleic acid, for example, an antisense oligonucleotide (ASO), mRNA, siRNA, miRNA, sgRNA/pegRNA, or a combination thereof. Additionally, or alternatively, the composition may comprise pharmaceutically acceptable excipient.

In another aspect, the present disclosure provides methods of making the composition, the methods comprise:
providing a first solution comprising the peptide or conjugate;
providing a second solution; and
mixing the first and second solutions to form a mixture.

Embodiments of a method for using the disclosed peptide and/or a composition thereof also is disclosed. The method may comprise administering to a subject a peptide according to the present disclosure or a composition thereof. Administering to the subject may comprise administering to an eye of the subject, and/or may comprise administering by injection, such as by intravitreal or subretinal injection.

The moiety —X₁—X₂—(X₃)ₙ—X₄—X₅—X₆—X₇—may be able to bind to a PR, RPE, and/or Müller glia. The linker moiety of R¹ and/or R² may comprise a reaction handle (e.g., —SH). The reaction handle may react with an orthogonal reaction handle (e.g., a thiophile (e.g., a Michael acceptor or activated ester)) of a lipid, small molecule, peptide, polypeptide, protein, nucleic acid, or saccharide to form a conjugate.

The conjugate may comprise an additional agent. The additional agent may be covalently attached to the rest of the conjugate. The conjugate may be administered to the eye of a subject and deliver the additional agent to the eye (e.g., the retina). The delivery may be at least in part due to the binding of —X₁—X₂—(X₃)ₙ—X₄—X₅—X₆—X₇—to the PR, RPE, and/or Müller glia.

The conjugate may comprise no additional agents. The conjugate may comprise a lipid (e.g., an ionizable lipid (e.g., phospholipid)). The conjugate may be a component of a composition. The composition may further comprise an additional lipid (e.g., an ionizable lipid (e.g., phospholipid), a polymer-conjugated lipid (e.g., a PEG-(fatty acid diglyceride)), and/or a structural lipid (e.g., a sterol)). The composition may further comprise an additional agent. The additional agent may be non-covalently associated with the rest of the composition (e.g., conjugate). The composition may further comprise a particle (e.g., nanoparticle) and/or micelle. The particle or micelle may comprise the conjugate, additional lipid, and additional agent. The conjugate and additional lipid may be part of the outer shell of the particle or micelle. The additional agent may be encapsulated in the particle or micelle. The composition may be administered to the eye of a subject and deliver the additional agent to the eye (e.g., the retina). The delivery may be at least in part due to the binding of —X₁—X₂—(X₃)ₙ—X₄—X₅—X₆—X₇—to the PR, RPE, and/or Müller glia.

The foregoing and other objects, features, and advantages of the invention will become more apparent from the following detailed description, which proceeds with reference to the accompanying figures.

### BRIEF DESCRIPTION OF THE DRAWINGS

FIGS. 1A to 1I show in vivo development of peptide-phage display biopanning FIG. 1A depicts an in vivo schematic. Heptameric phage libraries were injected intravitreally into BALB/c mice and incubated for 6 hrs followed by retina extraction. Loosely-bound phages were subjected to washes and elution to be used in titering and amplification rounds for subsequent biopanning rounds. The process was carried out three times and enriched specific phage-peptide binders were isolated, followed by DNA extraction and Sanger sequencing. FIG. 1B shows in vivo phage titers after each round of biopanning. FIG. 1C shows in vivo peptide-targeting validation. Empty M13 Bacteriophage were used as a negative control compared to peptide-containing library at the same concentration injected intravitreally in mice using an ordinary one-way ANOVA. n=3; mean±SEM. ***p<0.001. FIGS. 1D to 1F are heatmaps depicting phage biopanning enrichment after rounds 1-3 in vivo of peptide sequences isolated for each round. For biopanning rounds, n=3 with at least 5 technical replicates. FIGS. 1G to 1I show the consensus sequence for each amino acid in the heptameric sequence isolated after biopanning rounds 1-3 in vivo.

FIGS. 2A to 2H shows in vitro binding, structural analysis and internalization of in vivo-isolated peptide candidates. Single phage-peptide differential binding properties were elucidated using cell-based ELISA against model cell lines of desired target tissues in neural retina. Three high-performing hits, as well as a low binder were selected from ELISA results for further analysis and validation across both cell lines. FIGS. 2A to 2B show cell-ELISA results for ARPE19 cells and 661w cells, respectively, with selected hits being: FIG. 2A: 50, 42, 3, and 43; FIG. 2B: 42, 50, 43, and 3. Empty M13-Bacteriophage used as negative control. FIGS. 2C to 2D show molecular operating environment (MOE) structural superposition of selected candidates for ARPE19 and 661w cells, respectively. FIGS. 2E and 2G show confocal microscopy images of TAMRA-labelled peptides 42 and 50 cell-internalization after 30-minute incubation with ARPE19 and 661w cells, respectively. Scale bar represents 50 μm. FIGS. 2F and 2H show mean fluorescence intensity quantification of confocal images. ELISA binding experiments performed in duplicate with 6 technical replicates; mean±SEM. An ordinary one-way ANOVA, with Tukey's correction for multiple comparisons test was used for comparisons between treatments. *p≤0.05, **p≤0.01, ****p<0.0001.

FIGS. 3A to 3O show in vivo injections of TAMRA-conjugated peptide candidates in BALB/c mice. Representative images of the clearance kinetics and in vivo targeting of TAMRA-labelled peptides injected intravitreally and subretinally into BALB/c mice and extracted at specific time points post-injection. FIGS. 3A to 3D show in vivo fundus images of peptide MH42 intravitreally and subretinally delivered at the corresponding timepoints. Top panel displays brightfield images of the eye, and bottom shows fluorescence demonstrating localization of the peptide. FIGS. 3E to 3L are confocal images of 12 μm cryo-sections following intravitreal and subretinal injections of peptides MH42 and MH50, respectively, to validate peptide targeting. Scale bar represents 25 μm. FIG. 3M is a schematic identifying the RPE/choroid and PR layers used for quantification. For the analysis, these layers were manually segmented in ImageJ. FIG. 3N shows intravitreal and FIG. 3O shows subretinal MFI quantification of confocal images for localized fluorescence in PR or RPE/choroid layers. An ordinary one-way ANOVA, with Tukey's correction for multiple comparisons test was used for comparisons between groups. n=4-8 eyes per group; mean±SEM. *p≤0.05, **p≤0.01, ***p≤0.001, ****p≤0.0001. TAMRA-dye injected as negative control. GCL—ganglion cell layer, INL—inner nuclear layer, ONL—outer nuclear layer, RPE—retinal pigment epithelium.

FIGS. 4A to 41 show peptide-conjugated LNP characterization and in vivo injections in Ai9-tdTomato mice with increasing peptide surface density. Particle characterization and representative confocal images of MH42 peptide-conjugated, Cre-recombinase mRNA-loaded LNPs intravitreally administered to Ai6 Rosa mice. FIG. 4A is a schematic of LNP formulation and conjugation with peptide via maleimide-thiol chemistry, and Cre mouse model depicting both routes of administration trialed. FIGS. 4B to 4C are graphs depicting size, polydispersity, and mRNA encapsulation efficiency of the LNPs with varying ratios of MH42 peptide conjugation. % refers to molar percent of peptide-functionalized PEG in the formulation. FIGS. 4D to 4F are cryo-TEM images of the unconjugated control LNP, 0.15%-MH42, and 0.3%-MH42 peptide-LNP. Scale bar represents 20 nm. FIG. 4G are representative fundus images showing in vivo tdTomato expression after intravitreal delivery of unconjugated and conjugated LNPs. FIG. 4H show confocal microscopy images of tdTomato expression following intravitreal uptake and translation of Cre mRNA LNPs. FIG. 4I show confocal images showing no T cell infiltration (CD3) or microglia activation (IBA-1) associated with intravitreal delivery and expression. Microglia were restricted to the plexiform layers (arrows). Confocal images taken at 20×. n=6 eyes per group. Scale bar represents 50 μm. GCL-ganglion cell layer, INL—inner nuclear layer, ONL-outer nuclear layer.

FIGS. 5A to 5D show MH42 conjugated LNPs mediate PR expression after subretinal administration. Representative fundus images showing in vivo tdTomato expression combined with 40× confocal images of retinal cross sections expressing tdTomato and stained with visual arrestin (e.g., rods and cones) and DAPI for FIG. 5A PBS, FIG. 5B untargeted LNPs (n=2), FIG. 5C 0.15% MH42 LNPs (n=6) and FIG. 5D 0.3% MH42 LNPs (n=4). GCL—ganglion cell layer, INL-inner nuclear layer, ONL-outer nuclear layer, RPE—retinal pigment epithelium.

FIGS. 6A to 6H show retinal toxicity associated with subretinal administration of MH42-LNPs. FIGS. 6C and 6D are representative images from retinas injected with 0.15% MH42-LNPs (n=6), while FIGS. 6G and 6H are representative images from retinas injected with 0.3% MH42-LNPs (n=4). FIGS. 6A and 6E are confocal images at 10× containing tdTomato expression and labeled with DAPI. FIGS. 6B and 6F and FIGS. 6H and 6E are images showing the retinal morphology in some areas of tdTomato expression. (C & G) Confocal images at 40× demonstrating tdTomato expression and stained with visual arrestin and DAPI. FIGS. 6D and 6H are confocal images at 40× showing tdTomato expression and stained with CD3 (T cells), IBA-1 (microglia) and DAPI. GCL—ganglion cell layer, INL—inner nuclear layer, ONL—outer nuclear layer, RPE—retinal pigment epithelium.

FIGS. 7A to 7G show MH42 conjugated LNPs mediate expression in the neural retina after subretinal administration in the NHP. FIG. 7A show wide field fundus autofluorescence imaging 48 hours-post subretinal delivery of MH42-LNPs (n=1). Circle demarks the location of the bleb, and the horizontal line indicates cross section that corresponds to IHC. FIG. 7B shows montaged 10× confocal images of primate retinal cross-sections labeled with anti-GFP antibody and DAPI. A montaged 10× H & E image shows the retinal morphology in areas of GFP expression. FIG. 7C to 7F are 40× confocal images of retinal cross-sections co-stained with anti-GFP and cell specific antibodies cone arrestin (cones), rod arrestin (rods and s-cones), RPE65 (RPE) and glutamine synthetase (Miller glia). FIG. 7G shows 20× confocal cross-sections labeled with CD3 (T cells) and IBA-1 (microglia) to observe the elicited immune response. GltS—glutamine synthetase, GFP—green fluorescent protein, GCL—ganglion cell layer, INL—inner nuclear layer, ONL—outer nuclear layer, RPE—retinal pigment epithelium.

FIGS. 8A to 8B show cell uptake of TAMRA-labelled peptide candidates. Confocal microscopy images of hARPE19 in FIG. 8A and 661w cone cells in FIG. 8B incubated with 10 nM and 50 nM peptides 3 and MH43.

TAMRA Dye Used as Negative Control. Scale Bars Represent 50 μm

FIGS. 9A to 9D are TAMRA-tagged peptide uptake in BALB/c mice. FIG. 9A shows representative confocal microscopy images of BALB/c mice eyes intravitreally injected with peptides MH3, MH43, MH42 and MH50 collected at 1 hr and 24 hrs. FIG. 9B shows representative confocal images of TAMRA-MH3 and -MH43 delivered intravitreally and subretinally. Scale bar represents 25 μm. FIGS. 9C to 9D show mean fluorescence intensity fold-change of peptide uptake in vivo of MH42 and MH50 over TAMRA control; n=4-8 eyes per group.

FIG. 10 shows confocal microscopy images of PBS injected control as well as 0.6-1.2% formulations tested. Scale bar represents 50 μm. Functionalized PEG component in LNP formulations with different peptide densities are shown in Table 11.

FIG. 11 is a graph of PFU versus biopanning rounds illustrating the in vitro bacteriophage titers after 3 rounds of biopanning against hARPE19 cells

FIG. 12 is a graph of PFU versus biopanning rounds illustrating the in vitro bacteriophage titers after 3 rounds of biopanning against 661w cone cells.

FIG. 13 are heatmaps illustrating the amino acid occurrences at each position after each round of 3 rounds of biopanning in balb/c mice.

FIG. 14 are digital 40× confocal microscopy images illustrating the cell uptake of hARPE19 cells incubated with 10 nM and 50 nM of each of the TAMRA control, MH3, MH42, MH43 and MH50 peptides.

FIG. 15 are digital 40× confocal microscopy images illustrating the cell uptake of 661w cone cells incubated with 10 nM and 50 nM of each of the TAMRA control, MH3, MH42, MH43 and MH50 peptides.

FIG. 16 are digital images illustrating the location of MH42 TAMRA-conjugated peptide at various timepoints when administered in vivo intravitreally and subretinally.

FIG. 17 are digital images illustrating the location of MH50 TAMRA-conjugated peptide at various timepoints when administered in vivo intravitreally and subretinally.

FIG. 18 are digital images illustrating the location of MH3 TAMRA-conjugated peptide at various timepoints when administered in vivo intravitreally and subretinally.

FIG. 19 are digital images illustrating the location of MH43 TAMRA-conjugated peptide at various timepoints when administered in vivo intravitreally and subretinally.

FIG. 20 is a graph of particle size versus formulation, illustrating the particle size of the lipid nanoparticle-peptide conjugates (LNP-P) at different levels of substitution.

FIG. 21 is a graph of polydispersity index versus formulation, illustrating the polydispersity index of the LNP-P at different levels of substitution.

FIG. 22 is a graph of RNA encapsulation versus formulation, illustrating the RNA encapsulation of the LNP-P at different levels of substitution.

FIG. 23 are digital confocal microscopy images of peptide-conjugated, Cre-recombinase mRNA-loaded LNPs intravitreally administered to Ai-9 tdTomato mice to validate targeted delivery to neural retina.

FIG. 24 are digital confocal microscopy images of peptide-conjugated, Cre-recombinase mRNA-loaded LNPs subretinally administered to Ai-9 tdTomato mice to validate targeted delivery to neural retina.

FIGS. 25A to 25B show confocal images of rhesus tonsils. FIG. 25A is a confocal image of rhesus tonsil stained with IBA-1 (microglia) and DAPI. FIG. 25B is a confocal image of rhesus tonsil stained with CD3 (T cells) and DAPI. Slides were stained alongside retinal cross-sections to serve as positive controls.

### SEQUENCE LISTING

The nucleic and amino acid sequences listed in the accompanying sequence listing are shown using standard letter abbreviations for nucleotide bases, and three letter code for amino acids, as defined in 37 C.F.R. § 1.822. Only one strand of each nucleic acid sequence is shown, but the complementary strand is understood as included by any reference to the displayed strand.

SEQ ID Nos. 1-30 are the peptide sequences screened in the peptide phage library.

SEQ ID Nos. 31-34 are exemplary peptide sequences with the amino acid linker moiety attached.

SEQ ID No. 35 is the peptide sequence of an exemplary amino acid linker moiety.

SEQ ID No. 36 is the nucleic acid sequence of the primer used for the biopanning.

SEQ ID No. 37 is an amino acid sequence motif identified after the three rounds of phage display panning.

SEQ ID Nos. 38-51 are peptide sequences identified in three rounds of in vivo biopanning in balb/c mice.

SEQ ID Nos. 52-53 are the peptide sequences of additional exemplary amino acid linker moieties.

SEQ ID Nos. 54-98 are additional exemplary peptide sequences suitable for use in the disclosed technology.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “alkyl” refers to a radical of a C₁-C₁₀₀₀ straight-chain or branched saturated hydrocarbon group. In some embodiments, an alkyl group has 3 to 400 carbon atoms (“C₃-C₄₀₀ alkyl”), 20 to 200 carbon atoms (“C₂₀-C₂₀₀ alkyl”), 1 to 20 carbon atoms (“C₁-C₂₀ alkyl”), 1 to 10 carbon atoms (“C₁-C₁₀ alkyl”), 1 to 9 carbon atoms (“C₁-C₉ alkyl”), 1 to 8 carbon atoms (“C₁-C₈ alkyl”), 1 to 7 carbon atoms (“C₁-C₇ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), 1 to 2 carbon atoms (“C₁-C₂ alkyl”), or 1 carbon atom (“C₁ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C), tertiary amyl (C), and n-hexyl (C₆). Additional examples of alkyl groups include n-heptyl (C₇), n-octyl (C₈) and the like. C₃₀-C₁₀₀₀ alkyl may be obtained from polymerization. Unless otherwise specified, each instance of an alkyl group is independently unsubstituted (an “unsubstituted alkyl”) or substituted (a “substituted alkyl”) with one or more substituents.

The term “alkenyl” refers to a radical of a straight-chain or branched hydrocarbon group having from 2 to 1000 carbon atoms and one or more carbon-carbon double bonds (e.g., 1, 2, 3, or 4 double bonds). In some embodiments, an alkenyl group has 2 to 400 carbon atoms (“C₂₋₄₀₀ alkenyl”) or 20 to 200 carbon atoms (“C₂₀-C₂₀₀ alkenyl”). In some embodiments, an alkenyl group has 2 to 20 carbon atoms (“C₂₋₂₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. C₃₀-C₁₀₀₀ alkenyl may be obtained from polymerization. Unless otherwise specified, each instance of an alkenyl group is independently unsubstituted (an “unsubstituted alkenyl”) or substituted (a “substituted alkenyl”) with one or more substituents. In an alkenyl group, a C═C double bond for which the stereochemistry is not specified

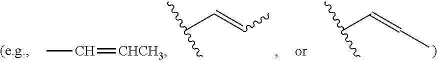

may be in the (E)- or (Z)-configuration.

The term “alkynyl” refers to a radical of a straight-chain or branched hydrocarbon group having from 2 to 1000 carbon atoms and one or more carbon-carbon triple bonds (e.g., 1, 2, 3, or 4 triple bonds) (“C₂₋₁₀ alkynyl”). In some embodiments, an alkynyl group has 2 to 400 carbon atoms (“C₂₋₄₀₀ alkynyl”), 20 to 200 carbon atoms (“C₂₀-C₂₀₀ alkynyl”), 2 to 20 carbon atoms (“C₂₋₂₀ alkynyl”), 2 to 9 carbon atoms (“C₂₋₉ alkynyl”), 2 to 8 carbon atoms (“C₂₋₈ alkynyl”), 2 to 7 carbon atoms (“C₂₋₇ alkynyl”), 2 to 6 carbon atoms (“C₂₋₆ alkynyl”), 2 to 5 carbon atoms (“C₂₋₅ alkynyl”), 2 to 4 carbon atoms (“C₂₋₄ alkynyl”), 2 to 3 carbon atoms (“C₂₋₃ alkynyl”), or 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. C₃₀-C₁₀₀₀ alkynyl may be obtained from polymerization. Unless otherwise specified, each instance of an alkynyl group is independently unsubstituted (an “unsubstituted alkynyl”) or substituted (a “substituted alkynyl”) with one or more substituents.

The term “heteroalkyl” refers to an alkyl group which further includes at least one heteroatom (e.g., 1, 2, 3, 4, or more heteroatoms, as valency permits) selected from oxygen, nitrogen, phosphorus, or sulfur within (i.e., inserted between adjacent carbon atoms of) and/or placed at one or more terminal position(s) of the parent chain. In certain embodiments, a heteroalkyl group refers to a saturated group having from 1 to 1000 carbon atoms and 1 or more heteroatoms within the parent chain (“C₁-C₁₀₀₀ heteroalkyl” or “C₁₋₁₀₀₀ heteroalkyl”), 2 to 400 carbon atoms and 1 or more heteroatoms within the parent chain (“C₂-C₄₀₀ heteroalkyl”), 1 to 20 carbon atoms and 1 or more heteroatoms within the parent chain (“C₁-C₂₀ heteroalkyl”), 1 to 10 carbon atoms and 1 or more heteroatoms within the parent chain (“C₁-C₁₀ heteroalkyl”), 1 to 9 carbon atoms and 1 or more heteroatoms within the parent chain (“C₁-C₉ heteroalkyl”), 1 to 8 carbon atoms and 1 or more heteroatoms within the parent chain (“C₁-C₈ heteroalkyl”), 1 to 7 carbon atoms and 1 or more heteroatoms within the parent chain (“C₁-C₇ heteroalkyl”), 1 to 6 carbon atoms and 1 or more heteroatoms within the parent chain (“C₁-C₆ heteroalkyl”), 1 to 5 carbon atoms and 1 or more heteroatoms within the parent chain (“C₁-C₅ heteroalkyl”), 1 to 4 carbon atoms and 1 or more heteroatoms within the parent chain (“C₁-C₄ heteroalkyl”), 1 to 3 carbon atoms and 1 or more heteroatoms within the parent chain (“C₁-C₃ heteroalkyl”), 1 to 2 carbon atoms and 1 heteroatom within the parent chain (“C₁-C₂ heteroalkyl”), or 1 carbon atom and 1 heteroatom (“C₁ heteroalkyl”). C₃₀-C₁₀₀₀ heteroalkyl may be obtained from polymerization. Unless otherwise specified, each instance of a heteroalkyl group is independently unsubstituted (an “unsubstituted heteroalkyl”) or substituted (a “substituted heteroalkyl”) with one or more substituents.

The term “heteroalkenyl” refers to an alkenyl group, which further includes at least one heteroatom (e.g., 1, 2, 3, 4, or more heteroatoms, as valency permits) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 1000 carbon atoms, at least one double bond, and 1 or more heteroatoms within the parent chain (“heteroC₂₋₁₀₀₀ alkenyl” or “C₂₋₁₀₀₀ heteroalkenyl”), or 40 to 400 carbon atoms, at least one double bond, and 1 or more heteroatoms within the parent chain (“heteroC₄₋₄₀₀ alkenyl”). In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). C₃₀-C₁₀₀₀ heteroalkenyl may be obtained from polymerization. Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl” refers to an alkynyl group, which further includes at least one heteroatom (e.g., 1, 2, 3, 4, or more heteroatoms, as valency permits) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 1000 carbon atoms, at least one triple bond, and 1 or more heteroatoms within the parent chain (“heteroC₂₋₁₀₀₀ alkynyl” or “C₂₋₁₀₀₀ heteroalkynyl”), or 40 to 400 carbon atoms, at least one double bond, and 1 or more heteroatoms within the parent chain (“heteroC₄₀₋₄₀₀ alkynyl”). In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂-s alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). C₃₀-C₁₀₀₀ heteroalkynyl may be obtained from polymerization. Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” or “cycloalkyl” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”), 3 to 7 ring carbon atoms (“C₃₋₇ carbocyclyl”), 3 to 6 ring carbon atoms (“C₃₋₆ carbocyclyl”), 4 to 6 ring carbon atoms (“C₄₋₆ carbocyclyl”), 5 to 6 ring carbon atoms (“C₅₋₆ carbocyclyl”), or 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃-s carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, phosphorus, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents.

In some embodiments, a heterocyclyl group is a 5-10 membered non-aromatic ring system having ring carbon atoms and 1-4 ring heteroatoms, wherein each heteroatom is independently selected from nitrogen, oxygen, phosphorus, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, phosphorus, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, phosphorus, and sulfur (“5-6 membered heterocyclyl”). In some embodiments, the 5-6 membered heterocyclyl has 1-3 ring heteroatoms selected from nitrogen, oxygen, phosphorus, and sulfur. In some embodiments, the 5-6 membered heterocyclyl has 1-2 ring heteroatoms selected from nitrogen, oxygen, phosphorus, and sulfur. In some embodiments, the 5-6 membered heterocyclyl has 1 ring heteroatom selected from nitrogen, oxygen, phosphorus, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl, and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydro-benzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group be monovalent or may have more than one point of attachment to another moiety (e.g., it may be divalent, trivalent, etc), although the valency may be specified directly in the name of the group. For example, “triazoldiyl” refers to a divalent triazolyl moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As understood from the above, alkyl, alkenyl, alkynyl, carbocyclyl, aryl, and heteroaryl groups are, in certain embodiments, optionally substituted. Optionally substituted refers to a group which may be substituted or unsubstituted (e.g., “substituted” or “unsubstituted” alkyl). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Affixing the suffix “ene” to a group indicates the group is a polyvalent (e.g., bivalent, trivalent, tetravalent, or pentavalent) moiety. In certain embodiments, affixing the suffix “ene” to a group indicates the group is a bivalent moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR—, —SSR^{cc}, —C(═O)R^{aa}, —CO₂H, —CHO, —C(OR^{cc})₂, —CO₂R^{aa}, —OC(═O)R^{a}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc}) —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NRC(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁-6 perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; and
each X⁻ is a counterion.

In certain embodiments, the carbon atom substituents are independently halogen, substituted or unsubstituted, C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted or unsubstituted, C₁₋₆ alkyl, —OR^{aa}, —SR^{aa}, —N(R^{bb})₂, —CN, —SCN, or —NO₂.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{bb})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂-10 alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonoethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH.

The term “thiol” or “thio” refers to the group —SH.

The term “amine” or “amino” refers to the group —NH— or —NH₂.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate (triflate), p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes. In certain embodiments, the counterion is triflate.

The term “leaving group” refers to an atom or a group capable of being displaced by a nucleophile. Examples of suitable leaving groups include halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other examples of leaving groups are water, ammonia, alcohols, ether moieties, thioether moieties, zinc halides, magnesium moieties, diazonium salts, and copper moieties.

“Click chemistry” reaction includes Huisgen alkyne-azide cycloaddition. Any “click chemistry” reaction known in the art can be used to this end. Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition).

“Small Molecule” refers to an organic molecule having a molecular weight of about 2000 Daltons or less. In some embodiments, the term “small molecule” refers to a compound that is not a polypeptide, protein, or nucleic acid molecule. A small molecule may be a small molecule therapeutic and/or prophylactic, such as an antibiotic, anti-inflammatory, anticancer, antiviral, immunosuppressant, analgesic, antifungal, antiparasitic, anticonvulsants, antidepressant, anti-anxiety, anti-psychotic, and the like. In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 g/mol, not more than 900 g/mol, not more than 800 g/mol, not more than 700 g/mol, not more than 600 g/mol, not more than 500 g/mol, not more than 400 g/mol, or not more than 300 g/mol. In certain embodiments, the molecular weight of a small molecule is between 200 and 300 g/mol, between 300 and 400 g/mol, between 400 and 600 g/mol, between 600 and 800 g/mol, between 800 and 1,000 g/mol, between 1,000 and 1,300 g/mol, between 1,300 and 1,600 g/mol, or between 1,600 and 2,000 g/mol,

The term “polymer” refers to a compound comprising eleven or more covalently connected repeating units. In certain embodiments, a polymer is naturally occurring. In certain embodiments, a polymer is synthetic (e.g., not naturally occurring). In certain embodiments, the number average molecular weight of the polymer (e.g., as determined by gel permeation chromatography) is between 1,000 and 3,000, between 3,000 and 10,000, between 10,000 and 30,000, between 30,000 and 100,000, between 100,000 and 300,000, or between 300,000 and 1,000,000, g/mol, inclusive. In certain embodiments, the dispersity of the polymer is between 1 and 1.2, between 1.2 and 1.5, between 1.5 and 2, between 2 and 4, between 4 and 10, inclusive.

“Lipid” refers to an organic compound that is readily soluble in nonpolar solvents such as hydrocarbons, but typically is sparingly or non-soluble in water, and may be poorly soluble in other polar solvents. Ionizable lipids are lipids that can be ionized, for example, with pH-dependent ionization. The lipid may be anionic and/or cationic, for example, it may form an anion and/or a cation depending on pH. In some embodiments, an ionizable lipid may be positive at low pH, and may be substantially neutral at physiological or neutral pH.

A “basic nitrogen atom” refers to the nitrogen atom to which R and R′ are attached in an amino moiety, wherein the nitrogen atom is not attached to any one of —C(O)—, —S(O)—, —S(O)₂—, —P(O), —P(O)₂, and

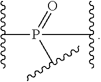 [SMILES: *P(*)(*)=O]

In certain embodiments, R and R′ together with the nitrogen atom to which they are attached do not form a heteroaryl ring.

“Nanoparticle” as used herein refers to a composition, such as a pharmaceutical formulation, having a particle size (for example, a diameter) of from 1 to 1000 nanometers, such as from 1 to 500 nanometers or from 1 to 100 nanometers, and incorporating one or more lipid compounds disclosed herein. In certain embodiments, the average (e.g., mean) dimension (e.g., diameter or length) of a nanoparticle is between 1 and 3, between 3 and 10, between 10 and 30, between 30 and 100, between 100 and 300, or between 300 and 1,000, nm, inclusive. In certain embodiments, the average dimension is determined with dynamic light scattering. Nanoparticle compositions include, but are not limited to, lipid nanoparticles (LNPs), liposomes (e.g., lipid vesicles), and lipoplexes.

“Microparticle” refers to a particle having an average (e.g., mean) dimension (e.g., diameter or length) of between 1 and 3, between 3 and 10, between 10 and 30, between 30 and 100, between 100 and 300, or between 300 and 1,000, μm, inclusive. In certain embodiments, the average dimension is determined with dynamic light scattering.

“Lipid nanoparticle” (LNP) refers to a nanoparticle comprising one or more lipid compounds. Typically, the lipid compound(s) will be a major component of the nanoparticle. LNPs may be substantially spherical in shape. Disclosed LNPs may be positively charged in low pH and substantially neutral at physiological pH. Alternatively, the LNP may be uncharged, even if the lipids themselves are charged. In some embodiments, the ionizable lipid is contained in the core and its charge may be shielded by other lipid components. LNPs that can be used to deliver mRNA, siRNA and genome editing components

“Nucleic acid” refers to a polynucleotide molecule. The polynucleotide may be a naturally occurring polynucleotide or a synthetic polynucleotide. A nucleic acid may be a DNA, RNA or mixture of DNA and RNA nucleotides. Typically, the nucleic acid contains from 20 to 10,000 nucleotides or more, such as from 100, 200, 300, 400, 500, 600, 700, 800, 900, 1000, 2000, 3000, 4000, or 5000 nucleotides to 10,000 nucleotides.

Exemplary nucleic acids include, but are not limited to, single stranded DNA, single stranded RNA, double stranded DNA, RNA-RNA hybrid, DNA-RNA hybrid, shortmer, antagomir, antisense, ribozyme, small interfering RNA (siRNA), asymmetrical interfering RNA (aiRNA), microRNA (miRNA), Dicer-substrate RNA (dsRNA), small hairpin RNA (shRNA), sgRNA/pegRNAs, transfer RNA (tRNA), messenger RNA (mRNA), or a combination thereof.

“Amino acid” and “amino acid residue” are used interchangeably.

“Peptide” refers to a compound comprising amino acid residues connected by peptide bonds. As used herein, a peptide compound has from 2 to 7 or more amino acid residues. In certain embodiments, a peptide compound has from 2 to about 50 amino acid residues.

“Polypeptide” refers to a compound comprising amino acid residues connected by peptide bonds. When the amino acids are alpha-amino acids, either the L-optical isomer or the D-optical isomer can be used. In some embodiments, a polypeptide has from about 50 amino acid residues to 2000 or more amino acid residues.

“Protein” refers to a molecule or complex comprising one or more polypeptides having secondary, tertiary and/or quaternary structure. The secondary, tertiary and/or quaternary structure of a protein typically is stabilized using non-covalent bonds, such as ionic bonds, hydrogen bonds, hydrophobic interactions, and/or van der Walls interactions. Additionally, or alternatively, a protein may include disulfide bonds, such as between the thiol groups of cysteine residues. In certain embodiments, a peptide, polypeptide, or protein comprises (e.g., consists essentially of) between 3 and 10, between 10 and 30, between 30 and 100, between 100 and 300, between 300 and 1,000, between 1,000 and 3,000, or between 3,000 and 10,000, inclusive, amino acids. In certain embodiments, the amino acids are natural amino acids. In certain embodiments, the amino acids are unnatural amino acids. In certain embodiments, the amino acids are L-amino acids. In certain embodiments, the amino acids are D-amino acids. In certain embodiments, the amino acids are canonical amino acids. In certain embodiments, the amino acids are non-canonical amino acids. In certain embodiments, the amino acids are selenocysteine or pyrrolysine. In certain embodiments, the amino acids are β-alanine, GABA, δ-aminolevulinic acid, α-aminoisobutyric acid, dehydroalanine, cystathionine, lanthionine, djenkolic acid, diaminopimelic acid, norvaline, norleucine, homonorleucine, O-methylhomoserine, O-ethylhomoserine, ethionine, ornithine, citrulline, γ-carboxyglutamate, hydroxyproline, hypusine, taurine, sarcosine, or glycine betaine. In certain embodiments, the amino acids are homo analogs of canonical amino acids. In certain embodiments, the amino acids are a combination of natural amino acids and unnatural amino acids (e.g., unnatural alpha-amino acids).

“Pharmaceutically acceptable excipient” refers to a substantially physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, as a carrier, flavoring, thickener, diluent, buffer, preservative, or surface active agent and/or to modify properties of a pharmaceutical composition. Examples of excipients include, but are not limited, to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

“Subject” refers to mammals and other animals, particularly humans. Thus disclosed methods are applicable to both human therapy and veterinary applications. In some embodiments, the subject is a human. In certain embodiments, the human is male or female, of any age group, e.g., pediatric subject (e.g., infant, child, or adolescent) or adult subject (e.g., young adult, middle-aged adult, or senior adult). In certain embodiments, the mammal is a primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal is a male or female at any stage of development. In some embodiments, the non-human animal is a transgenic animal or genetically engineered animal.

The term “tissue” refers to any biological tissue of a subject (including a group of cells, a body part, or an organ) or a part thereof, including blood and/or lymph vessels. In some embodiments, “tissue” is the object to which a compound, particle, and/or composition of the disclosure is delivered. In some embodiments, a tissue is an abnormal or unhealthy tissue, which may need to be treated. A tissue may also be a normal or healthy tissue that is under a higher than normal risk of becoming abnormal or unhealthy, which may need to be prevented.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound or composition described herein in a single dose. In certain embodiments, an effective amount is the combined amounts of a compound or composition described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adrenoleukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher's disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The cancer may be a solid tumor. The cancer may be a hematological malignancy. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, dermatitis (e.g., stasis dermatitis, allergic contact dermatitis, atopic dermatitis, irritant contact dermatitis, neurodermatitis perioral dermatitis, seborrheic dermatitis), hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, inflammatory rosacea. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrocephaly; megalocephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “metabolic disease” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

The term “psychiatric disorder” refers to a condition or disorder relating to the functioning of the brain and the cognitive processes or behavior. Psychiatric disorders may be further classified based on the type of neurological disturbance affecting the mental faculties. Psychiatric disorders are expressed primarily in abnormalities of thought, feeling, emotion, and/or behavior producing either distress or impairment of function (for example, impairment of mental function such with dementia or senility). The term “psychiatric disorder” is, accordingly, sometimes used interchangeably with the term “mental disorder” or the term “mental illness”. A psychiatric disorder is often characterized by a psychological or behavioral pattern that occurs in an individual and is thought to cause distress or disability that is not expected as part of normal development or culture. Definitions, assessments, and classifications of mental disorders can vary, but guideline criteria listed in the International Classification of Diseases and Related Health Problems (ICD, published by the World Health Organization, WHO), or the Diagnostic and Statistical Manual of Mental Disorders (DSM, published by the American Psychiatric Association, APA) and other manuals are widely accepted by mental health professionals. Individuals may be evaluated for various psychiatric disorders using criteria set forth in these and other publications accepted by medical practitioners in the field and the manifestation and severity of a psychiatric disorder may be determined in an individual using these publications.

### II. Introduction

Intravitreal administration of therapeutic agents is a less invasive and ambulatory procedure that provides an attractive alternative to the established subretinal route without its complications. Associated benefits of this route of administration include increased therapeutic volume for retina-wide delivery leading to increased therapeutic efficacy, minimized risk of submacular hemorrhage or retinal detachment stemming from surgical procedure, and lower costs coupled with shorter recovery times for patients and hospitals. Although Adeno-Associated Viruses (AAVs) are now also being extensively explored for intravitreal administration, their possible immunogenicity, inability to encapsulate large genes, as well as manufacturing difficulties at large scales and physiological barriers of the eye pose a challenge to their efficacy and successful targeting. Non-viral carriers like lipid nanoparticles (LNPs) are biocompatible and can package large cargo size while eliciting efficient delivery within cells, yet they still lack the ever-crucial cell-specific active targeting capabilities. As with AAVs, LNPs need to penetrate the viscous entanglement of collagen fibrils and carbohydrates that make up the vitreous humor as well as vitreoretinal inner limiting membrane, anatomically inherent to the eye separating vitreous cavity from retina thereby limiting the ability of these LNPs to reach PR and RPE. The varied limitations of the different delivery systems available to date highlight the importance of finding alternative successful approaches that can deliver efficiently while minimizing adverse effects.

In terms of targeted delivery of pharmaceutically-relevant cargo, peptides are ligands that can be endogenous and exogenously derived, are modifiable, easy to synthesize and purify at large scales and best of all, have proven therapeutic efficacy, cell-specificity, and exhibit penetrative properties as seen in nanotechnology and cancer fields for some time now. Moreover, the simplicity of peptide sequences allows for rational evolution of side chains for improved binding and targeting affinities making them prime candidates for intraocular targeted delivery. If one combines the favorable properties exhibited by peptides with the shortcomings of current IRD therapies, these ligands emerge as an appealing strategy for bioconjugation with varied delivery fields such as viral and non-viral gene and drug delivery, nucleic acid-conjugated delivery, and even can prove useful as a diagnostic agent if employed for tissue or cell-specific imaging all in the name of advancing the field of targeted ophthalmic therapy.

With these goals in mind, a combinatorial M13-bacteriophage heptameric peptide phage display library was used for mining retina-traversing peptide ligands for specific targeting to PRs and RPE following intravitreal delivery. Disclosed herein are peptide candidates resulting from mouse biopanning rounds, that were further evaluated by their binding affinities to specific PR and RPE model cell lines. In vivo targeting of PRs and RPE layers confirmed the results and elicited the potential for use in combination with established gene and drug delivery platforms such as lipid nanoparticles (LNPs). Physicochemical characteristics as well as structural motifs of the disclosed targeting ligands were elucidated to complement differential binding information in order to further showcase key motifs important for penetration of vitreous and targeting following intravitreal delivery. These peptide candidates not only have the potential of providing improved specificity to current viral and non-viral vectors for therapeutic cargo delivery to the retina for gene replacement, but could also be conjugated to existing nucleic acid modalities directly for gene knockdown as is the case with small interfering RNAs (siRNAs) to address different genetic ailments. Additionally, these peptide moieties have the potential in aiding the ophthalmic imaging field for therapy progression analysis or disease diagnosis if conjugated to a therapeutically useful fluorophore.

### III. Peptides and Conjugates

Disclosed herein are peptides that are useful as retina targeting ligands. The peptides may be useful as targeting ligands for delivering therapeutic and/or imaging agents to the retina. Possible applications include, but are not limited to, gene and/or drug delivery platforms such as lipid nanoparticles, imaging agents (for example, fluorophores, such as TAMRA fluorophore, or radiotracers), gene therapy modulation (for example, silencing genes using siRNA or miRNA), overexpression (for example, using LNPs decorated with peptides or even viruses and their capsids decorated with these same peptides), gene editing (for example, using LNPs and/or viral carriers that need targeting to PRs in retina).

As described herein, the amino acid residues present in the disclosed peptides may be defined as follows:
Hydrophobic: Ala, Val, Gly, Ile, Leu, Phe, Pro, Trp, Tyr, Met, Cys.
Hydrophilic: Arg, Asn, Asp, Glu, Gln, Lys, Ser, Thr, His.
Non Polar: Ala, Gly, Ile, Leu, Phe, Val, Pro.
Amphipathic: Trp, Tyr, Met.
Polar: Ser, Thr, Asn, Gln, Arg, Lys, Asp, His, Cys, Glu.
Basic: Arg, Lys, His.
Acidic: Asp, Glu.

Additionally, the amino acid residues may be described in terms of more than one of the above definitions. For example, an amino acid residue may be described as being “polar/hydrophilic.” This means that the amino acid residue is selected from the set of amino acid residues that belong to both the polar and hydrophilic groups above. That is, a polar/hydrophilic residue is selected from Ser, Thr, Asn, Gln, Arg, Lys, Asp, His, or Glu, but not Cys, because Cys is not present in both of the polar and hydrophilic groups. And a “polar/hydrophilic/basic” amino acid residue is selected from Lys or His because they are the only residues that are present in all of the polar, hydrophilic and basic groups above.

In some embodiments, the present disclosure provides peptides according to Formula I or V:

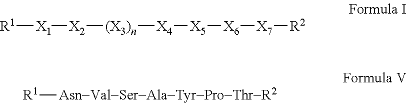

wherein:
n is 0, 1, or 2;
X₁ is a polar and hydrophilic amino acid, or amphipathic and hydrophobic amino acid, or non-polar and hydrophobic amino acid;
X₂ is a non-polar and hydrophobic amino acid or polar and hydrophilic amino acid;
If present, each X₃ independently is a non-polar and hydrophobic amino acid or polar and hydrophilic amino acid;
X₄ is a non-polar and hydrophobic amino acid or polar and hydrophilic amino acid;
X₅ is a polar and hydrophilic amino acid or non-polar and hydrophobic amino acid;
X₆ is a non-polar and hydrophobic amino acid, or amphipathic and hydrophobic amino acid, or polar and hydrophilic amino acid;
X₇ is a non-polar and hydrophobic amino acid or polar and hydrophilic and basic amino acid; and
R¹ is H, an amino acid, a linker moiety, or a nitrogen protecting group;
R² is OH, an amino acid, a linker moiety, or O(an oxygen protecting group);
   and wherein the amino acids are defined as follows:
each hydrophobic amino acid is independently: Ala, Val, Gly, Ile, Leu, Phe, Pro, Trp, Tyr, Met, or Cys;
each hydrophilic amino acid is independently: Arg, Asn, Asp, Glu, Gln, Lys, Ser, Thr, or His;
each non-polar amino acid is independently: Ala, Gly, Ile, Leu, Phe, Val, or Pro;
each amphipathic amino acid is independently: Trp, Tyr, or Met;
each polar amino acid is independently: Ser, Thr, Asn, Gln, Arg, Lys, Asp, His, Cys, or Glu;
each basic amino acid is independently: Arg, Lys, or His; and
each acidic amino acid is independently: Asp or Glu.

In some embodiments, a disclosed peptide may have a structure according to Formula I:

R¹—X₁—X₂—(X₃)ₙ—X₄—X₅—X₆—X₇—R² Formula I.

In some embodiments, n is 0, 1 or 2;
X₁ is polar/hydrophilic or amphipathic/hydrophobic or non-polar/hydrophobic;
X₂ is non-polar/hydrophobic or polar/hydrophilic, and may be optionally substituted with Cys;
if present, each X₃ independently is non-polar/hydrophobic or polar/hydrophilic;
X₄ is non-polar/hydrophobic or polar/hydrophilic;
X₅ is polar/hydrophilic or non-polar/hydrophobic;
X₆ is non-polar/hydrophobic or amphipathic/hydrophobic or polar/hydrophilic;
X₇ is non-polar/hydrophobic or polar/hydrophilic/basic, and may be optionally substituted with Cys;
R¹ is H, an amino acid residue, or a linker moiety; and
R² is OH, an amino acid residue, or a linker moiety.

In some embodiments, X₁ is Ala, Asp, Glu, Phe, Gly, His, Ile, Lys, Leu, Met, Asn, Pro, Gln, Arg, Ser, Thr, Val, Trp, or Tyr;
each of X₂, X₃, X₄, and X₅ is independently Ala, Asp, Glu, Phe, Gly, His, Ile, Lys, Leu, Asn, Pro, Gln, Arg, Ser, Thr, or Val;
X₆ is Ala, Asp, Glu, Phe, Gly, His, Ile, Lys, Leu, Met, Asn, Pro, Gln, Arg, Ser, Thr, Val, Trp, or Tyr; and
X₇ is Ala, Phe, Gly, His, Ile, Lys, Leu, Pro, Arg, or Val.

In some embodiments, X₁ is polar/hydrophilic/basic or amphipathic/hydrophobic or non-polar/hydrophobic.

In some embodiments, X₅ is polar/hydrophilic/basic or non-polar/hydrophobic.

Additionally or alternatively, any of X₁ through X₇ may be an non-natural amino acid residue derived from a non-natural amino acid, such as a D-amino acid and/or a synthetic amino acid having a different formula from a natural amino acid. Additionally or alternatively, the peptide may comprise one or more pseudo peptide bonds between any two adjacent amino acid residues. Additionally information concerning such modifications can be found, for example, in Gentilucci et al., Current Pharmaceutical Design, 2010, 16, 3185-3203, which is incorporated herein by reference.

Additionally, in some embodiments, one or more of the following conditions may apply:
a) the peptide contains one or more polar amino acid residues;
b) the peptide contains at least one hydrophobic amino acid residue;
c) the peptide contains 1-3 hydrophobic amino acid residues;
d) the peptide contains 1-5 non-polar amino acid residues;
e) the peptide does not contain an acid amino acid residue.

In some embodiments:
X₁ is non-polar/hydrophobic or Ser or Met;
X₂ is non-polar/hydrophobic or Asn or Thr;
If present, each X₃ independently is non-polar/hydrophobic or Ser;
X₄ is non-polar/hydrophobic or Ser;
X₅ is non-polar/hydrophobic or polar/hydrophobic/basic, such as non-polar/hydrophobic or Arg or His;
X₆ is non-polar/hydrophobic or polar/hydrophobic or Tyr;
X₇ is non-polar/hydrophobic or Arg; or
a combination thereof.

In one embodiment, X₁ is polar/hydrophilic, X₂ is non-polar/hydrophobic, X₃ is non-polar/hydrophobic, X₄ is non-polar/hydrophobic, X₅ is polar/hydrophilic/basic, X₆ is non-polar/hydrophobic, and X₇ is non-polar/hydrophobic.

In one embodiment, X₁ is polar/hydrophilic, X₂ is polar/hydrophilic, X₃ is non-polar/hydrophobic, X₄ is non-polar/hydrophobic, X₅ is non-polar/hydrophobic, X₆ is non-polar/hydrophobic, and X₇ is non-polar/hydrophobic.

In one embodiment, X₁ is amphipatic/hydrophobic, X₂ is non-polar/hydrophobic, X₃ is non-polar/hydrophobic, X₄ is non-polar/hydrophobic, X₅ is non-polar/hydrophobic, X₆ is amphipatic/hydrophobic, and X₇ is polar/hydrophilic/basic.

In one embodiment, X₁ is non-polar/hydrophobic, X₂ is polar/hydrophilic, X₃ is non-polar/hydrophobic, X₄ is non-polar/hydrophobic, X₅ is polar/hydrophilic/basic, X₆ is polar/hydrophilic, and X₇ is non-polar/hydrophobic.

In one embodiment, X₁ is polar/hydrophilic/basic, X₂ is non-polar/hydrophobic, X₃ is polar/hydrophilic, X₄ is polar/hydrophilic, X₅ is non-polar/hydrophobic, X₆ is polar/hydrophilic, and X₇ is non-polar/hydrophobic.

In certain embodiments:
X₁ is Ser, Met, Ala or His;
X₂ is Pro, Asn, Thr or Leu;
If present, each X₃ independently is Ala, Leu, Val, Gly, or Ser;
X₄ is Leu, Ala, Pro, or Ser;
X₅ is His, Ala, Val, Arg, or Leu;
X₆ is Phe, Tyr, Ser, or Thr;
X₇ is Leu, Pro, Arg, Val, or Pro; or
a combination thereof.

In certain embodiments:
X₁ is Ser, Met, Ala or His;
X₂ is Pro, Asn, Thr or Leu;
If present, each X₃ independently is Ala, Leu, Val, Gly, or Ser;
X₄ is Leu, Ala, Pro, or Ser;
X₅ is His, Ala, Val, Arg, or Leu;
X₆ is Phe, Tyr, Ser, or Thr; or
X₇ is Leu, Pro, Arg, Val, or Pro; or
a combination thereof.

In certain embodiments, X₁ is Ser, Met, Ala, or His.

In certain embodiments, X₂ is Pro, Asn, Thr, or Leu. In certain embodiments, X₂ is Pro, Asn, Ala, or Leu. In certain embodiments, X₂ is Pro, Asn, Ala, Thr, or Leu.

In certain embodiments, if present, each X₃ independently is Ala, Leu, Val, Gly, or Ser. In certain embodiments, if present, each X₃ independently is Ala, Leu, Val, Phe, or Ser. In certain embodiments, if present, each X₃ independently is Ala, Leu, Val, Gly, Phe, or Ser.

In certain embodiments, X₄ is Leu, Ala, Pro, or Ser. In certain embodiments, X₄ is Leu, Ala, His, or Ser. In certain embodiments, X₄ is Leu, Ala, Pro, His, or Ser.

In certain embodiments, X₅ is His, Ala, Val, Arg, or Leu.

In certain embodiments, X₆ is Phe, Tyr, Ser, or Thr. In certain embodiments, X₆ is Phe, Tyr, Met, or Thr. In certain embodiments, X₆ is Phe, Tyr, Ser, Met, or Thr.

In certain embodiments, X₇ is Leu, Pro, Arg, Val, or Pro. In certain embodiments, X₇ is Leu, Pro, Arg, or Pro.

In certain embodiments, one or more (e.g., 2 or 3) of X₁ to X₇ is a basic amino acid. In certain embodiments, one or more (e.g., 2 or 3) of X₁ to X₇ is His. In certain embodiments, only one of X₁ to X₇ is a basic amino acid. In certain embodiments, only one of X₁ to X₇ is His.

In any of the above embodiments, n may be 1.

In some embodiments, X₁ to X₇ are as shown in the table below:

| | | | Each X₃ | | | | |
|---|---|---|---|---|---|---|---|
| | | | independently | | | | |
| No. | X₁ | X₂ | (e.g., n is 1) | X₄ | X₅ | X₆ | X₇ |
| 42-1 | Ala, Asp, | Pro | Ala | Leu | His | Phe | Leu |
| (SEQ | Glu, Phe, | | | | | | |
| ID | Gly, His, | | | | | | |
| NO: | Ile, Lys, | | | | | | |
| 57) | Leu, Met, | | | | | | |
| | Asn, Pro, | | | | | | |
| | Gln, Arg, | | | | | | |
| | Ser, Thr, | | | | | | |
| | Val, Trp, | | | | | | |
| | or Tyr | | | | | | |
| 42-2 | Ser | Ala, Asp, | Ala | Leu | His | Phe | Leu |
| (SEQ | | Glu, Phe, | | | | | |
| ID | | Gly, His, | | | | | |
| NO: | | Ile, Lys, | | | | | |
| 58) | | Leu, Asn, | | | | | |
| | | Pro, Gln, | | | | | |
| | | Arg, Ser, | | | | | |
| | | Thr, or | | | | | |
| | | Val | | | | | |
| 42-3 | Ser | Pro | Ala, Asp, Glu, | Leu | His | Phe | Leu |
| (SEQ | | | Phe, Gly, His, | | | | |
| ID | | | Ile, Lys, Leu, | | | | |
| NO: | | | Asn, Pro, Gln, | | | | |
| 59) | | | Arg, Ser, Thr, | | | | |
| | | | or Val | | | | |
| 42-4 | Ser | Pro | Ala | Ala, Asp, | His | Phe | Leu |
| (SEQ | | | | Glu, Phe, | | | |
| ID | | | | Gly, His, | | | |
| NO: | | | | Ile, Lys, | | | |
| 60) | | | | Leu, Asn, | | | |
| | | | | Pro, Gln, | | | |
| | | | | Arg, Ser, | | | |
| | | | | Thr, or | | | |
| | | | | Val | | | |
| 42-5 | Ser | Pro | Ala | Leu | Ala, Asp, | Phe | Leu |
| (SEQ | | | | | Glu, Phe, | | |
| ID | | | | | Gly, His, | | |
| NO: | | | | | Ile, Lys, | | |
| 61) | | | | | Leu, Asn, | | |
| | | | | | Pro, Gln, | | |
| | | | | | Arg, Ser, | | |
| | | | | | Thr, or | | |
| | | | | | Val | | |
| 42-6 | Ser | Pro | Ala | Leu | His | Ala, Asp, | Leu |
| (SEQ | | | | | | Glu, Phe, | |
| ID | | | | | | Gly, His, | |
| NO: | | | | | | Ile, Lys, | |
| 62) | | | | | | Leu, Met, | |
| | | | | | | Asn, Pro, | |
| | | | | | | Gln, Arg, | |
| | | | | | | Ser, Thr, | |
| | | | | | | Val, Trp, | |
| | | | | | | or Tyr | |
| 42-7 | Ser | Pro | Ala | Leu | His | Phe | Ala, Phe, |
| (SEQ | | | | | | | Gly, His, |
| ID | | | | | | | Ile, Lys, |
| NO: | | | | | | | Leu, Pro, |
| 63) | | | | | | | Arg, or |
| | | | | | | | Val. |

In some embodiments, X₁ to X₇ are as shown in the table below:

| | | | Each X₃ | | | | |
|---|---|---|---|---|---|---|---|
| | | | independently | | | | |
| No. | X₁ | X₂ | (e.g., n is 1) | X₄ | X₅ | X₆ | X₇ |
| 43-1 | Ala, Asp, | Asn | Leu | Ala | Ala | Phe | Pro |
| (SEQ | Glu, Phe, | | | | | | |
| ID | Gly, His, | | | | | | |
| NO: | Ile, Lys, | | | | | | |
| 64) | Leu, Met, | | | | | | |
| | Asn, Pro, | | | | | | |
| | Gln, Arg, | | | | | | |
| | Ser, Thr, | | | | | | |
| | Val, Trp, | | | | | | |
| | or Tyr | | | | | | |
| 43-2 | Ser | Ala, Asp, | Leu | Ala | Ala | Phe | Pro |
| (SEQ | | Glu, Phe, | | | | | |
| ID | | Gly, His, | | | | | |
| NO: | | Ile, Lys, | | | | | |
| 65) | | Leu, Asn, | | | | | |
| | | Pro, Gln, | | | | | |
| | | Arg, Ser, | | | | | |
| | | Thr, or | | | | | |
| | | Val | | | | | |
| 43-3 | Ser | Asn | Ala, Asp, Glu, | Ala | Ala | Phe | Pro |
| (SEQ | | | Phe, Gly, His, | | | | |
| ID | | | Ile, Lys, Leu, | | | | |
| NO: | | | Asn, Pro, Gln, | | | | |
| 66) | | | Arg, Ser, Thr, | | | | |
| | | | or Val | | | | |
| 43-4 | Ser | Asn | Leu | Ala, Asp, | Ala | Phe | Pro |
| (SEQ | | | | Glu, Phe, | | | |
| ID | | | | Gly, His, | | | |
| NO: | | | | Ile, Lys, | | | |
| 67) | | | | Leu, Asn, | | | |
| | | | | Pro, Gln, | | | |
| | | | | Arg, Ser, | | | |
| | | | | Thr, or | | | |
| | | | | Val | | | |
| 43-5 | Ser | Asn | Leu | Ala | Ala, Asp, | Phe | Pro |
| (SEQ | | | | | Glu, Phe, | | |
| ID | | | | | Gly, His, | | |
| NO: | | | | | Ile, Lys, | | |
| 68) | | | | | Leu, Asn, | | |
| | | | | | Pro, Gln, | | |
| | | | | | Arg, Ser, | | |
| | | | | | Thr, or | | |
| | | | | | Val | | |
| 43-6 | Ser | Asn | Leu | Ala | Ala | Ala, Asp, | Pro |
| (SEQ | | | | | | Glu, Phe, | |
| ID | | | | | | Gly, His, | |
| NO: | | | | | | Ile, Lys, | |
| 69) | | | | | | Leu, Met, | |
| | | | | | | Asn, Pro, | |
| | | | | | | Gln, Arg, | |
| | | | | | | Ser, Thr, | |
| | | | | | | Val, Trp, | |
| | | | | | | or Tyr | |
| 43-7 | Ser | Asn | Leu | Ala | Ala | Phe | Ala, Phe, |
| (SEQ | | | | | | | Gly, His, |
| ID | | | | | | | Ile, Lys, |
| NO: | | | | | | | Leu, Pro, |
| 70) | | | | | | | Arg, or |
| | | | | | | | Val. |

In some embodiments, X₁ to X₇ are as shown in the table below:

| | | | Each X₃ | | | | |
|---|---|---|---|---|---|---|---|
| | | | independently | | | | |
| No. | X₁ | X₂ | (e.g., n is 1) | X₄ | X₅ | X₆ | X₇ |
| 50-1 | Ala, Asp, | Pro | Val | Ala | Val | Tyr | Arg |
| (SEQ | Glu, Phe, | | | | | | |
| ID | Gly, His, | | | | | | |
| NO: | Ile, Lys, | | | | | | |
| 71) | Leu, Met, | | | | | | |
| | Asn, Pro, | | | | | | |
| | Gln, Arg, | | | | | | |
| | Ser, Thr, | | | | | | |
| | Val, Trp, | | | | | | |
| | or Tyr | | | | | | |
| 50-2 | Met | Ala, Asp, | Val | Ala | Val | Tyr | Arg |
| (SEQ | | Glu, Phe, | | | | | |
| ID | | Gly, His, | | | | | |
| NO: | | Ile, Lys, | | | | | |
| 72) | | Leu, Asn, | | | | | |
| | | Pro, Gln, | | | | | |
| | | Arg, Ser, | | | | | |
| | | Thr, or | | | | | |
| | | Val | | | | | |
| 50-3 | Met | Pro | Ala, Asp, Glu, | Ala | Val | Tyr | Arg |
| (SEQ | | | Phe, Gly, His, | | | | |
| ID | | | Ile, Lys, Leu, | | | | |
| NO: | | | Asn, Pro, Gln, | | | | |
| 73) | | | Arg, Ser, Thr, | | | | |
| | | | or Val | | | | |
| 50-4 | Met | Pro | Val | Ala, Asp, | Val | Tyr | Arg |
| (SEQ | | | | Glu, Phe, | | | |
| ID | | | | Gly, His, | | | |
| NO: | | | | Ile, Lys, | | | |
| 74) | | | | Leu, Asn, | | | |
| | | | | Pro, Gln, | | | |
| | | | | Arg, Ser, | | | |
| | | | | Thr, or | | | |
| | | | | Val | | | |
| 50-5 | Met | Pro | Val | Ala | Ala, Asp, | Tyr | Arg |
| (SEQ | | | | | Glu, Phe, | | |
| ID | | | | | Gly, His, | | |
| NO: | | | | | Ile, Lys, | | |
| 75) | | | | | Leu, Asn, | | |
| | | | | | Pro, Gln, | | |
| | | | | | Arg, Ser, | | |
| | | | | | Thr, or | | |
| | | | | | Val | | |
| 50-6 | Met | Pro | Val | Ala | Val | Ala, Asp, | Arg |
| (SEQ | | | | | | Glu, Phe, | |
| ID | | | | | | Gly, His, | |
| NO: | | | | | | Ile, Lys, | |
| 76) | | | | | | Leu, Met, | |
| | | | | | | Asn, Pro, | |
| | | | | | | Gln, Arg, | |
| | | | | | | Ser, Thr, | |
| | | | | | | Val, Trp, | |
| | | | | | | or Tyr | |
| 50-7 | Met | Pro | Val | Ala | Val | Tyr | Ala, Phe, |
| (SEQ | | | | | | | Gly, His, |
| ID | | | | | | | Ile, Lys, |
| NO: | | | | | | | Leu, Pro, |
| 77) | | | | | | | Arg, or |
| | | | | | | | Val. |

In some embodiments, X₁ to X₇ are as shown in the table below:

| | | | Each X₃ | | | | |
|---|---|---|---|---|---|---|---|
| | | | independently | | | | |
| No. | X₁ | X₂ | (e.g., n is 1) | X₄ | X₅ | X₆ | X₇ |
| 52-1 | Ala, Asp, | Ala | Phe | His | Arg | Met | Pro |
| (SEQ | Glu, Phe, | | | | | | |
| ID | Gly, His, | | | | | | |
| NO: | Ile, Lys, | | | | | | |
| 78) | Leu, Met, | | | | | | |
| | Asn, Pro, | | | | | | |
| | Gln, Arg, | | | | | | |
| | Ser, Thr, | | | | | | |
| | Val, Trp, | | | | | | |
| | or Tyr | | | | | | |
| 52-2 | Leu | Ala, Asp, | Phe | His | Arg | Met | Pro |
| (SEQ | | Glu, Phe, | | | | | |
| ID | | Gly, His, | | | | | |
| NO: | | Ile, Lys, | | | | | |
| 79) | | Leu, Asn, | | | | | |
| | | Pro, Gln, | | | | | |
| | | Arg, Ser, | | | | | |
| | | Thr, or | | | | | |
| | | Val | | | | | |
| 52-3 | Leu | Ala | Ala, Asp, Glu, | His | Arg | Met | Pro |
| (SEQ | | | Phe, Gly, His, | | | | |
| ID | | | Ile, Lys, Leu, | | | | |
| NO: | | | Asn, Pro, Gln, | | | | |
| 80) | | | Arg, Ser, Thr, | | | | |
| | | | or Val | | | | |
| 52-4 | Leu | Ala | Phe | Ala, Asp, | Arg | Met | Pro |
| (SEQ | | | | Glu, Phe, | | | |
| ID | | | | Gly, His, | | | |
| NO: | | | | Ile, Lys, | | | |
| 81) | | | | Leu, Asn, | | | |
| | | | | Pro, Gln, | | | |
| | | | | Arg, Ser, | | | |
| | | | | Thr, or | | | |
| | | | | Val | | | |
| 52-5 | Leu | Ala | Phe | His | Ala, Asp, | Met | Pro |
| (SEQ | | | | | Glu, Phe, | | |
| ID | | | | | Gly, His, | | |
| NO: | | | | | Ile, Lys, | | |
| 82) | | | | | Leu, Asn, | | |
| | | | | | Pro, Gln, | | |
| | | | | | Arg, Ser, | | |
| | | | | | Thr, or | | |
| | | | | | Val | | |
| 52-6 | Leu | Ala | Phe | His | Arg | Ala, Asp, | Pro |
| (SEQ | | | | | | Glu, Phe, | |
| ID | | | | | | Gly, His, | |
| NO: | | | | | | Ile, Lys, | |
| 83) | | | | | | Leu, Met, | |
| | | | | | | Asn, Pro, | |
| | | | | | | Gln, Arg, | |
| | | | | | | Ser, Thr, | |
| | | | | | | Val, Trp, | |
| | | | | | | or Tyr | |
| 52-7 | Leu | Ala | Phe | His | Arg | Met | Ala, Phe, |
| (SEQ | | | | | | | Gly, His, |
| ID | | | | | | | Ile, Lys, |
| NO: | | | | | | | Leu, Pro, |
| 84) | | | | | | | Arg, or |
| | | | | | | | Val. |

In some embodiments, X₁ to X₇ are as shown in the table below:

| | | | Each X₃ | | | | |
|---|---|---|---|---|---|---|---|
| | | | independently | | | | |
| No. | X₁ | X₂ | (e.g., n is 1) | X₄ | X₅ | X₆ | X₇ |
| 54-1 | Ala, Asp, | Leu | Ser | Ser | Leu | Thr | Pro |
| (SEQ | Glu, Phe, | | | | | | |
| ID | Gly, His, | | | | | | |
| NO: | Ile, Lys, | | | | | | |
| 85) | Leu, Met, | | | | | | |
| | Asn, Pro, | | | | | | |
| | Gln, Arg, | | | | | | |
| | Ser, Thr, | | | | | | |
| | Val, Trp, | | | | | | |
| | or Tyr | | | | | | |
| 54-2 | His | Ala, Asp, | Ser | Ser | Leu | Thr | Pro |
| (SEQ | | Glu, Phe, | | | | | |
| ID | | Gly, His, | | | | | |
| NO: | | Ile, Lys, | | | | | |
| 86) | | Leu, Asn, | | | | | |
| | | Pro, Gln, | | | | | |
| | | Arg, Ser, | | | | | |
| | | Thr, or | | | | | |
| | | Val | | | | | |
| 54-3 | His | Leu | Ala, Asp, Glu, | Ser | Leu | Thr | Pro |
| (SEQ | | | Phe, Gly, His, | | | | |
| ID | | | Ile, Lys, Leu, | | | | |
| NO: | | | Asn, Pro, Gln, | | | | |
| 87) | | | Arg, Ser, Thr, | | | | |
| | | | or Val | | | | |
| 54-4 | His | Leu | Ser | Ala, Asp, | Leu | Thr | Pro |
| (SEQ | | | | Glu, Phe, | | | |
| ID | | | | Gly, His, | | | |
| NO: | | | | Ile, Lys, | | | |
| 88) | | | | Leu, Asn, | | | |
| | | | | Pro, Gln, | | | |
| | | | | Arg, Ser, | | | |
| | | | | Thr, or | | | |
| | | | | Val | | | |
| 54-5 | His | Leu | Ser | Ser | Ala, Asp, | Thr | Pro |
| (SEQ | | | | | Glu, Phe, | | |
| ID | | | | | Gly, His, | | |
| NO: | | | | | Ile, Lys, | | |
| 89) | | | | | Leu, Asn, | | |
| | | | | | Pro, Gln, | | |
| | | | | | Arg, Ser, | | |
| | | | | | Thr, or | | |
| | | | | | Val | | |
| 54-6 | His | Leu | Ser | Ser | Leu | Ala, Asp, | Pro |
| (SEQ | | | | | | Glu, Phe, | |
| ID | | | | | | Gly, His, | |
| NO: | | | | | | Ile, Lys, | |
| 90) | | | | | | Leu, Met, | |
| | | | | | | Asn, Pro, | |
| | | | | | | Gln, Arg, | |
| | | | | | | Ser, Thr, | |
| | | | | | | Val, Trp, | |
| | | | | | | or Tyr | |
| 54-7 | His | Leu | Ser | Ser | Leu | Thr | Ala, Phe, |
| (SEQ | | | | | | | Gly, His, |
| ID | | | | | | | Ile, Lys, |
| NO: | | | | | | | Leu, Pro, |
| 91) | | | | | | | Arg, or |
| | | | | | | | Val. |

In some embodiments, X₁ to X₇ are as shown in the table below:

| | | | Each X₃ | | | | |
|---|---|---|---|---|---|---|---|
| | | | independently | | | | |
| No. | X₁ | X₂ | (e.g., n is 1) | X₄ | X₅ | X₆ | X₇ |
| 57-1 | Ala, Asp, | Thr | Gly | Pro | Arg | Ser | Val |
| (SEQ | Glu, Phe, | | | | | | |
| ID | Gly, His, | | | | | | |
| NO: | Ile, Lys, | | | | | | |
| 92) | Leu, Met, | | | | | | |
| | Asn, Pro, | | | | | | |
| | Gln, Arg, | | | | | | |
| | Ser, Thr, | | | | | | |
| | Val, Trp, | | | | | | |
| | or Tyr | | | | | | |
| 57-2 | Ala | Ala, Asp, | Gly | Pro | Arg | Ser | Val |
| (SEQ | | Glu, Phe, | | | | | |
| ID | | Gly, His, | | | | | |
| NO: | | Ile, Lys, | | | | | |
| 93) | | Leu, Asn, | | | | | |
| | | Pro, Gln, | | | | | |
| | | Arg, Ser, | | | | | |
| | | Thr, or | | | | | |
| | | Val | | | | | |
| 57-3 | Ala | Thr | Ala, Asp, Glu, | Pro | Arg | Ser | Val |
| (SEQ | | | Phe, Gly, His, | | | | |
| ID | | | Ile, Lys, Leu, | | | | |
| NO: | | | Asn, Pro, Gln, | | | | |
| 94) | | | Arg, Ser, Thr, | | | | |
| | | | or Val | | | | |
| 57-4 | Ala | Thr | Gly | Ala, Asp, | Arg | Ser | Val |
| (SEQ | | | | Glu, Phe, | | | |
| ID | | | | Gly, His, | | | |
| NO: | | | | Ile, Lys, | | | |
| 95) | | | | Leu, Asn, | | | |
| | | | | Pro, Gln, | | | |
| | | | | Arg, Ser, | | | |
| | | | | Thr, or | | | |
| | | | | Val | | | |
| 57-5 | Ala | Thr | Gly | Pro | Ala, Asp, | Ser | Val |
| (SEQ | | | | | Glu, Phe, | | |
| ID | | | | | Gly, His, | | |
| NO: | | | | | Ile, Lys, | | |
| 96) | | | | | Leu, Asn, | | |
| | | | | | Pro, Gln, | | |
| | | | | | Arg, Ser, | | |
| | | | | | Thr, or | | |
| | | | | | Val | | |
| 57-6 | Ala | Thr | Gly | Pro | Arg | Ala, Asp, | Val |
| (SEQ | | | | | | Glu, Phe, | |
| ID | | | | | | Gly, His, | |
| NO: | | | | | | Ile, Lys, | |
| 97) | | | | | | Leu, Met, | |
| | | | | | | Asn, Pro, | |
| | | | | | | Gln, Arg, | |
| | | | | | | Ser, Thr, | |
| | | | | | | Val, Trp, | |
| | | | | | | or Tyr | |
| 57-7 | Ala | Thr | Gly | Pro | Arg | Ser | Ala, Phe, |
| (SEQ | | | | | | | Gly, His, |
| ID | | | | | | | Ile, Lys, |
| NO: | | | | | | | Leu, Pro, |
| 98) | | | | | | | Arg, or |
| | | | | | | | Val. |

In some embodiments, X₁ to X₇ are as shown in the table below:

| | | | Each X₃ | | | | |
|---|---|---|---|---|---|---|---|
| No. | X₁ | X₂ | (e.g., n is 1) | X₄ | X₅ | X₆ | X₇ |
| 42-8 | Ser | Pro | Ala | Leu | His | Phe | Leu |
| (SEQ | | | | | | | |
| ID | | | | | | | |
| NO: | | | | | | | |
| 23) | | | | | | | |
| 43-8 | Ser | Asn | Leu | Ala | Ala | Phe | Pro |
| (SEQ | | | | | | | |
| ID | | | | | | | |
| NO: | | | | | | | |
| 24) | | | | | | | |
| 50-8 | Met | Pro | Val | Ala | Val | Tyr | Arg |
| (SEQ | | | | | | | |
| ID | | | | | | | |
| NO: | | | | | | | |
| 26) | | | | | | | |
| 52-8 | Leu | Ala | Phe | His | Arg | Met | Pro |
| (SEQ | | | | | | | |
| ID | | | | | | | |
| NO: | | | | | | | |
| 27) | | | | | | | |
| 54-8 | His | Leu | Ser | Ser | Leu | Thr | Pro |
| (SEQ | | | | | | | |
| ID | | | | | | | |
| NO: | | | | | | | |
| 28) | | | | | | | |
| 57-8 | Ala | Thr | Gly | Pro | Arg | Ser | Val. |
| (SEQ | | | | | | | |
| ID | | | | | | | |
| NO: | | | | | | | |
| 29) | | | | | | | |

In particular embodiments, the peptide is selected from:

Peptide 42:
(SEQ ID NO. 23)
Ser-Pro-Ala-Leu-His-Phe-Leu
Peptide 43:
(SEQ ID No. 24)
Ser-Asn-Leu-Ala-Ala-Phe-Pro
Peptide 50:
(SEQ ID No. 26)
Met-Pro-Val-Ala-Val-Tyr-Arg
Peptide 57:
(SEQ ID No. 29)
Ala-Thr-Gly-Pro-Arg-Ser-Val
Peptide 54:
(SEQ ID No. 28)
His-Leu-Ser-Ser-Leu-Thr-Pro.

In some embodiments, the peptide is Peptide 42: Ser-Pro-Ala-Leu-His-Phe-Leu (SEQ ID No. 23). In some embodiments, the peptide is Peptide 52: Leu-Ala-Phe-His-Arg-Met-Pro (SEQ ID No. 27).

In certain embodiments, the peptide is of Formula V.

In some embodiments, R¹ is H. In some embodiments, R¹ is a nitrogen protecting group. In some embodiments, R¹ is a linker moiety. In some embodiments, R² is OH. In some embodiments, R² is O(oxygen protecting group). In some embodiments, R² is a linker moiety. In some embodiments, R¹ is H and R² is OH. In certain embodiments, R¹ is H or a nitrogen protecting group, and R² is OH or O(an oxygen protecting group). In other embodiments, R¹ is H and R² is a linker moiety. In certain embodiments, R¹ is H or a nitrogen protecting group, and R² is the linker moiety. In certain embodiments, R¹ is the linker moiety and R² is OH. In certain embodiments, R¹ is the linker moiety and R² is OH or O(an oxygen protecting group). In any embodiments, the linker moiety is any moiety suitable to attach the peptide to a desired agent such as a therapeutic (for example, but not limited to, a nucleic acid, antibody, small molecule, imaging agent and/or a nanoparticle that contains therapeutic modality). In some embodiments, the linker moiety is a short peptide sequence. Typically, such a linker sequence comprises small, non-polar (for example, Gly) or polar (for example, Ser, Thr) amino acids. For example, the linker peptide sequence may comprise 2 to 7 amino acid residues, or 4 to 6 residues. In other embodiments, the linker moiety may comprise a repeated sequence, for example, in (GGGS)ₙ or others such as (GGGGS)ₙ where n can be 2, 3, 4. Other alternatives could be just (Gly)₈ or (Gly)₆. In some embodiments, the linker moiety is a peptide sequence of 5 amino acid residues. In any embodiments, the linker moiety may comprise a carboxy group and or one or more Cys residues, such as a terminal Cys residue.

In certain embodiments, the linker moiety is substituted or unsubstituted, C₁₋₁₀₀₀ alkylene, substituted or unsubstituted, C₂₋₁₀₀₀ alkenylene, substituted or unsubstituted, C₂₋₁₀₀₀ alkynylene, substituted or unsubstituted, C₁₀₀₀ heteroalkylene, substituted or unsubstituted, C₂₋₁₀₀₀ heteroalkenylene, or substituted or unsubstituted, C₂₋₁₀₀₀ heteroalkynylene;
optionally wherein one or more (e.g., 2, 3, or 4) backbone carbon atoms of the C₁₋₁₀₀₀ alkylene, C₂₋₁₀₀₀ alkenylene, C₂₋₁₀₀₀ alkynylene, C₁₀₀₀ heteroalkylene, C₂₋₁₀₀₀ heteroalkenylene, or C₂₋₁₀₀₀ heteroalkynylene are independently replaced with substituted or unsubstituted carbocyclylene, substituted or unsubstituted heterocyclylene, substituted or unsubstituted arylene, or substituted or unsubstituted heteroarylene, as valency permits. In certain embodiments, the linker moiety is substituted or unsubstituted, C₂₀₋₄₀₀ alkylene, substituted or unsubstituted, C₂₀₋₄₀₀ alkenylene, substituted or unsubstituted, C₂₀₋₄₀₀ alkynylene, substituted or unsubstituted, C₂₀₋₄₀₀ heteroalkylene, substituted or unsubstituted, C₂₀₋₄₀₀ heteroalkenylene, or substituted or unsubstituted, C₂₀₋₄₀₀ heteroalkynylene;
optionally wherein one or more (e.g., 2, 3, or 4) backbone carbon atoms of the C₂₀₋₄₀₀ alkylene, C₂₀₋₄₀₀ alkenylene, C₂₀₋₄₀₀ alkynylene, C₂₀₋₄₀₀ heteroalkylene, C₂₀₋₄₀₀ heteroalkenylene, or C₂₀₋₄₀₀ heteroalkynylene are independently replaced with substituted or unsubstituted carbocyclylene, substituted or unsubstituted heterocyclylene, substituted or unsubstituted arylene, or substituted or unsubstituted heteroarylene, as valency permits.

In certain embodiments, the linker moiety is substituted or unsubstituted, C₂₀₋₄₀₀ alkylene or substituted or unsubstituted, C₂₀₋₄₀₀ heteroalkylene.

In certain embodiments, the linker moiety is substituted or unsubstituted, C₁₋₁₀ alkylene or substituted or unsubstituted, C₁₋₁₀ heteroalkylene.

In certain embodiments, the linker moiety is substituted or unsubstituted, C₂₀₋₄₀₀ alkylene or substituted or unsubstituted, C₂₀₋₄₀₀ heteroalkylene, wherein one, two, or three backbone carbon atoms of the C₂₀₋₄₀₀ alkylene or C₂₀₋₄₀₀ heteroalkylene are independently replaced with substituted or unsubstituted carbocyclylene, substituted or unsubstituted heterocyclylene, substituted or unsubstituted arylene, or substituted or unsubstituted heteroarylene, as valency permits.

In certain embodiments, the linker moiety is substituted or unsubstituted, C₂₀₋₄₀₀ heteroalkylene; optionally wherein one, two, or three backbone carbon atoms of C₂₀₋₄₀₀ heteroalkylene are independently replaced with substituted or unsubstituted carbocyclylene, substituted or unsubstituted heterocyclylene, substituted or unsubstituted arylene, or substituted or unsubstituted heteroarylene, as valency permits.

In certain embodiments, the linker moiety is substituted or unsubstituted, C₂₀₋₄₀₀ heteroalkylene, wherein one or two backbone carbon atoms of the C₂₀₋₄₀₀ heteroalkylene are independently replaced with substituted or unsubstituted, 3- to 7-membered, monocyclic carbocyclylene, substituted or unsubstituted, 3- to 7-membered, monocyclic heterocyclylene, substituted or unsubstituted phenylene, or substituted or unsubstituted, 5- or 6-membered, monocyclic heteroarylene, as valency permits.

In certain embodiments, the linker moiety is substituted or unsubstituted, C₂₀₋₄₀₀ heteroalkylene; -(substituted or unsubstituted heterocyclylene)-(substituted or unsubstituted, C₂₀₋₄₀₀ heteroalkylene)-; or -(substituted or unsubstituted, C₂₀₋₄₀₀ heteroalkylene)-(substituted or unsubstituted heterocyclylene)-.

In certain embodiments, the linker moiety is -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁; -(substituted or unsubstituted heterocyclylene)-(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-; or -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-(substituted or unsubstituted heterocyclylene)-.

In certain embodiments, the linker moiety is -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁; -(substituted or unsubstituted, 3- to 7-membered, monocyclic heterocyclylene)-(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-; or -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-(substituted or unsubstituted, 3- to 7-membered, monocyclic heterocyclylene)-.

In certain embodiments, the linker moiety is substituted or unsubstituted, C₂₋₁₀₀₀ (e.g., C₂₀₋₄₀₀)alkylene or substituted or unsubstituted, C₂₋₁₀₀₀ (e.g., C₂₀₋₄₀₀) heteroalkylene, wherein one or more (e.g., 2 or 3) backbone carbon atoms of the C₂₋₁₀₀₀ (e.g., C₂₀₋₄₀₀) alkylene or C₂₋₁₀₀₀ (e.g., C₂₀₋₄₀₀) heteroalkylene are independently replaced with a moiety formed by reacting a click-chemistry handle with its orthogonal click-chemistry handle through click chemistry

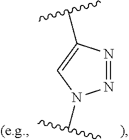 [SMILES: *c1cn(*)nn1]

wherein the attachment points are at either direction.

Other linkers suitable for use in the disclosed technology include, but are not limited to, ‘rigid’ linkers, such as those found in viral coat proteins, for example, (EAAAK)₁₋₃. This is an example of an alpha helix-forming linker used in many recombinant fusion proteins. Another example of a suitable rigid linker is PAPAP, or (Ala-Pro)ₙ (10-34aa), which is an example of a proline-rich ‘rigid’ linker where the accompanying aa can be any residue, but preferably is Ala, Lys and/or Glu.

Another examples of linkers that may be useful for the disclosed technology are cleavable linkers. For example, PLG*LWA where * is a cleavage site sensitive to metalloproteases, or GFLG*where* is a furin sensitive cleavage site. An alternative cleavable linker suitable for use in the disclosed technology is a disulfide bond that once reduced results in two separate compounds that were previously linked by a disulfide bond.

Another examples of linkers that may be useful for the disclosed technology are linkers comprising a moiety formed by reacting a click-chemistry handle with its orthogonal click-chemistry handle through click chemistry.

In particular embodiments of the present technology, the linker group is or comprises Gly-Gly-Gly-Ser (GGGS; SEQ ID No. 35). Optionally, the linker group may comprise an additional terminal residue to help facilitate attaching a desired therapeutic moiety. In some embodiments, the terminal residue may be Cys or Lys. In certain embodiments, such a linker group may be Gly-Gly-Gly-Ser-Cys (GGGSC; SEQ ID No. 52) or Gly-Gly-Gly-Ser-Lys (GGGSK; SEQ ID No. 53). In particular embodiments, the peptide may have a structure according to Formula II:

H—X₁—X₂—(X₃)ₙ—X₄—X₅—X₆—X₇-GGGS-X₈ Formula II.

With respect to Formula II, X₁, X₂, X₃, X₄, X₅, X₆, X₇ and n are as described herein (e.g., previously defined), and X₈ is H, Cys or Lys.

In certain embodiments, the linker moiety is of the formula:

—(Y₁)ₘ—Y₂,

wherein:
each Y₁ is independently an amino acid;
m is 2, 3, 4, 5, 6, 7, 8, 9, or 10; and
Y₂ is Cys, Ser, or Lys, optionally protected with an oxygen protecting group.

In certain embodiments, each Y₁ is independently a canonical amino acid. In certain embodiments, each Y₁ is independently Gly, Pro, Ala, Val, Ile, Leu, Met, Phe, Ser, Thr, Tyr, or Trp. In certain embodiments, each Y₁ is independently Gly or Ser.

In certain embodiments, m is 3. In certain embodiments, m is 4. In certain embodiments, m is 5.

In certain embodiments, Y₂ is Cys.

In certain embodiments, the linker moiety has a sequence -Gly-Gly-Gly-Ser-Cys (SEQ ID No. 52).

In certain embodiments, the peptide is of the sequence:
Leu-Ala-Phe-His-Arg-Met-Pro-Gly-Gly-Gly-Ser-Cys (SEQ ID NO: 54);
His-Leu-Ser-Ser-Leu-Thr-Pro-Gly-Gly-Gly-Ser-Cys (SEQ ID NO: 55); or
Ala-Thr-Gly-Pro-Arg-Ser-Val-Gly-Gly-Gly-Ser-Cys (SEQ ID NO: 56).

In some embodiments, the peptide according to Formula II is selected from:
Ser-Pro-Ala-Leu-His-Phe-Leu-Gly-Gly-Gly-Ser-Cys (SEQ ID No. 32),
Ser-Asn-Leu-Ala-Ala-Phe-Pro-Gly-Gly-Gly-Ser-Cys (SEQ ID No. 33) or
Met-Pro-Val-Ala-Val-Tyr-Arg-Gly-Gly-Gly-Ser-Cys (SEQ ID No. 34).

In alternative embodiments of Formula I, each of R¹ and R² is Cys, thereby forming a peptide having a structure according to Formula III:

Cys-X₁—X₂—(X₃)ₙ—X₄—X₅—X₆—X₇-Cys Formula III.

With respect to Formula III, X₁, X₂, X₃, X₄, X₅, X₆, X₇ and n are as described herein (e.g., previously defined). Also, with respect to Formula III, the two terminal Cys residues may form a disulfide bond, to form a circular peptide having a Formula IV.

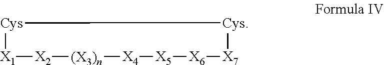

With respect to Formula IV, X₁, X₂, X₃, X₄, X₅, X₆, X₇ and n are as described herein (e.g., previously defined).

With respect to Formulas III and IV, a person of ordinary skill in the art understands that a linking moiety (linker moiety), such as GGGCS, can be attached to the peptide as described above with respect to Formulas I and II to further attach a desired agent to the peptide. In certain embodiments, the linker moiety is covalently attached (e.g., at the N-terminus) to the C-terminus of the peptide. In certain embodiments, the linking moiety is covalently attached (e.g., at the C-terminus) to the N-terminus of the peptide.

In other embodiments, R² is OH or a linker moiety, R¹ is Cys, and the peptide comprises an additional Cys attached to X₆; R¹ is H or a linker moiety, R² is Cys and the peptide comprises an additional Cys attached to X₂; or R¹ is H or a linker moiety, R² is OH or a linker moiety, and the peptide comprises additional Cys residues attached to X₂ and X₆. In some embodiments, the peptide may have a structure according to any one of Formulas IV-A, IV-B, or IV-C:

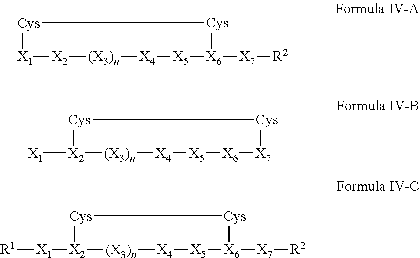

And with respect to any embodiments of Formulas I to IV-C, n may be 0, 1 or 2, and in some embodiments, n is 1.

In certain embodiments, each nitrogen protecting group is independently formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonoethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, phenacylsulfonamide, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salt, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fem), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidate, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, or 3-nitropyridinesulfenamide (Npys).

In certain embodiments, each oxygen protecting group is independently methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, or tosylate (Ts).

In another aspect, the present disclosure provides conjugates of the formula:

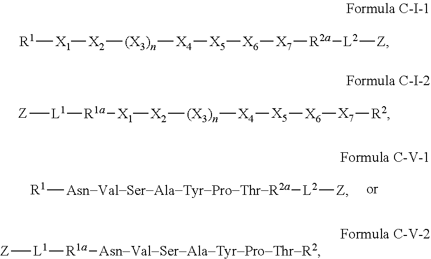

wherein:
R¹, X₁, X₂, X₃, n, X₄, X₅, X₆, X₇, and R² are as defined herein;
R^{1a} is a divalent radical formed by removing one hydrogen atom from R¹;
R^{2a} is a divalent radical formed by removing one hydrogen atom from R²;
each of L¹ and L² is a second linker moiety; and
Z is a lipid, small molecule, peptide, polypeptide, protein, nucleic acid, or saccharide.

In certain embodiments, the second linker moiety is substituted or unsubstituted, C₁₋₁₀₀₀ alkylene, substituted or unsubstituted, C₂₋₁₀₀₀ alkenylene, substituted or unsubstituted, C₂₋₁₀₀₀ alkynylene, substituted or unsubstituted, C₁₀₀₀ heteroalkylene, substituted or unsubstituted, C₂₋₁₀₀₀ heteroalkenylene, or substituted or unsubstituted, C₂₋₁₀₀₀ heteroalkynylene;
optionally wherein one or more (e.g., 2, 3, or 4) backbone carbon atoms of the C₁₋₁₀₀₀ alkylene, C₂₋₁₀₀₀ alkenylene, C₂₋₁₀₀₀ alkynylene, C₁₀₀₀ heteroalkylene, C₂₋₁₀₀₀ heteroalkenylene, or C₂₋₁₀₀₀ heteroalkynylene are independently replaced with substituted or unsubstituted carbocyclylene, substituted or unsubstituted heterocyclylene, substituted or unsubstituted arylene, or substituted or unsubstituted heteroarylene, as valency permits. In certain embodiments, the second linker moiety is substituted or unsubstituted, C₂₀₋₄₀₀ alkylene, substituted or unsubstituted, C₂₀₋₄₀₀ alkenylene, substituted or unsubstituted, C₂₀₋₄₀₀ alkynylene, substituted or unsubstituted, C₂₀-400 heteroalkylene, substituted or unsubstituted, C₂₀₋₄₀₀ heteroalkenylene, or substituted or unsubstituted, C₂₀₋₄₀₀ heteroalkynylene;
optionally wherein one or more (e.g., 2, 3, or 4) backbone carbon atoms of the C₂₀₋₄₀₀ alkylene, C₂₀₋₄₀₀ alkenylene, C₂₀₋₄₀₀ alkynylene, C₂₀₋₄₀₀ heteroalkylene, C₂₀₋₄₀₀ heteroalkenylene, or C₂₀₋₄₀₀ heteroalkynylene are independently replaced with substituted or unsubstituted carbocyclylene, substituted or unsubstituted heterocyclylene, substituted or unsubstituted arylene, or substituted or unsubstituted heteroarylene, as valency permits.

In certain embodiments, the second linker moiety is substituted or unsubstituted, C₂₀₋₄₀₀ alkylene or substituted or unsubstituted, C₂₀₋₄₀₀ heteroalkylene.

In certain embodiments, the second linker moiety is substituted or unsubstituted, C₁₋₁₀ alkylene or substituted or unsubstituted, C₁₋₁₀ heteroalkylene.

In certain embodiments, the second linker moiety is substituted or unsubstituted, C₂₀₋₄₀₀ alkylene or substituted or unsubstituted, C₂₀₋₄₀₀ heteroalkylene, wherein one, two, or three backbone carbon atoms of the C₂₀₋₄₀₀ alkylene or C₂₀₋₄₀₀ heteroalkylene are independently replaced with substituted or unsubstituted carbocyclylene, substituted or unsubstituted heterocyclylene, substituted or unsubstituted arylene, or substituted or unsubstituted heteroarylene, as valency permits.

In certain embodiments, the second linker moiety is substituted or unsubstituted, C₂₀₋₄₀₀ heteroalkylene; optionally wherein one, two, or three backbone carbon atoms of C₂₀₋₄₀₀ heteroalkylene are independently replaced with substituted or unsubstituted carbocyclylene, substituted or unsubstituted heterocyclylene, substituted or unsubstituted arylene, or substituted or unsubstituted heteroarylene, as valency permits.

In certain embodiments, the second linker moiety is substituted or unsubstituted, C₂₀₋₄₀₀ heteroalkylene, wherein one or two backbone carbon atoms of the C₂₀₋₄₀₀ heteroalkylene are independently replaced with substituted or unsubstituted, 3- to 7-membered, monocyclic carbocyclylene, substituted or unsubstituted, 3- to 7-membered, monocyclic heterocyclylene, substituted or unsubstituted phenylene, or substituted or unsubstituted, 5- or 6-membered, monocyclic heteroarylene, as valency permits.

In certain embodiments, the second linker moiety is substituted or unsubstituted, C₂₀₋₄₀₀ heteroalkylene; -(substituted or unsubstituted heterocyclylene)-(substituted or unsubstituted, C₂₀-400 heteroalkylene)-; or -(substituted or unsubstituted, C₂₀₋₄₀₀ heteroalkylene)-(substituted or unsubstituted heterocyclylene)-.

In certain embodiments, the second linker moiety is -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁; -(substituted or unsubstituted heterocyclylene)-(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-; or -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-(substituted or unsubstituted heterocyclylene)-.

In certain embodiments, the second linker moiety is -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁; -(substituted or unsubstituted, 3- to 7-membered, monocyclic heterocyclylene)-(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-; or -(substituted or unsubstituted, C₁₋₁₀ alkylene or substituted or unsubstituted, C₁₋₁₀ heteroalkylene)₀₋₁-(PEG 500-8000)-(substituted or unsubstituted, C₁₋₁₀ alkylene or substituted or unsubstituted, C₁₋₁₀ heteroalkylene)₀₋₁-(substituted or unsubstituted, 3- to 7-membered, monocyclic heterocyclylene)-.

In certain embodiments, the PEG is a PEG 300-1000, PEG 1000-3000, PEG 3000-10000, or PEG 10000-30000. In certain embodiments, the PEG is a PEG 500-8000. In certain embodiments, the PEG 500-8000 is a PEG 1000-4000 (e.g., PEG 2000).

In certain embodiments, the second linker moiety comprises a moiety formed by reacting two orthogonal reaction handles. In certain embodiments, the two orthogonal reaction handles are:
—SH and a thiophile;
a Michael donor and a Michael acceptor;
a nucleophile and an electrophile; or
a click-chemistry handle and its orthogonal click-chemistry handle.

In certain embodiments, a thiophile is a Michael acceptor or electrophile. In certain embodiments, a Michael donor is a nucleophile. In certain embodiments, a Michael acceptor is substituted or unsubstituted, α,β-unsaturated carbonyl

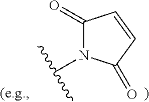 [SMILES: *N1C(=O)C=CC1=O]

or substituted or unsubstituted, α,β-γ,δ-diunsaturated carbonyl. In certain embodiments, a nucleophile is —SH, —OH, or —NH₂. In certain embodiments, an electrophile is —C(═O)-(a leaving group)

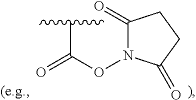 [SMILES: *C(=O)ON1C(=O)CCC1=O]

—S(═O)-(a leaving group), —S(═O)₂-(a leaving group) (e.g., —OS(═O)₂ (substituted or unsubstituted phenyl or substituted or unsubstituted, C₁₋₆ alkyl)), or —P(═O)(a leaving group)-. In certain embodiments, the click-chemistry handle is —N₃, substituted or unsubstituted nitrone, or substituted or unsubstituted 1,2,4,5-tetrazinyl. In certain embodiments, the orthogonal click-chemistry handle is —C≡CH, —C≡C— (e.g., substituted or unsubstituted, monocyclic, bicyclic, or tricyclic cycloalkynyl (e.g., substituted or unsubstituted, cyclooctynyl or azacyclooctynyl; or substituted or unsubstituted

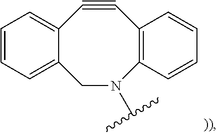 [SMILES: *N1Cc2ccccc2C#Cc2ccccc21]

or non-aromatic C═C (e.g., substituted or unsubstituted, monocyclic, bicyclic, or tricyclic, trans-cycloalkenyl (e.g., substituted or unsubstituted trans-cyclooctenyl)).

In certain embodiments, the moiety formed by reacting two orthogonal reaction handles is —C(═O)—S— or

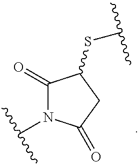 [SMILES: *SC1CC(=O)N(*)C1=O]

In certain embodiments, the moiety formed by reacting two orthogonal reaction handles is

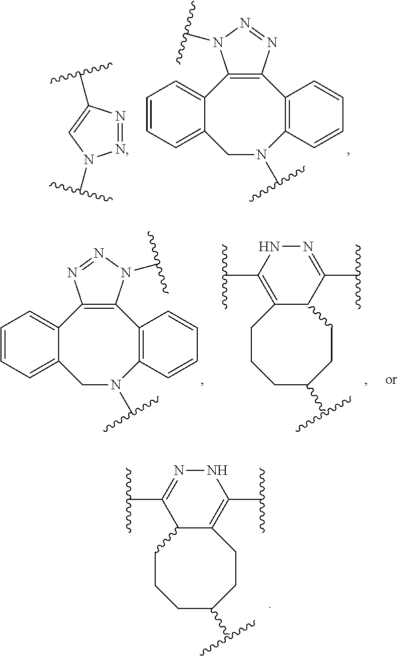

In certain embodiments, the second linker moiety is substituted or unsubstituted, C₂₋₁₀₀₀ (e.g., C₂₀₋₄₀₀) alkylene or substituted or unsubstituted, C₂₋₁₀₀₀ (e.g., C₂₀₋₄₀₀) heteroalkylene, wherein one or more (e.g., 2 or 3) backbone carbon atoms of the C₂₋₁₀₀₀ (e.g., C₂₀₋₄₀₀) alkylene or C₂₋₁₀₀₀ (e.g., C₂₀₋₄₀₀) heteroalkylene are independently replaced with a moiety formed by reacting a click-chemistry handle with its orthogonal click-chemistry handle through click chemistry

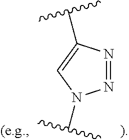 [SMILES: *c1cn(*)nn1]

In certain embodiments, Z is a lipid. In certain embodiments, Z is a lipid and is not a pharmaceutical agent.

In certain embodiments, Z is a small molecule, peptide, polypeptide, protein, nucleic acid, or saccharide. In certain embodiments, Z is a small molecule, peptide, polypeptide, protein, nucleic acid, or saccharide and is a pharmaceutical agent. In certain embodiments, Z is a small molecule. In certain embodiments, Z is a saccharide (e.g., a monosaccharide, disaccharide, oligosaccharide, or polysaccharide). In certain embodiments, Z is a peptide, polypeptide, or protein. In certain embodiments, Z is a nucleoprotein, mucoprotein, lipoprotein, glycoprotein, or protein conjugated with one or more small molecules. In certain embodiments, Z is a nucleic acid (e.g., DNA or RNA). In certain embodiments, Z is an mRNA. In certain embodiments, Z is an antibody, hormone, or vaccine. In certain embodiments, Z is an additional agent. In certain embodiments, Z is a small molecule, peptide, polypeptide, protein, nucleic acid, or saccharide, and is an additional agent. In certain embodiments, Z is a therapeutic agent for treating an eye disease. In certain embodiments, Z is an anti-angiogenic ophthalmic agent, miscellaneous ophthalmic agent, mydriatic, ophthalmic anesthetic, ophthalmic anti-infective, ophthalmic anti-inflammatory agent, ophthalmic antihistamines and decongestant, ophthalmic diagnostic agent, ophthalmic glaucoma agent, ophthalmic lubricants and irrigation, ophthalmic steroid, ophthalmic steroid with anti-infective, or ophthalmic surgical agent. In certain embodiments, Z is aflibercept, alcaftadine, apraclonidine, azelastine, azithromycin, bacitracin, bepotastine, besifloxacin, betaxolol, bimatoprost, brimonidine, brinzolamide, brolucizumab, bromfenac, ciprofloxacin, cyclopentolate, cyclosporine, dexamethasone, difluprednate, dorzolamide, echothiophate iodide, epinastine, faricimab, fluocinolone, fluorometholone, ganciclovir, gatifloxacin, homatropine, hydrocortisone, hydroxyamphetamine, ketorolac, ketotifen, latanoprost, latanoprostene bunod, levofloxacin, lifitegrast, lodoxamide, loteprednol, moxifloxacin, naphazoline, natamycin, nedocromil, neomycin, nepafenac, netarsudil, ocular lubricant, olopatadine, oxymetazoline, pegaptanib, pemirolast, pheniramine, phenylephrine, polymyxin b, prednisolone, ranibizumab, riboflavin, sulfacetamide sodium, tafluprost, tetrahydrozoline, timolol, tobramycin, travoprost, triamcinolone, trifluridine, trimethoprim, tropicamide, or zinc sulfate, or a pharmaceutically acceptable salt thereof; or a combination thereof. In certain embodiments, Z is an imaging agent. In certain embodiments, Z is a therapeutic agent, prophylactic agent, or diagnostic agent, approved by the US FDA or the European Medicines Agency for marketing.

### IV. Applications

### a) Compositions, Formulations, Kits, Methods of Making, and Methods of Use

The disclosed peptides may be used as targeting and/or delivery agents for applications where targeting the retina may be useful. The disclosed peptides may be attached to a desired moiety to be delivery to the retina though a suitable linking moiety, for example, Gly-Gly-Gly-Ser (GGGS; SEQ ID No. 35), Gly-Gly-Gly-Ser-Cys (GGGSC; SEQ ID No. 52) or Gly-Gly-Gly-Ser-Lys (GGGSK; SEQ ID No. 53). Moieties to be delivery to the retina include, but are not limited to, a therapeutic agent (for example, a nucleic acid, such as an antisense oligonucleotide (ASO) an RNA, such as, mRNA, siRNA, sgRNA/pegRNA or miRNA), an imaging agent (for example, a dye, fluorophore, radiotracer, radio-labelled peptide), or any combination thereof. The peptide may be directly to the agent(s) or it may be attached to a suitable carrier, such as a lipid nanoparticle, that provides the agent.

In certain embodiments, the composition comprises the peptide. In certain embodiments, the composition comprises the conjugate.

In certain embodiments, the composition further comprises a lipid (additional lipid).

In certain embodiments, the lipid is an ionizable lipid, a structural lipid, a polymer-conjugated lipid, or a combination thereof. In certain embodiments, the lipid is a combination of an ionizable lipid, and a polymer-conjugated lipid. In certain embodiments, the lipid is a combination of one or two ionizable lipids, and one or two polymer-conjugated lipids. In certain embodiments, the lipid is a combination of an ionizable lipid, a structural lipid, and a polymer-conjugated lipid. In certain embodiments, the lipid is a combination of one or two ionizable lipids, one or two structural lipids, and one or two polymer-conjugated lipids.

In certain embodiments, the lipid is an ionizable lipid. In certain embodiments, the ionizable lipid is a cationic lipid. In certain embodiments, the ionizable lipid comprises one or more (e.g., 2 or 3) basic nitrogen atoms. In certain embodiments, the ionizable lipid comprises only one basic nitrogen atom. In certain embodiments, the ionizable lipid is an anionic lipid. In certain embodiments, the ionizable lipid comprises one or more (e.g., 2 or 3) —O—P(═O)(OH)—O—, —O—P(═O)(O⁻)—O—, —C(═O)—OH, and/or —C(═O)—O⁻ moieties. In certain embodiments, the ionizable lipid comprises only one —O—P(═O)(OH)—O—, —O—P(═O)(O⁻)—O—, —C(═O)—OH, or —C(═O)—O⁻moiety. In certain embodiments, the ionizable lipid is a phospholipid. In certain embodiments, the ionizable lipid is a phospholipid or an ionizable lipid comprising one or more basic nitrogen atoms.

In certain embodiments, the phospholipid is 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or a combination thereof. In certain embodiments, the phospholipid is a glycerophospholipid. In certain embodiments, the phospholipid is a phosphatidic acid, phosphatidylethanolamine, phosphatidylcholine, phosphatidylserine, or phosphoinositide. In certain embodiments, the phospholipid is a phosphatidylinositol, phosphatidylinositol phosphate, phosphatidylinositol bisphosphate, or phosphatidylinositol trisphosphate. In certain embodiments, the phospholipid is a sphingolipid. In certain embodiments, the phospholipid is a ceramide phosphorylcholine, ceramide phosphorylethanolamine, or ceramide phosphoryllipid. In certain embodiments, the phospholipid is a phosphatidyl-ethanolamine (PE). In certain embodiments, the phospholipid is a phosphatidylcholine (PC).

In certain embodiments, the structural lipid is cholesterol, beta-sitosterol, fucosterol, campesterol, stigmastanol, or a combination thereof. In certain embodiments, the structural lipid is a sterol. In certain embodiments, the sterol is a phytosterol. In certain embodiments, the sterol is a zoosterol. In certain embodiments, the sterol is a cholesterol. In certain embodiments, the sterol is a campesterol, sitosterol (e.g., beta sitosterol), stigmasterol, or ergosterol. In certain embodiments, the sterol is a hopanoid, hydroxysteroid, or steroid.

In certain embodiments, the lipid is a polymer-conjugated lipid. In certain embodiments, the polymer portion of the polymer-conjugated lipid is a PEG. In certain embodiments, the polymer portion of the polymer-conjugated lipid is poly(N-(2-hydroxypropyl methacrylamide)), poly(vinyl pyrrolidone), poly(vinyl alcohol), polyglutamic acid, poly(malic acid), poly(amido amine), poly(ethyleneimine), poly(L-lysine), poly(L-glutamic acid), poly ((N-hydroxyalkyl)glutamine), dextrin, hydroxyethylstarch, polysialic acid, polyacetal (e.g., Fleximer®), poly(acrylamide), poly(methacrylic acid), poly(acrylic acid), poly(2-(dimethylamino)ethyl methacrylate, poly(N-isopropylacrylamide), poly((ethylene glycol)-co-N-(2-hydroxypropyl)methacrylamide)), or poly((ethylene glycol)-co-b-(ε-caprolactone)).

In certain embodiments, the lipid portion of the polymer-conjugated lipid is a fatty acid diglyceride. In certain embodiments, the fatty acid diglyceride is a C₆₋₁₀, C₁₁₋₁₅, C₁₆₋₂₀, C₂₁₋₂₅, or C₂₆₋₃₀, fatty acid diglyceride. In certain embodiments, the fatty acid diglyceride is a saturated or unsaturated (e.g., mono-, di-, or tri-unsaturated) fatty acid diglyceride. In certain embodiments, the fatty acid diglyceride is a linear or branched (e.g., mono-, di-, or tri-branched) fatty acid diglyceride. In certain embodiments, the fatty acid diglyceride is a fatty acid 1,2-diglyceride or fatty acid 1,3-diglyceride.

In certain embodiments, the polymer-conjugated lipid is a PEG-(fatty acid diglyceride). In certain embodiments, the polymer-conjugated lipid is dimyristoyl-rac-glycerol-PEG (DMG-PEG) (e.g., DMG-PEG 300-1000, DMG-PEG 1000-3000, or DMG-PEG 3000-10000 (e.g., DMG-PEG 2000)). In certain embodiments, the polymer-conjugated lipid is distearoyl-rac-glycerol-PEG (DSG-PEG) (e.g., DSG-PEG 300-1000, DSG-PEG 1000-3000, or DSG-PEG 3000-10000 (e.g., DSG-PEG 2000)).

In certain embodiments, the composition further comprises a particle. In certain embodiments, the particle is a nanoparticle. In certain embodiments, the particle is a microparticle. In certain embodiments, the composition comprises a micelle. In certain embodiments, the conjugate and additional lipid are part of the outer shell of the particle or micelle.

In certain embodiments, the composition further comprises an additional agent. In certain embodiments, the additional agent is encapsulated in the particle or micelle. In certain embodiments, the additional agent is non-covalently associated with the rest of the composition (e.g., conjugate).

In certain embodiments, the composition further comprises an excipient (e.g., pharmaceutically acceptable excipient).

In certain embodiments, the composition comprises:
the conjugate;
optionally an ionizable lipid;
optionally a polymer-conjugated lipid;
optionally a structural lipid;
optionally an additional agent; and
optionally a pharmaceutically acceptable excipient.

In certain embodiments, the composition comprises:
the conjugate;
an ionizable lipid;
a polymer-conjugated lipid;
a structural lipid;
optionally an additional agent; and
optionally a pharmaceutically acceptable excipient.

In certain embodiments, the composition comprises:
the conjugate;
an ionizable lipid;
a polymer-conjugated lipid;
a structural lipid; and
optionally a pharmaceutically acceptable excipient.

In certain embodiments, the composition comprises:
the conjugate;
an ionizable lipid;
a polymer-conjugated lipid;
a structural lipid;
an additional agent; and
optionally a pharmaceutically acceptable excipient.

In certain embodiments, the composition comprises:
the conjugate;
an ionizable lipid;
a polymer-conjugated lipid; and
optionally a pharmaceutically acceptable excipient.

In certain embodiments, the composition comprises:
the conjugate;
an ionizable lipid;
a polymer-conjugated lipid;
an additional agent; and
optionally a pharmaceutically acceptable excipient.

In certain embodiments, the composition comprises:
the conjugate;
(6Z,9Z,28Z,31Z)-heptatriacont6,9,28,31-tetraene-19-yl 4-(dimethylamino)butanoate;
1,2-distearoyl-sn-glycero-3-phosphocholine;
1,2-dimyristoyl-rac-glycero-3-methoxypolyethylene glycol-2000;
cholesterol;
optionally an additional agent; and
optionally a pharmaceutically acceptable excipient.

In certain embodiments, the molar ratio of (combined ionizable lipids):(combined polymer-conjugated lipids):(combined structural lipids) is (60±100%):(1.5±100%):(38.5±100%).

In certain embodiments, the molar ratio of (combined ionizable lipids):(combined polymer-conjugated lipids):(combined structural lipids) is (60±70%):(1.5±70%):(38.5±70%).

In certain embodiments, the molar ratio of (combined ionizable lipids):(combined polymer-conjugated lipids):(combined structural lipids) is (60±50%):(1.5±50%):(38.5±50%).

In certain embodiments, the molar ratio of (combined ionizable lipids):(combined polymer-conjugated lipids):(combined structural lipids) is (60±30%):(1.5±30%):(38.5±30%).

In certain embodiments, the molar ratio of (combined ionizable lipids):(combined polymer-conjugated lipids):(combined structural lipids) is (60±20%):(1.5±20%):(38.5±20%).

In certain embodiments, the molar ratio of (combined ionizable lipids):(combined lipids included in the conjugate):(combined structural lipids) is (60±100%):(1.5±100%):(38.5±100%).

In certain embodiments, the molar ratio of (combined ionizable lipids):(combined lipids included in the conjugate):(combined structural lipids) is (60±70%):(1.5±70%):(38.5±70%).

In certain embodiments, the molar ratio of (combined ionizable lipids):(combined lipids included in the conjugate):(combined structural lipids) is (60±50%):(1.5±50%):(38.5±50%).

In certain embodiments, the molar ratio of (combined ionizable lipids):(combined lipids included in the conjugate):(combined structural lipids) is (60±30%):(1.5±30%):(38.5±30%).

In certain embodiments, the molar ratio of (combined ionizable lipids):(combined lipids included in the conjugate):(combined structural lipids) is (60±20%):(1.5±20%):(38.5±20%).

In certain embodiments, the molar ratio of (combined ionizable lipids):(combined polymer-conjugated lipids and lipids included in the conjugate):(combined structural lipids) is (60±100%):(1.5±100%):(38.5±100%).

In certain embodiments, the molar ratio of (combined ionizable lipids):(combined polymer-conjugated lipids and lipids included in the conjugate):(combined structural lipids) is (60±70%):(1.5±70%):(38.5±70%).

In certain embodiments, the molar ratio of (combined ionizable lipids):(combined polymer-conjugated lipids and lipids included in the conjugate):(combined structural lipids) is (60±50%):(1.5±50%):(38.5±50%).

In certain embodiments, the molar ratio of (combined ionizable lipids):(combined polymer-conjugated lipids and lipids included in the conjugate):(combined structural lipids) is (60±30%):(1.5±30%):(38.5±30%).

In certain embodiments, the molar ratio of (combined ionizable lipids):(combined polymer-conjugated lipids and lipids included in the conjugate):(combined structural lipids) is (60±20%):(1.5±20%):(38.5±20%).

In certain embodiments, the combined molar amounts of (1) the PEG included in the conjugate and (2) the PEG included in the combined polymer-conjugated lipids are (1.5±100%)% (e.g., (1.5±70%)%, (1.5±50%)%, (1.5±30%)%, or (1.5±20%)%) of the combined molar amounts of all types of lipids included in the composition.

In certain embodiments, the molar ratio of the PEG included in the conjugate to the PEG included in the combined polymer-conjugated lipids is between 0.5:9.5 and 1:9, between 1:9 and 2:8, between 2:8 and 3:7, or between 3:7 and 4:6, inclusive.

In certain embodiments, the molar ratio of the PEG included in the conjugate to the PEG included in the combined polymer-conjugated lipids is between 4:6 and 5:5, between 5:5 and 6:4, between 6:4 and 7:3, or between 7:3 and 8:2, inclusive.

The disclosed peptides or compositions thereof may be administered by injection. Solutions can be prepared in water or saline, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection can include sterile aqueous solutions, dispersions, or sterile powders comprising the peptides or compositions thereof adapted for the extemporaneous preparation of sterile injectable solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions. Other suitable buffers include, but are not limited to, PBS, HEPES buffer and/or Tris.

In another aspect, the present disclosure provides methods of making the composition, the methods comprise:
providing a first solution comprising the peptide or conjugate;
providing a second solution; and
mixing the first and second solutions to form the composition.

In certain embodiments, the first solution comprises an organic solvent. In certain embodiments, the organic solvent is one single organic solvent. In certain embodiments, the organic solvent is a mixture of two or more (e.g., three) organic solvents (e.g., organic solvents described in this paragraph). In certain embodiments, the organic solvent is a protic solvent. In certain embodiments, the organic solvent is an alcohol solvent (e.g., ethanol or isopropanol, or a mixture thereof). In certain embodiments, the organic solvent is an aprotic solvent. In certain embodiments, the organic solvent is dimethyl sulfoxide, N,N-dimethylformamide, or N,N-dimethylacetamide, or a mixture thereof. In certain embodiments, the organic solvent is acetonitrile. In certain embodiments, the organic solvent is a ketone solvent (e.g., acetone or methyl ethyl ketone, or a mixture thereof). In certain embodiments, the organic solvent is an ether solvent (e.g., tetrahydrofuran, 2-methyltetrahydrofuran, or 1,4-dioxane, or a mixture thereof). In certain embodiments, the first solution is a mixture of the organic solvent and water.

In certain embodiments, the first solution further comprises a lipid.

In certain embodiments, the second solution is an aqueous buffer solution. In certain embodiments, the aqueous buffer solution is a aqueous citrate buffer solution, aqueous acetate buffer solution, or aqueous phosphate buffer solution. In certain embodiments, the aqueous buffer solution is an aqueous buffer solution of pH between 4 and 5, between 4.5 and 5.5, between 5 and 6, between 5.5 and 6.5, between 6 and 7, between 6.5 and 7.5, between 7 and 8, or between 7.5 and 8.5, inclusive.

In certain embodiments, the second solution comprises an additional agent.

In certain embodiments, the method further comprises:
providing a third solution comprising an additional agent; and
mixing the mixture with the third solution to form the composition.

In certain embodiments, the additional agent is not covalently attached to the peptide or conjugate.

Also disclosed herein are kits comprising:
the peptide, conjugate, or composition; and
instructions for using the peptide, conjugate, or composition.

In certain embodiments, the kit comprises a first container. In certain embodiments, the first container comprises the peptide, conjugate, or composition. In some embodiments, the kit further comprises a second container. In certain embodiments, the second container comprises the instructions. In certain embodiments, the instructions comprise information required by a regulatory agency, such as the US FDA or European Medicines Agency. In certain embodiments, the instructions comprise prescribing information. In certain embodiments, the instructions are for using the peptide, conjugate, or composition with a pharmaceutical agent. In certain embodiments, the kit comprises no pharmaceutical agents. In certain embodiments, the second container comprises the first container. In some embodiments, the kit further comprises a third container. In certain embodiments, the third container comprises the excipient. In certain embodiments, the kit comprises a pharmaceutical agent. In certain embodiments, the third container comprises the pharmaceutical agent. In certain embodiments, the second container comprises the third container. In certain embodiments, each of the first, second, and third containers is independently a vial, ampule, bottle, syringe, dispenser package, tube, or box.

In another aspect, the present disclosure provides methods comprising administering to a subject the peptide or composition.

In another aspect, the present disclosure provides methods comprising administering to a subject the conjugate.

In certain embodiments, the amount of the peptide, conjugate, or composition is an effective amount (e.g., an amount effective for a method or use described herein).

In another aspect, the present disclosure provides methods of delivering a pharmaceutical agent to a subject in need thereof comprising administering to or implanting in the subject in need thereof an effective amount of:
the peptide, conjugate, or composition; and
a pharmaceutical agent.

In certain embodiments, the pharmaceutical agent is delivered to the eye of the subject. In certain embodiments, the pharmaceutical agent is delivered to the retina of the subject. In certain embodiments, the pharmaceutical agent is delivered to a photoreceptor cell, retinal pigment epithelium cell, or Müller glia of the subject.

In another aspect, the present disclosure provides methods of treating a disease comprising administering to or implanting in a subject in need thereof an effective amount of:
the peptide, conjugate, or composition; and
a therapeutic agent.

In another aspect, the present disclosure provides methods of preventing a disease comprising administering to or implanting in a subject in need thereof an effective amount of:
the peptide, conjugate, or composition; and
a prophylactic agent.

In another aspect, the present disclosure provides methods of diagnosing a disease comprising administering to or implanting in a subject in need thereof an effective amount of:
the peptide, conjugate, or composition; and
a diagnostic agent.

In another aspect, the present disclosure provides uses of the peptides, conjugates, and compositions in the methods.

In another aspect, the present disclosure provides uses of the peptides, conjugates, and compositions in the manufacture of a medicament for use in the methods.

In another aspect, the present disclosure provides the peptides, conjugates, and compositions for use in the methods.

In certain embodiments, the pharmaceutical agent is a therapeutic agent. In certain embodiments, the pharmaceutical agent is a prophylactic agent. In certain embodiments, the pharmaceutical agent is a diagnostic agent. In certain embodiments, the additional agent, pharmaceutical agent, therapeutic agent, prophylactic agent, or diagnostic agent is a nucleic acid.

In certain embodiments, the nucleic acid comprises an antisense oligonucleotide (ASO), mRNA, siRNA, miRNA, or a combination thereof. In certain embodiments, the nucleic acid is an asymmetrical interfering RNA (aiRNA), Dicer-substrate RNA (dsRNA), small hairpin RNA (shRNA), transfer RNA (tRNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, viral DNA, circular RNA (circRNA), precursor messenger RNA (pre-mRNA), guide RNA (gRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, viral satellite RNA, or a combination thereof. In certain embodiments, the nucleic acid is an mRNA.

In certain embodiments, the therapeutic agent is a therapeutic agent for treating an eye disease. In certain embodiments, the therapeutic agent is an anti-angiogenic ophthalmic agent, miscellaneous ophthalmic agent, mydriatic, ophthalmic anesthetic, ophthalmic anti-infective, ophthalmic anti-inflammatory agent, ophthalmic antihistamines and decongestant, ophthalmic diagnostic agent, ophthalmic glaucoma agent, ophthalmic lubricants and irrigation, ophthalmic steroid, ophthalmic steroid with anti-infective, or ophthalmic surgical agent. In certain embodiments, the therapeutic agent is aflibercept, alcaftadine, apraclonidine, azelastine, azithromycin, bacitracin, bepotastine, besifloxacin, betaxolol, bimatoprost, brimonidine, brinzolamide, brolucizumab, bromfenac, ciprofloxacin, cyclopentolate, cyclosporine, dexamethasone, difluprednate, dorzolamide, echothiophate iodide, epinastine, faricimab, fluocinolone, fluorometholone, ganciclovir, gatifloxacin, homatropine, hydrocortisone, hydroxyamphetamine, ketorolac, ketotifen, latanoprost, latanoprostene bunod, levofloxacin, lifitegrast, lodoxamide, loteprednol, moxifloxacin, naphazoline, natamycin, nedocromil, neomycin, nepafenac, netarsudil, ocular lubricant, olopatadine, oxymetazoline, pegaptanib, pemirolast, pheniramine, phenylephrine, polymyxin b, prednisolone, ranibizumab, riboflavin, sulfacetamide sodium, tafluprost, tetrahydrozoline, timolol, tobramycin, travoprost, triamcinolone, trifluridine, trimethoprim, tropicamide, or zinc sulfate, or a pharmaceutically acceptable salt thereof; or a combination thereof.

In certain embodiments, the pharmaceutical agent, therapeutic agent, prophylactic agent, diagnostic agent, or additional agent is a small molecule, protein, polypeptide, antibody, peptide, or a combination thereof. In certain embodiments, the additional agent, pharmaceutical agent, therapeutic agent, prophylactic agent, or diagnostic agent is a small molecule, peptide, polypeptide, protein, nucleic acid, or saccharide. In certain embodiments, the additional agent, pharmaceutical agent, therapeutic agent, prophylactic agent, or diagnostic agent is a small molecule. In certain embodiments, the additional agent, pharmaceutical agent, therapeutic agent, prophylactic agent, or diagnostic agent is a saccharide (e.g., a monosaccharide, disaccharide, oligosaccharide, or polysaccharide). In certain embodiments, the additional agent, pharmaceutical agent, therapeutic agent, prophylactic agent, or diagnostic agent is a peptide, polypeptide, or protein. In certain embodiments, the additional agent, pharmaceutical agent, therapeutic agent, prophylactic agent, or diagnostic agent is a nucleoprotein, mucoprotein, lipoprotein, glycoprotein, or protein conjugated with one or more small molecules. In certain embodiments, the pharmaceutical agent, diagnostic agent, or additional agent is an imaging agent.

In certain embodiments, the pharmaceutical agent, therapeutic agent, prophylactic agent, diagnostic agent, or additional agent is approved by the US FDA or the European Medicines Agency for marketing.

In certain embodiments, the disease is a genetic disease, neoplasm, inflammatory disease, autoimmune disease, hematological disease, neurological disease, painful condition, metabolic disease, or psychiatric disorder. In certain embodiments, the disease is an eye disease. In certain embodiments, the eye disease is Aicardi syndrome, allergic eye disease, aphakia, autoimmune eye disease, benign mucous membrane pemphigoid, blepharophimosis syndrome, blindness, buphthalmia, CHARGE syndrome, cataract, chalazion, choroid disease, Cogan syndrome, Cohen syndrome, conjunctiva disease, corneal disease, cycloplegia, dry eye syndrome, Duane syndrome, ectopia lentis, exophthalmos, eye hemorrhage, eye infection, eye injury, eye irritation, eye lesion, eye neoplasm, eye pain, eyelid disease, Fuchs heterochromic iridocyclitis, glaucoma, Harada syndrome, incontinentia pigmenti, iridopathy, Jalili syndrome, keratoconus, lacrimal gland disease, Leber congenital amaurosis, Mainzer-Saldino syndrome, Miller Fisher syndrome, miosis, mydriasis, night blindness, nystagmus, ocular albinism, ocular edema, ocular fibrosis, ocular hypertension, ocular hypotension, ocular ischemia, ocular neovascularization, ocular photophobia, ocular rosacea, ocular synechia, oculoauriculovertebral dysplasia, oculopharyngeal muscular dystrophy, ophthalmia, ophthalmitis, ophthalmoplegia, opsoclonus, optic atrophy, osteoporosis-pseudoglioma syndrome, paraneoplastic ocular syndrome, Peters anomaly, phlyctenule, posterior capsule opacification, pseudoinflammatory fundus dystrophy, pseudophakia, pupil disease, retinal disease, Rieger syndrome, Rothmund-Thomson syndrome, Senior-Loken syndrome, Sjogren syndrome, Sorsby fundus dystrophy, tonic pupil, or uveal disease. In certain embodiments, the disease is acanthamoeba keratitis, allergic conjunctivitis, allergic rhinoconjunctivitis, anterior corneal dystrophy, autoimmune uveitis, autoimmune uveoretinitis, autosomal dominant neovascular inflammatory vitreoretinopathy, Bietti crystalline corneoretinal dystrophy, Blau syndrome, chorioretinal gyrate atrophy, chorioretinitis, choroid melanoma, choroideremia, congenital stationary night blindness, corneal arcus juvenilis, corneal epithelium dystrophy, corneal epithelium injury, corneal granular dystrophy, corneal lattice dystrophy, corneal perforation, diabetic maculopathy, episcleritis, eye squamous cell carcinoma, Fuchs heterochromic iridocyclitis, gelatinous drop-like corneal dystrophy, harderian gland neoplasm, herpetic keratitis, Kearns-Sayre syndrome, keratoconjunctivitis, macular corneal dystrophy, macular degeneration, macular dystrophy, macular edema, macular hole, microphthalmia with linear skin defects syndrome, panuveitis, polypoidal choroidal vasculopathy, posterior corneal dystrophy, proliferative vitreoretinopathy, retina ganglion cell injury, retinal dialysis, retinal photoreceptor degeneration, retinitis pigmentosa, retinoblastoma, seasonal allergic rhinoconjunctivitis, Sengers syndrome, serpiginous choroiditis, stromal corneal dystrophy, uveal melanoma, uveoparotid fever, WAGR syndrome, or X-linked megalocornea. In certain embodiments, the disease is age-related macular degeneration, autosomal dominant retinitis pigmentosa, autosomal recessive retinitis pigmentosa, Avellino corneal dystrophy, best vitelliform macular dystrophy, choroid melanoma, congenital hereditary endothelial corneal dystrophy, cystoid macular edema, diabetic macular edema, epithelial basement membrane corneal dystrophy, Fuchs endothelial dystrophy, harderian gland carcinoma, Lisch corneal dystrophy, Meesmann corneal dystrophy, neurogenic ataxia and retinitis pigmentosa syndrome, posterior polymorphous corneal dystrophy, Reis-Bucklers corneal dystrophy, Schnyder crystalline corneal dystrophy, seasonal allergic rhinoconjunctivitis, Stargardt disease, superior limbic keratoconjunctivitis, Thiel-Behnke corneal dystrophy, Usher syndrome, X-linked endothelial corneal dystrophy, or X-linked retinitis pigmentosa.

In certain embodiments, the disease is a retinal disease. In certain embodiments, the retinal disease is Aicardi syndrome, angioid streak, central serous chorioretinopathy, chorioretinal disease, Coats disease, congenital stationary night blindness, diabetic retinopathy, Eales disease, familial exudative vitreoretinopathy, hypertensive retinopathy, inherited retinal disease, maculopathy, proliferative retinopathy, retinal artery occlusion, retinal cone cell dystrophy, retinal degeneration, retinal detachment, retinal drusen, retinal dystrophy, retinal hemorrhage, retinal injury, retinal ischemia, Retinal neoplasm, retinal neovascularization, retinal tear, retinal thrombosis, retinal vascular disease, retinal vein occlusion, retinitis, retinopathy of prematurity, or X-linked juvenile retinoschisis. In certain embodiments, the retinal disease is autosomal dominant neovascular inflammatory vitreoretinopathy, Bietti crystalline corneoretinal dystrophy, chorioretinal gyrate atrophy, chorioretinitis, choroideremia, diabetic maculopathy, macular degeneration, macular dystrophy, macular edema, macular hole, polypoidal choroidal vasculopathy, proliferative vitreoretinopathy, retina ganglion cell injury, retinal dialysis, retinal photoreceptor degeneration, retinitis pigmentosa, or retinoblastoma.

In certain embodiments, the subject is a human. In certain embodiments, the subject is a human aged less than 1 month. In certain embodiments, the subject is a human aged 1 month to less than 2 years. In certain embodiments, the subject is a human aged 2 to less than 12 years. In certain embodiments, the subject is a human aged 12 to less than 17 years. In certain embodiments, the subject is a human aged 17 or above.

In certain embodiments, the pharmaceutical agent is delivered to the eye of the subject. In certain embodiments, the pharmaceutical agent is delivered to the retina of the subject. In certain embodiments, the pharmaceutical agent is delivered to a photoreceptor cell, retinal pigment epithelia cell, or Müller glia of the eye of the subject.

### b) Dosage and Delivery Route

The peptides, conjugates, and compositions described herein may be delivered or administered to a subject by any suitable route. The disclosed composition comprising the disclosed peptide may be delivered to a subject by any suitable route, such as by injection (intravitreal or subretinal). In certain embodiments, the suitable route is by injection (e.g., injection into the eye). In certain embodiments, the suitable route is intravitreal, subretinal, subconjunctival, intracameral, subtenon, suprachoroidal, or posterior juxtascleral. In certain embodiments, the injection comprises intravitreal or subretinal injection. In certain embodiments, the suitable route is topical (e.g., as drops, ointments, pastes, creams, lotions, gels, powders, solutions, sprays, or patches). In certain embodiments, administering comprises administering topically.

Initial dosages can also be estimated from in vivo data, such as animal models. For dosage estimation for human administration, suitable animal models may either be animals selected or genetically modified to be susceptible diseases and/or conditions comparable to human diseases or conditions. Additionally, or alternatively, dosages can be estimated from administration to animals having an eye infection. Persons of ordinary skill in the art can adapt such information to determine dosages suitable for human administration.

### c) Additional Therapies

The disclosed peptide(s) or pharmaceutical compositions thereof, may be administered alone or in combination with one or more additional therapies. Suitable additional therapies include any therapy that may be administered to a subject's eye to treat one or more underlying conditions, or to ameliorate one or more symptoms of a disease or condition. In some embodiments, the disclosed peptide(s) or pharmaceutical compositions thereof, are administered in combination with, but are not limited to, an antibiotic, anti-inflammatory agent (such as a steroidal anti-inflammatory agent or a nonsteroidal anti-inflammatory agent), analgesic, antiviral, antibody, or a combination thereof.

### V. Examples

Lipid nanoparticles (LNPs) based mRNA delivery holds promise for the treatment of inherited retinal degenerations (IRDs). Currently, LNP mediated mRNA delivery is restricted to the retinal pigment epithelium (RPE) and Müller glia. LNPs must overcome ocular barriers to transfect neuronal cells critical for visual phototransduction—the photoreceptors (PRs). A combinatorial M13-bacteriophage heptameric peptide phage display library was utilized for the mining of peptide ligands that target PRs. The most promising peptide candidates resulting from in vivo biopanning were identified. Dye-conjugated peptides showed rapid localization to the PRs. LNPs decorated with the top-performing peptide ligands, delivered mRNA to the PRs, RPE and Müller glia in mice. This distribution translated to the non-human primate eye, wherein robust protein expression was observed in the PRs, Müller glia, and RPE. Overall, peptide conjugated LNPs have been developed that can enable mRNA delivery to the neural retina, expanding the utility of LNP-mRNA therapies for inherited blindness.

Lipid nanoparticles (LNPs) are the most clinically advanced non-viral platform for mRNA delivery. Their worldwide dissemination as part of the COVID-19 vaccine has proven their safety and efficacy. The inventors have focused on utilizing LNP mRNA delivery for the treatment of inherited retinal degenerations (IRDs). IRDs are a complex group of genetic disorders that arise from mutations in the over 300 different genes associated with retinal pathology. These genes are important for photoreceptor (PR) and retinal pigment epithelium (RPE) function and when compromised, progressive cell death leads to blindness.

Gene augmentation, editing and silencing are attractive forms of clinical care for these patients as they correct the causative genomic malfunction. Luxturna™, which utilizes an adeno-associated virus (AAV2) to deliver a normal copy of the RPE65 gene to the RPE, is FDA approved for biallelic RPE65 Leber congenital amaurosis patients. Luxturna™ has established the safety and efficacy of gene augmentation via subretinal delivery, and now many different AAV gene therapies are under pre-clinical and/or clinical development. In addition to subretinal administration, intravitreal delivery of AAVs, for X-linked retinoschisis, Leber hereditary optic neuropathy, and mutation-independent optogenetic strategies, is also demonstrating safety and efficacy in various clinic trials. AAVs have facilitated the advancement of gene editing in the retina as the first in-vivo CRISPR genome editing medicine, EDIT-101, was administered to CEP290 Leber congenital amaurosis patients. While both subretinal and intravitreal AAV gene therapy strategies are benefiting patients, AAVs have three main limitations; including limited DNA packaging capacity (<5 kb), immunogenicity, and the ability to constitutively express Cas9 nucleases, which highlight the need to develop novel gene delivery vehicles for the retina.

LNPs have a well-established safety profile in the clinic. These modular systems may encapsulate large size cargos, and their synthetic biodegradable chemistries alleviate the pressures of neutralizing antibodies and sustained immune responses. Ideally, gene editors would be delivered in the form of mRNA, allowing for robust and transient expression of nucleases, mitigating off-target effects. LNPs may deliver mRNA-based cargo that may lead to rapid protein production, in hard-to-transfect non-dividing cells with no risk of genomic integration. However, prior to replacing their viral counterparts, LNPs must be able to transfect the neuronal cells, which harbor many of the mutations associated with IRDs. The inventors' previous work demonstrated that regardless of compositional modifications, protein expression in the retina was predominately restricted to the phagocytic RPE cells and Müller glia. Either through subretinal or intravitreal delivery, LNPs have been unable to penetrate the neural retina, limiting their ability to deliver genes or gene editors to one of the most important cellular targets, the PRs.

The inventors postulated that chemically decorating LNPs with a short 7-mer peptide would allow them to permeate into the neural retina. Peptides are sequences of amino acids with varying lengths that can be naturally occurring or chemically synthesized. A given peptide chain may fulfill both a structural or bioactive role depending on charge densities, hydrophobicity, hydrophilicity, structural conformations and chemical modifications. By crossing biological barriers, peptides have been able to enhance drug delivery, imaging agents and nanoparticle drug targeting. A diverse M13-bacteriophage-based, heptameric peptide library was used to identify peptide sequences that bind the neural retina in vivo. Then, chemically-synthesized, peptide conjugates of these hits were used to confirm localization to the cell of interest. Decoration of these peptides on the surface of LNPs with varying surface densities resulted in successful delivery of mRNA to the neural retina in a mouse model. These results translated to the more clinically relevant non-human primate (NHP), where robust protein expression was observed in the PRs, Müller glia, and RPE. Overall, novel, peptide conjugated LNPs were identified that can facilitate the delivery of mRNA to the neural retina, expanding the utility of LNP-mRNA therapies for inherited blindness.

### MATERIALS AND METHODS

LNP and mRNA materials Cre recombinase (Cre) and GFP mRNA fully substituted with 5-methoxyuridine was purchased from Trilink Biotechnologies (L-7211 and L7203, respectively). (6Z,9Z,28Z,31Z)-heptatriacont6,9,28,31-tetraene-19-yl 4-(dimethylamino)butanoate (DLin-MC3-DMA) was custom synthesized by Biofine International, Inc (BC, Canada). 1,2-distearoyl-sn-glycero-3-phosphocholine (DSPC) was purchased from Avanti Polar Lipids, Inc (Alabaster, AL). 1,2-Dimyristoyl-rac-glycero-3-methoxypolyethylene glycol-2000 (DMG-PEG2K), Cholesterol was obtained from Sigma Aldrich (St Louis, MO). For peptide-conjugated formulations DMG-PEG2K was substituted with 1,2-distearoyl-sn-glycero-3-phosphoethanolamine-N-[maleimide(polyethylene glycol)-2000] (DSPE-PEG-Maleimide) for Ai9 mice and 1,2-distearoyl-sn-glycero-3-phosphoethanolamine-N-[carboxy(polyethylene glycol)-2000, NHS ester for NHP.

### LNP Formulation, Conjugation and Characterization

Lipid nanoparticles (LNPs) were formulated by a microfluidic mixing method. In short, ethanol solutions containing DLin-MC3-DMA, Cholesterol, DSPC, and DMG-PEG2K, at molar ratios of 50:38.5:10:1.5, were combined with 50 mM citrate buffer containing mRNA using a microfluidic mixer at a 1:3 ratio. LNPs were dialyzed two times using phosphate buffered saline (PBS, pH 7.4) and concentrated with 10 kDa Amicon® Ultra centrifuge filters (Millipore, Burlington, MA). For peptide-conjugated LNPs, DSPE-PEG-Maleimide- and DSPE-PEG-Carboxy-NHS-functionalized LNPs were incubated at 4° C. overnight in PBS with peptide ligands at molar excess of 10:1 (peptide:PEG) and subsequently concentrated via centrifugation. Size distribution and PDI of LNPs were determined via dynamic light scattering using a Zetasizer Nano ZSP (Malvern Instruments, UK) and Nanoparticle Tracking Analysis (NTA) was done using ZetaView TWIN equipped with video microscope PMX-220 (Ammersee, Germany). mRNA encapsulation efficiency was determined using Quant-iT RiboGreen RNA reagent before and after peptide conjugation (Invitrogen, Carlsbad, CA). Conjugated peptide ligand concentration was determined using fluorometric maleimide assay kit (Sigma-Aldrich) Cat. MAK167 as per manufacture's protocol. This assay was utilized to measure the peptide content on DSPE-PEG-Maleimide-LNPs.

### Cell Culture.

Cell culture media and reagents were purchased from Thermo Fisher Scientific (Waltham, MA). 661W cone cells were generously provided by Prof. Muayyad Al-Ubaidi, University of Houston, Houston, TX. Cells were cultured in DMEM high glucose (cat #11965175)+10% FBS+20 μg/mL progesterone and Hydro-21 Heme at 37° C., 5% CO₂, and split at a ratio of 1:6 once per week. hARPE19 cells (ATCC CRL-2302) were cultured in DMEM/F₁₂ (50:50 mix—cat #11320033)+10% FBS at 37° C., 5% CO₂, and split at a ratio of 1:10 once per week.

### Mouse Models

Albino BALB/c mice were purchased from The Jackson Laboratory (Bar Harbor, ME, USA). Male and female mice aged 2 to 4 months were used in experiments. All the experimental procedures followed the protocols approved by the Institutional Animal Care and Use Committee at Oregon Health & Science University and were in adherence to the Association for Research in Vision and Ophthalmology (ARVO) Statement for the Use of Animals in Ophthalmic and Vision Research.

### Phage Library and Phage Reagents.

The Ph.D.-7 phage display peptide library was purchased from New England Biolabs (Ipswich, MA, USA). This pentavalent display library contained approximately 1×10¹³ pfu/mL phages with a clonal diversity of 1.28×10⁹ unique peptide sequences. All reagents necessary for phage display incubation, isolation, amplification and purification prepared as described in Ph.D.-7 manufacturer's manual. Empty M13K phage vector (NEB cat. #E8101) was used to produce M13 phage without any peptides displayed on surface and this was used as a negative control.

### ELISAs

M13 Phage coat protein Monoclonal Antibody (E1), Biotin conjugated (cat. #MA1-34468) and Poly-HRP Streptavidin secondary antibody (cat. #N200) were purchased from Thermo Fisher Scientific.

### Peptide Synthesis.

Peptides with added linker were conjugated with TAMRA fluorophore at the carboxy-terminal Cysteine's side chain. The peptides were labelled, synthesized and purified using FMOC solid-phase peptide synthesis and reverse-phase HPLC by Thermo Fisher Scientific. Peptides used have the following sequences: MH3—DGPPRKPGGGSC (SEQ ID No. 31), MH42-SPALHFLGGGSC (SEQ ID No. 32), MH43—SNLAAFPGGGSC (SEQ ID No. 33) and MH50-MPVAVYRGGGSC (SEQ ID No. 34).

### In Vitro Biopanning.

ARPE 19 cells and 661w cone cells were seeded in 24-well cell culture plates (Thermo Fisher cat. #FB012929) at a density of 10⁵ cells/well in 1 mL of full media one day prior to start of panning process. After overnight incubation, the cells were washed four times with 1 mL pre-chilled DMEM (FBS-free) at 4° C. and cells were subsequently blocked in 1 mL DMEM supplemented with 1% BSA for 1 hour at 4° C. with gentle rocking. Once blocking was finished, 10 phage library equivalents (1×10¹² PFU/mL), representing approximately 100 copies per individual phage, are added in 1 mL of blocking buffer and phage-cell mixture is incubated for 1 hour at 4° C. with gentle rocking. After 1 hour the wells were washed and aspirated ten times with 1 mL of 0.1% TBST to remove weakly bound phages. After washing, 200 μL of 0.2 M Glycine-HCl (pH 2.2) 1 mg/mL BSA, was added to the wells and incubated at 4° C. for 10 minutes with gentle rocking. After 10 minute incubation, the reaction was neutralized with 30 μL addition of 1 M Tris-HCl, pH 9.1. 10 μL of the resulting neutralized, eluted phage solution was used for phage titering while the rest was used for amplification in E. coli K12 ER2738 (NEB cat. #E4104) followed by phage purification as per manufacturer's instructions. Serial dilutions (10-10³-fold) of phages were prepared in Luria-Bertani medium. Single-stranded DNA was extracted and purified from individual phage clones picked from titering plates for sanger sequencing by capillary electrophoresis (Applied Biosystems 3730xl DNA Analyzer) to elucidate candidate peptides after each unamplified round (the sequencing primer, 5′-CCCTCATAGTTAGCGTAACG-3′ (96 g III) SEQ ID No. 36). An empty, M13 phage library was used as a negative control for the first round of panning.

### In Vivo Biopanning

Before intravitreal injections, mice were topically administered 0.5% proparacaine, 1% tropicamide, and 2.5% phenylephrine and anesthetized with ketamine (100 mg/kg)/xylazine (10 mg/kg). To begin injection, 2.5% hypromellose was placed over the eye and a 30-gauge needle was used to make an incision in the limbus. Going through the scleral incision in the limbus, using a Hamilton syringe with a 33-gauge 20° beveled needle, 1.5 μL of Ph.D.-7 Phage library (1×10¹¹ PFU/mL) was administered to the intravitreal space for each of the three panning rounds performed. An empty, M13 phage library was administered as a negative control for the first round of panning. A 2% fluorescein solution was added to the phage libraries to observe and confirm successful intravitreal delivery. Six hours after administration, the neural retina was extracted from mouse eyes and washed with 0.1% TBST ten times followed by 200 μL of 0.2 M Glycine-HCl (pH 2.2) 1 mg/mL BSA elution for 10 minutes at 4° C. with gentle rocking. 30 uL of 1 M Tris-HCl, pH 9.1 neutralization buffer was added to the solution. 10 μL of the resulting neutralized, eluted phage solution was used for phage titering while the rest was used for amplification in E. coli K12 ER2738 followed by phage purification according to manufacturer. Serial dilutions (e.g., 10-10³-fold) of phages were prepared in Luria-Bertani medium. Single-stranded DNA was extracted and purified from individual phage clones picked from titering plates for sanger sequencing by capillary electrophoresis (Applied Biosystems 3730xl DNA Analyzer) to elucidate candidate peptides after each unamplified round (the sequencing primer, 5′-CCCTCATAGTTAGCGTAACG-3′ (96 g III) SEQ ID No. 36).

### Heatmaps Normalized Occurrence (% Occurrence)

A python script was written to take in a ‘.txt’ file containing tab-delimited sample labels and the corresponding 7mer peptide. The script parses the input.txt file, counting the total number of peptide sequences and adds each sequence to a list. A 20×7 integer matrix was initialized with each element having a value of 0, the 20 rows corresponding to the 20 Amino Acids and the 7 columns corresponding to the positions in the 7mer. The list of sequences was then analyzed, each character in each sequence was checked against a list of the 1 letter AA codes to ensure it was a valid AA, if the check passed the element in the final 20×7 matrix corresponding to the AA of the character and the position the character in the sequence was iterated by 1. Once all sequences were analyzed the final 20×7 matrix contained counts of each amino acid at each position in the sequence. The counts were then divided by the total number of sequences to yield a “normalized occurrence” or % occurrence of amino acids in each position of the sequence. The 20×7 matrix of normalized occurrence values was written to an output.txt file, tab-delimited, where it was exported to prism and the heatmaps were then finalized. The script was run on all 3 rounds of in-vivo enriched phage.

### Cell ELISA

ARPE19 and 661w cells were seeded a day before the experiment on 96 well plates (10⁴ cells/well). On day of experiment, cells were first fixed in 4% paraformaldehyde solution at room temperature for 15 minutes. This was followed by three washes with PBS and then the cells were blocked for 30 minutes at room temperature with 1% BSA in PBS. Biotin-conjugated, mouse anti-M13 phage coat protein primary antibody was added (Thermo Fisher cat. #MA1-34468) at 1:5000 dilution in block buffer with 0.1% TBST and incubated for 2 hours at room temperature with gentle rocking. The wells were then washed with 0.1% TBST and individual phage clones were incubated for one hour at room temperature in blocking buffer with gentle rocking. After phage incubation, wells were washed three times with 0.1% TBST to reduce non-specific binding and eliminate weakly bound phages. Biotin-conjugated, mouse anti-M13 phage coat protein primary antibody was added (Thermo Fisher cat. #MA1-34468) at 1:5000 dilution in 0.1% TBST and incubated for 1 hour at room temperature with gentle rocking. The wells were washed three more times with 0.1% TBST and subsequently incubated with poly-HRP streptavidin secondary antibody (Thermo Fisher cat. #N200) at 1:10000 dilution in 0.1% TBST for 1 hour at room temperature. Wells were then washed three times with 0.1% TBST and 3,3′,5,5′-Tetramethylbenzidine (Thermo Fisher) was added to the wells and incubated at room temperature for 5-10 minutes and the reaction was stopped with 0.2 M Sulfuric Acid and absorbance was read at 450 nm using TECAN Infinite200 Pro spectrophotometer (Tecan Group Ltd., Switzerland).

### Cell Internalization and Image Analysis

Approximately 50,000 cells were seeded per well of 8-well μ-Slide (Ibidi, Fitchburg, WI) and incubated with 10 nM and 50 nM TAMRA-labeled peptides for 30 minutes at 37° C. followed by washing with PBS, fixation in 4% Paraformaldehyde for 10 mins at room temperature. Cells were then washed three times, DAPI stained and cover slipped for confocal microscopy imaging. Fluorescence confocal images of cell internalization studies were captured with the same exposure settings and were analyzed for fluorescence intensity using ImageJ (version 1.45; National Institutes of Health, Bethesda, MD). First, the hARPE or 661w cells were outlined and ImageJ calculated the pixel intensity of the different cell treatments with different peptides. At least three images were analyzed for each peptide tested.

### Structural Analysis Superposition of Peptides Using Molecular Operating Environment (MOE)

Using the molecular builder module in MOE (Chemical Computing Group, Quebec, Canada), the peptide structure was drawn. The peptide side chain, N-terminus, and the C-terminus were assigned appropriate charges (depending on their ionization state at physiological pH). The peptide was then energy minimized using the Assisted Model Building with Energy Refinement (AMBER) module the in MOE. The same process was repeated for all the peptides. The top peptide candidates were superposed using the default superposition module in MOE software.

### Pharmaceutically Relevant Physicochemical Properties of Peptides Isolated

The peptide database prepared in MOE software was exported as Schrödinger compatible file (.mae). The database was manually checked for structure correctness. Pharmaceutically relevant Absorption, Distribution, Metabolism, and Excretion (ADME) properties were calculated using QikProp module in the Schrodinger software (Schrödinger, Inc, New York, NY).

### Mouse Injections

For subretinal injections, mice were topically administered 0.5% proparacaine, 1% tropicamide, and 2.5% phenylephrine and anesthetized with ketamine (100 mg/kg)/xylazine (10 mg/kg). To initiate the injection, 2.5% hypromellose was used to cover the eye and a 30-gauge needle was used to make an incision in the limbus. A glass coverslip was then placed over the eye to allow for visualization of the retina. Going through the scleral incision in the limbus, using a Hamilton syringe with a 33-gauge blunt needle, 1 μL PBS, peptide, or LNP-Cre were delivered to the subretinal space. A 2% fluorescein solution was added to the PBS and LNPs so retinal detachment could be confirmed. For most injections, scleral incisions in the limbus were created nasally and PBS or LNPs were delivered temporally. Intravitreal injections were performed as previously described elsewhere (25). For LNP-Cre subretinal injections, 200 ng (1 μL, 200 ng/μL) was delivered. For intravitreal injections 1.1 μg (1.5 μL, 741 ng/μL) was injected.

In Vivo Validation with TAMRA-Labelled Peptides.

Top peptide candidates were synthesized and subsequently conjugated with 5-(and −6)-carboxytetramethylrhodamine, succinimidyl ester (TAMRA) (Thermo Fisher), at the carboxy Cysteine and injected either subretinally or intravitreally, into balb/c mice aged 2-3 months following the same procedure as described earlier for phage in vivo biopanning. Following peptide injection, ophthalmic fundus imaging was conducted to preemptively gauge the retina distribution of the top performing TAMRA-labelled peptide candidate injected. At specified time points, mouse eyes were enucleated and fixed using 4% paraformaldehyde in PBS overnight at 4° C. Eyes were cryopreserved in 30% sucrose solution for 2 hours prior to embedding in OCT media followed by cryo-sectioning and confocal imaging. Retinal cryo-sections were 12 μm thick and DAPI stained prior to confocal imaging done using TCS SP8 X (Leica Microsystems, Buffalo Grove, IL). Z-stacks (spanned 10 μm with 1 μm interval) were collected using a 40× objective, and maximum intensity projections were used for further analysis. Ophthalmic fundus imaging was conducted to preemptively gauge the retina distribution of the top performing TAMRA-labelled peptide candidate injected.

In Vivo Validation with LNP-Conjugated Peptides.

Ai9 mice were injected intravitreally and subretinally with LNPs and LNP-peptides loaded with Cre-recombinase mRNA. At 7 days post injection, ophthalmic fundus imaging was performed to observe in vivo tdtomato distribution in the retina. Mouse eyes were then enucleated and fixed using 4% paraformaldehyde in PBS overnight at 4° C. Eyes were cryopreserved in 30% sucrose solution for 2 hours prior to embedding in OCT media. Retinal cryo-sections were 12 μm thick. Slides were stain with antibodies specific for visual arrestin (rod and cone PRs, Cat #sc-166383, Santa Cruz Biotechnology, Dallas, TX), IBA-1 (microglia; Cat #019-19741 Wako Chemicals, Richmond, VA), and CD3 (T-cells; Cat #sc20047, Santa Cruz Biotechnology, Dallas, TX). Primary antibodies were used at a concentration of 1:100, 1:500 and 1:50, respectively. Detection of primary antibodies was achieved using Alexafluor secondary antibodies at a concentration of 1:300; 4′,6-diamidino-2-phenylindole (DAPI) was used as a nuclear marker counterstain. Confocal imaging was performed using TCS SP8 X (Leica Microsystems, Buffalo Grove, IL). Z-stacks (spanned 10 μm with 1 μm interval) were collected using a 40× objective, and maximum intensity projections were used for further analysis. Slides were additionally stained with hematoxylin-eosin (H & E), and viewed on a Leica DMI3000 B microscope (Leica Microsystems GmbH, Wetzlar, Germany). All images were taken at a magnification of ×10.

### Fundus Imaging

In vivo retinal imaging was performed with the Micron IV (Phoenix Research Laboratories, Pleasanton, CA). To observe general retinal morphology, bright field images were acquired. To capture tdTomato, a 534/42 nm BrightLine® single-band bandpass filter (Semrock, Rochester, NY) was used. Light intensity, exposure and gain were kept consistent across all RFP images.

### Confocal Microscopy

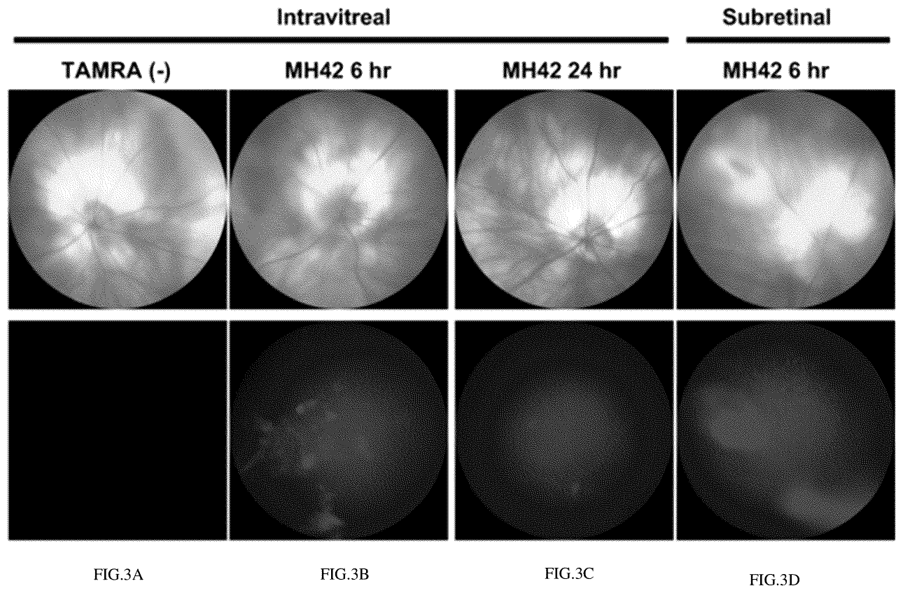

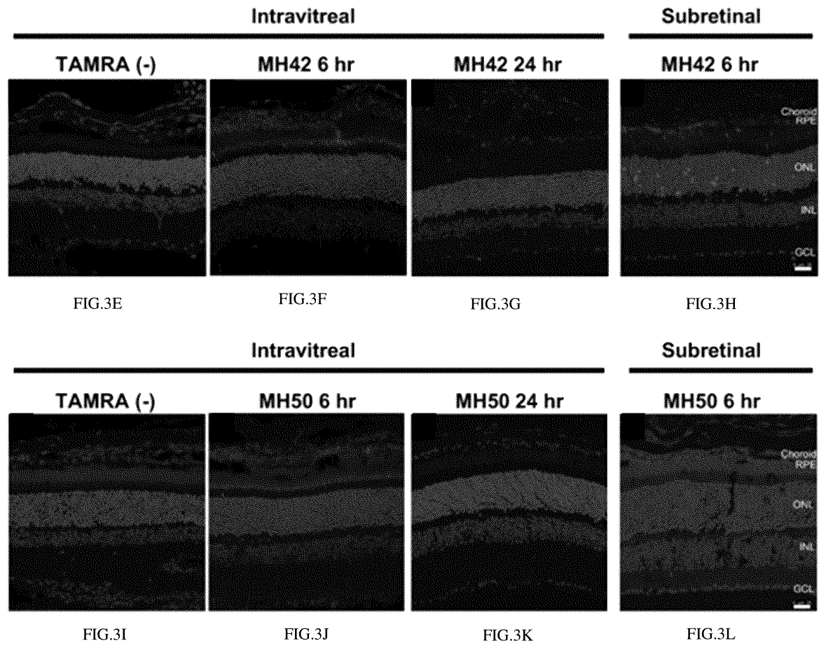

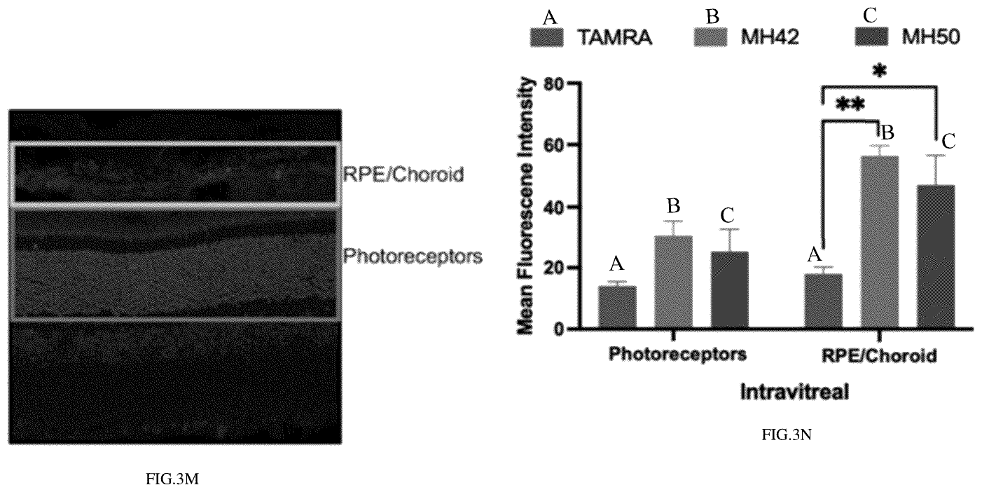

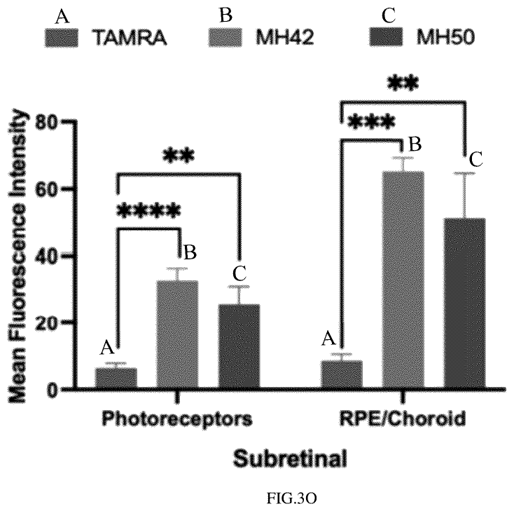

All confocal imaging was performed using TCS SP8 X (Leica Microsystems, Buffalo Grove, IL). Z-stacks (spanned 10 μm with 1 μm interval) were collected using a 20× or 40× objective, and maximum intensity projections were used for further analysis. For the main FIGS. 3A to 3O, mean fluorescent intensity measurements were performed on region-of-interest-gated images for RPE/choroid and PR cell layers specifically using ImageJ.

### NHP In Vivo Delivery and Imaging

One male rhesus macaque, aged 10 years old, was used for this study. All protocols involving NHPs were approved by the ONPRC IACUC and conducted in accordance with the NIH Guidelines for the Care and Use of Laboratory Animals and ARRIVE guidelines. Pupils were dilated to a minimum of 8 mm using phenylephrine (2.5%; Bausch and Lomb, Rochester, NY, USA) and tropicamide (1% Tropicacyl; Akorn, Lake Forest, IL, USA) eye drops. MH42-LNPs were administered into the subretinal space through a 27G/38G subretinal cannula (#5194; Microvision, Redmond, WA, USA) using the Alcon vitrectomy machine and a pars plana trans-vitreal approach. First, a pre-bleb of 30 μl BSS was generated in the posterior pole. Then, 100 μl of MH42-LNP (500 ng/μl) was delivered within and expanded the bleb. After the injection, dexamethasone (0.5 mL, 10 mg/mL) and cefazolin (0.5 mL, 125 mg/mL) were administered subconjunctivally. There were no complications noted during surgery. The animal received comprehensive multimodal retinal imaging before injection (baseline), and at 48 hours post-injection. For each imaging session, the animal was anesthetized by an intramuscular injection of Telazol (1:1 mixture of tiletamine hydrochloride and zolazepam hydrochloride, 3.5-5.0 mg/kg) and maintained with ketamine (1-2 mg/kg) as required. Heart rate and peripheral blood oxygen saturation were monitored by pulse oximetry. Rectal temperature was maintained between 37° C. and 38° C. by water-circulating heated pads. For image acquisition, animals were positioned prone with the head supported by a chinrest; the pupils were dilated to a minimum of 8 mm using phenylephrine (2.5%; Bausch and Lomb, Rochester, NY, USA) and tropicamide (1% Tropicacyl; Akorn, Lake Forest, IL, USA) eye drops. Imaging included wide-field color fundus and autofluorescence (Optos, Inc. Marlborough, MA). Following imaging, the contact lenses and eyelid specula were removed, and erythromycin ointment was applied to each eye.

### NHP Immunohistochemistry and Immunofluorescence Imaging

After the animal was euthanized, eyes were collected and immersion fixed in 4% paraformaldehyde in phosphate buffered saline for 24 to 48 hours. Dissected eyes were then cryoprotected in increasing sucrose gradients (up to 30%), embedded in optimum cutting temperature compound, and frozen in an embedding mold. Frozen blocks were sectioned at 16 μm using a cryostat (CM1850; Leica, Wetzlar, Germany). Sections were collected throughout the bleb. H & E were examined for subretinal bleb injection site retinotomy, transscleral injection site, evidence of immunological reaction, and any signs of pathology. Following examination of the stained sections, adjacent slides were used for immunohistochemistry. LNP expression was identified by colocalization of GFP (anti-GFP, Cat #ab290, Abcam, Cambridge, UK) with glutamine synthetase (Cat #sc74430, Santa Cruz Biotechnologies, CA), rod and cone arrestin (generously provided by W. Clay Smith, PhD, University of Florida), and RPE65 (Cat #ab13826, Abcam). Immune infiltrates were identified using antibodies against IBA-1 (microglia; Cat #019-19741 Wako Chemicals, Richmond, VA) and CD3 (T-cells; Cat #sc20047, Santa Cruz Biotechnologies), respectively. All primary antibodies were used at a concentration of 1:500, except for anti-CD3 and anti-rod arrestin which were used at 1:50. Detection of primary antibodies was achieved using Alexafluor secondary antibodies at a concentration of 1:300; 4′,6-diamidino-2-phenylindole (DAPI) was used as a nuclear marker counterstain.

### Statistical Analysis

For TAMRA quantification from peptide injections, region of interest (ROI) were created for RPE/choroid and PR cell layer for mean fluorescent intensity values. Fold change measurements were compared to TAMRA-only controls. An ordinary one-way ANOVA, with Tukey's correction for multiple comparisons test was used for comparisons between groups (Prism 8 software, GraphPad Software, La Jolla, CA). Data are presented as mean±SEM. A p-value <0.05 was considered as statistically significant.

### Cryo-EM

Vitrification of samples was performed by dispensing 2 μL of LNPs onto a glow-discharged 300 mesh Lacey Carbon film-coated copper grid using Vitribot Mark IV (FEI). Grids were blotted with filter paper for 3 seconds at 22° C. and 100% relative humidity thereafter grids were plunged into the copper cup containing liquid ethane cooled by liquid nitrogen. Any defect in frozen grids was carefully checked, clipped, and assembled into cassettes. After vitrification, frozen grids were maintained at a temperature below −170° C. using liquid nitrogen. Imaging was performed in 300 keV Glacios Cryo-EM equipped with Falcon III and K3 Summit camera (Gatan) with DED in counting mode with a magnification of 45,000× at an electron dose of ˜15-20 e−/Å2. The captured images were then processed and analyzed manually using ImageJ software.

### RESULTS

### In Vivo Bacteriophage Biopanning

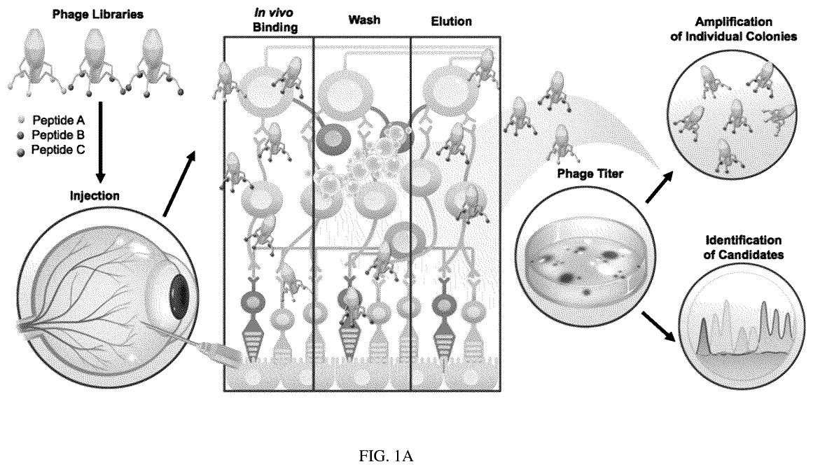

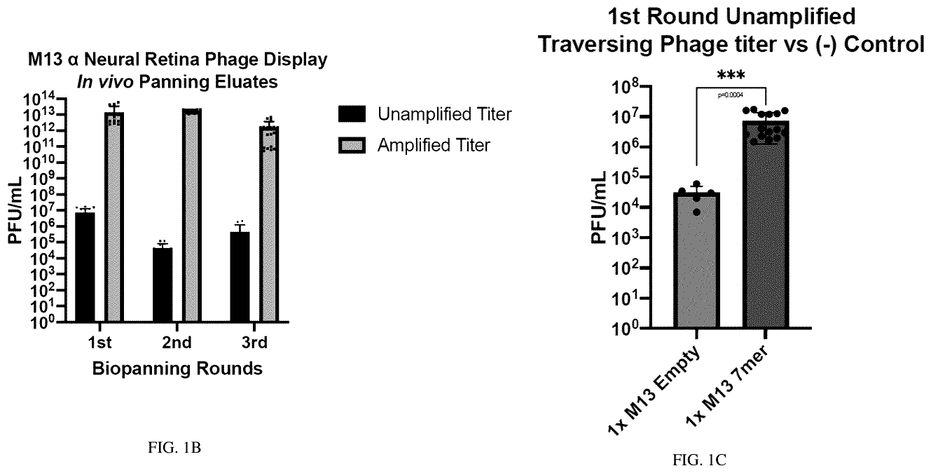

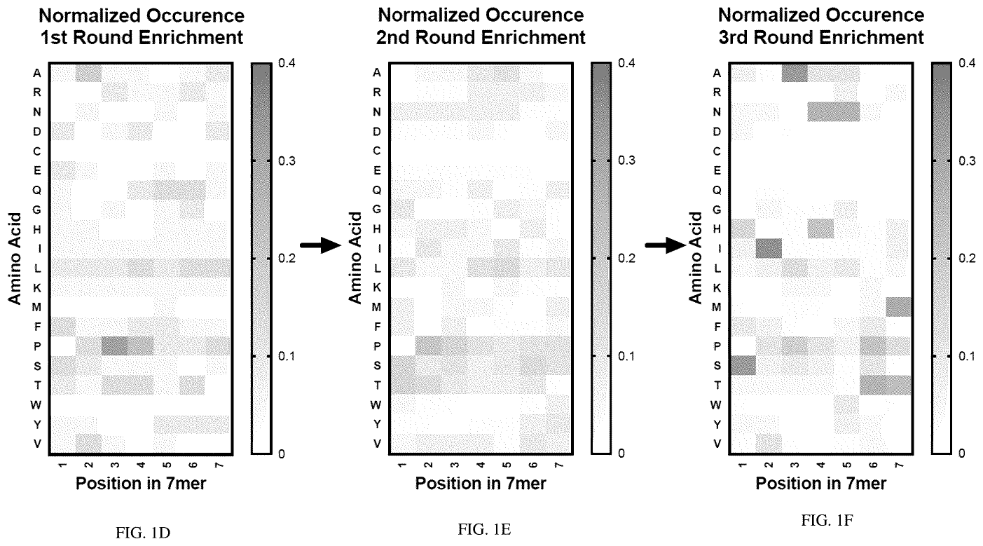

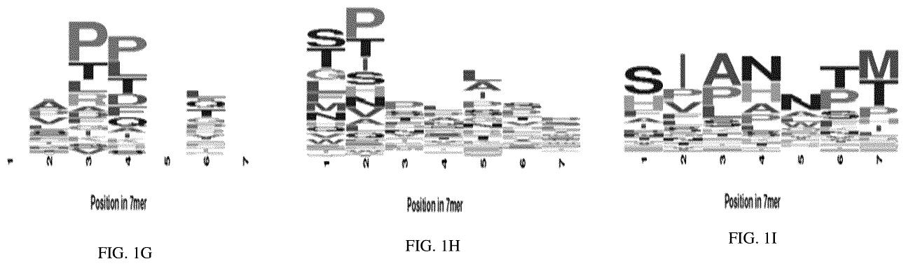

In order to elucidate retina-specific peptide sequences, a naïve heptameric M13 bacteriophage library was injected intravitreally in mice. At 6 hours post-injection, the neural retina was harvested, washed, and bound bacteriophage-peptide fusions were eluted off the tissue. Collected bacteriophage-peptide fusions were either amplified for another round of injections (enriched library) or sequenced to elucidate the specific bacteriophage-peptide fusions that bound to the neural retina after intravitreal delivery (FIG. 1A). The initial validation experiment using the naïve M13 bacteriophage library against streptavidin showed that three iterative rounds of biopanning were necessary to isolate a known balbsequence (Table 2). Thus, when using the M13 bacteriophage library in vivo, BALB/c mice were injected intravitreally for three iterative rounds of biopanning.

TABLE 1

| 1^{st} Round | 2^{nd} Round | 3^{rd} Round |
|---|---|---|
| YHQTVQC (SEQ ID No. 38) | RLGLEGP (SEQ ID No. 50) | NLLSHPQ (SEQ ID No. 51) |
| QLSTADV (SEQ ID No. 39) | NLLSHPQ (SEQ ID No. 51) | NLLSHPQ (SEQ ID No. 51) |
| NPRFYPS (SEQ ID No. 40) | NLLSHPQ (SEQ ID No. 51) | NLLSHPQ (SEQ ID No. 51) |
| ESCHQFP (SEQ ID No. 41) | NLLSHPQ (SEQ ID No. 51) | NLLSHPQ (SEQ ID No. 51) |
| QTPATAG (SEQ ID No. 42) | NLLSHPQ (SEQ ID No. 51) | NLLSHPQ (SEQ ID No. 51) |
| TSPSYSM (SEQ ID No. 43) | NLLSHPQ (SEQ ID No. 51) | NLLSHPQ (SEQ ID No. 51) |
| YSRDSAA (SEQ ID No. 44) | NLLSHPQ (SEQ ID No. 51) | NLLSHPQ (SEQ ID No. 51) |
| HMTVSVF (SEQ ID No. 45) | NLLSHPQ (SEQ ID No. 51) | NLLSHPQ (SEQ ID No. 51) |
| CPRMPSV (SEQ ID No. 46) | NLLSHPQ (SEQ ID No. 51) | NLLSHPQ (SEQ ID No. 51) |
| YHNMLPP (SEQ ID No. 47) | NLLSHPQ (SEQ ID No. 51) | NLLSHPQ (SEQ ID No. 51) |
| SYAPHIA (SEQ ID No. 48) | NLLSHPQ (SEQ ID No. 51) | NLLSHPQ (SEQ ID No. 51) |
| TVRYVEP (SEQ ID No. 49) | NLLSHPQ (SEQ ID No. 51) | NLLSHPQ (SEQ ID No. 51) |
| | NLLSHPQ (SEQ ID No. 51) | NLLSHPQ (SEQ ID No. 51) |
| | NLLSHPQ (SEQ ID No. 51) | NLLSHPQ (SEQ ID No. 51) |
| | NLLSHPQ (SEQ ID No. 51) | NLLSHPQ (SEQ ID No. 51) |

Titers were measured after each round, quantifying the amount of unamplified bacteriophage-peptide fusions isolated from the tissue, and then amplified in culture (enriched library) for subsequent panning round injection. Titers measured after each round of in vivo biopanning were detected within the expected ranges (FIG. 1B). An empty, M13 bacteriophage library, delivered at an equivalent concentration, was used as a negative controls. Titers retrieved from the neural retina after the first round of biopanning were higher when using the full M13 bacteriophage library compared to the empty M13 bacteriophage library (FIG. 1C). A 100-fold higher bacteriophage-peptide count from the naïve library biopanning suggests that the displayed peptides were facilitating the binding of the neutral retina, as opposed to other proteins on the surface of the empty bacteriophage itself (FIG. 1C). After each round of biopanning, eluted unamplified phage plaques were Sanger sequenced and the normalized occurrence of each amino acid at each position in the heptameric moiety was displayed to elucidate the enrichment across the three rounds. After the third round of biopanning, SIA(N/H)NT(M/T) (SEQ ID No. 37) appeared as a motif, which is shown in the heat maps and accompanying enrichment plots (FIGS. 1D to 1I). Overall, after the third round of biopanning, over 150 phage plaques were sequenced and 30 unique sequences were identified for retinal targeting showcasing the attained enrichment (Table 2).

### Differential Binding of In Vivo Isolated Phage-Displayed Peptides

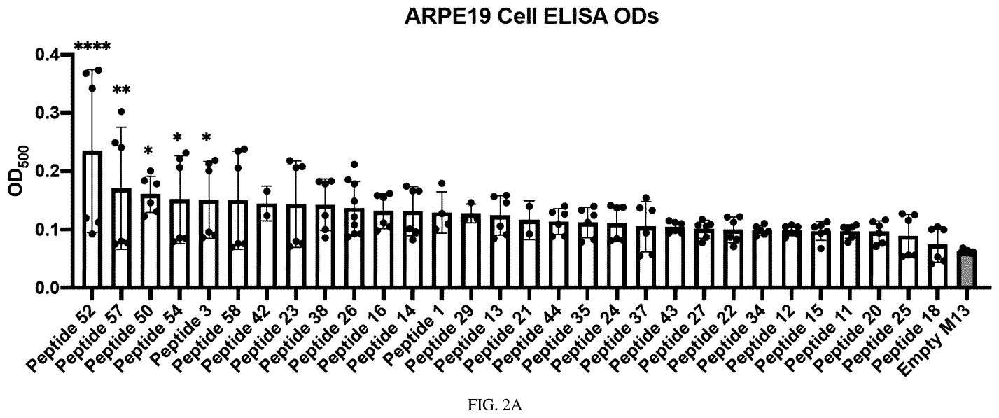

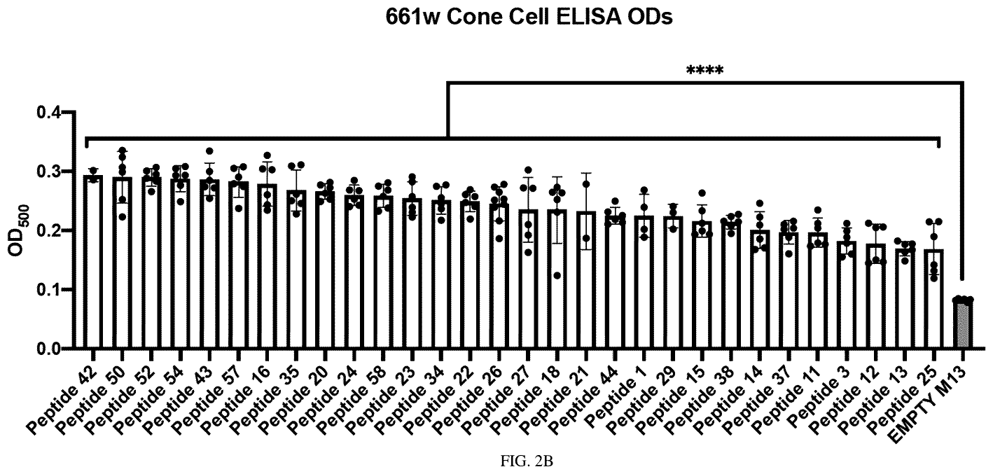

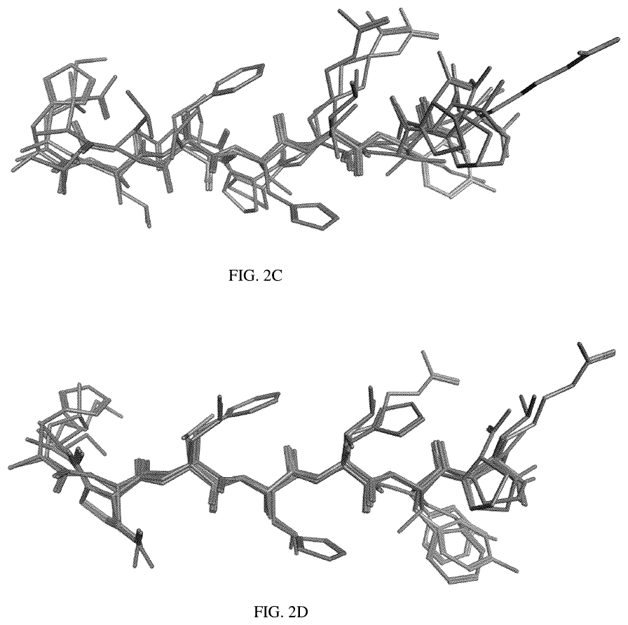

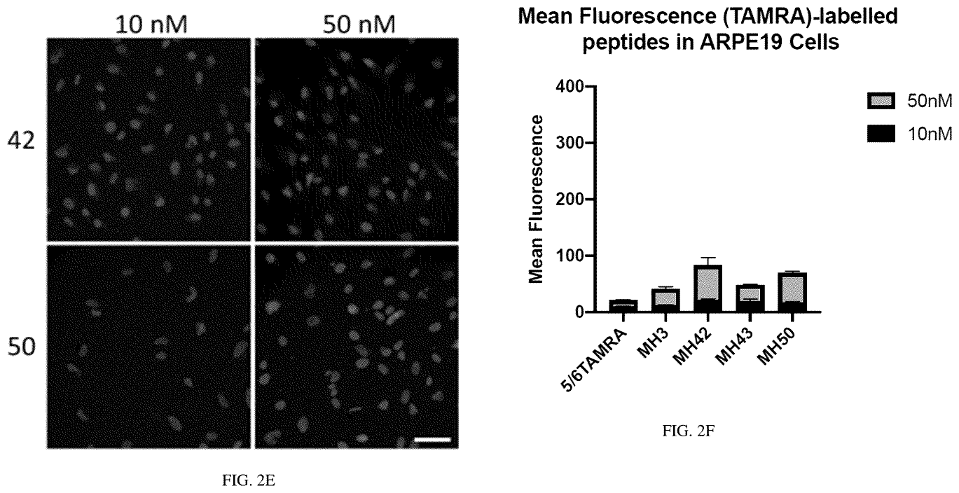

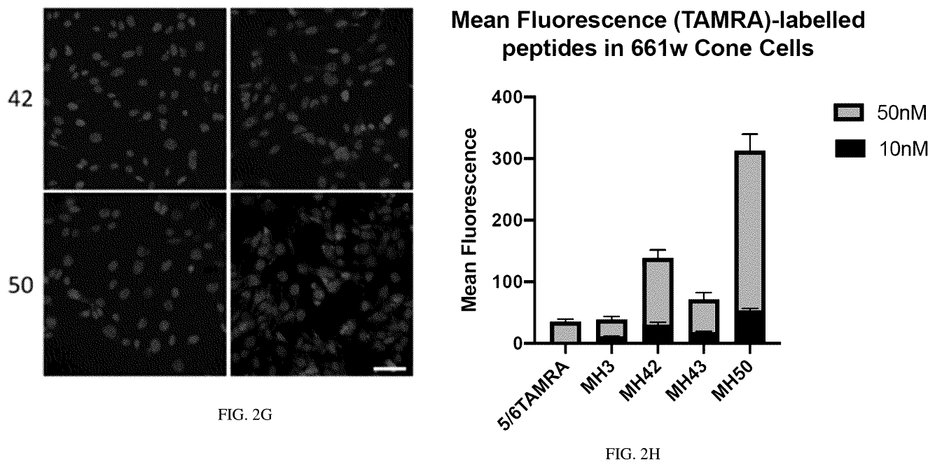

After identifying 30 novel peptide sequences, top-performing candidates were identified that could target LNPs to the neural retina. Since bacteriophages were eluted from the entire neural retina, the candidates were pre-screened for their ability to bind to the cell of interest, the PRs. Single bacteriophage-peptides were amplified in E. coli, and a cell-based enzyme-linked immunosorbent assay (ELISA) was developed against the 661w mouse cone PR cell line and the hARPE19 cell line as a control. As expected, for most of the bacteriophage-peptide candidates, binding affinities were weaker when tested against RPE cells compared to 661w cone cells. However, in hARPE19 cells, peptides MH52, MH57, MH50, MH42, MH54 and MH3 showed significantly higher binding to RPE cells compared to control (FIG. 2A, *p≤0.05, ****p≤0.0001). In the 661w cone cells, all 30 phage-peptides tested exhibited significantly increased binding affinities compared to empty bacteriophage control. highlighting their neural retina affinity (FIG. 2B, ****p≤0.0001). Some phage-peptide fusions exhibited high binding across both of the ocular cell lines, such as peptides MH42, MH50, MH52 and MH57. However, the binding affinity, as measured by OD450, was close to 2-fold higher when binding to 661w cone cells compared to hARPE19 cells. After confirming binding affinity to the cell of interest, analysis continued with 5 peptides that had the highest affinity for 661w cells (peptides 42, 43, 50, 52, 54) and ARPE19 cells (peptides 3, 50, 52, 54, 57) as a control (Tables 2, 3, 4, and 5).

### Pharmaceutically Relevant Properties and 3D Superposition of Peptide Sequences Tested

The physicochemical properties of the peptide sequences as well as their structure governed by amino acid composition was analyzed using the QikProp module of Schrodinger drug discovery suite and Molecular Operating Environment software. Pharmaceutically-relevant physicochemical properties were computed and reported for all of the candidate peptide sequences tested (Table 2), while those that had highest binding against 661w cone cells and hARPE19 cells are highlighted (Tables 2 and 4). As expected, for hARPE19 top performers, there were no conserved structural patterns of pertinence in the structural superposition analysis (FIG. 2C). For 661w cone cell top candidates, the structural superposition of physiologically relevant conformations showed a high conservation in 3D models across all five peptides (FIG. 2D). In general, the top peptides were made up of mostly neutral, slightly basic encouraging amino acid residues, which was preferable given their compatibility with our LNP carriers. and required endosomal escape-prone profile (Table 3 and 2). In contrast, MH3, which showed reduced binding affinity in the 661w cone cells, contained slightly more acidic amino acid residues (Table 5).

### Cell Internalization of Fluorescent-Labeled Peptides

Although differential binding results were promising, prior to LNP conjugation, cellular internalization of peptide ligands was confirmed. Four peptides were chosen from the 661w cone cell-ELISA results; three high-binders (MH50, MH43, MH42) and one low-binder (MH3) to serve as negative control (FIGS. 2A to 2B). These peptides were synthesized with a carboxy-end linker, -GGGS (Table 2), followed by a carboxy-terminal cysteine that was conjugated to a 5-(and −6)-Carboxytetramethylrhodamine (TAMRA) dye.

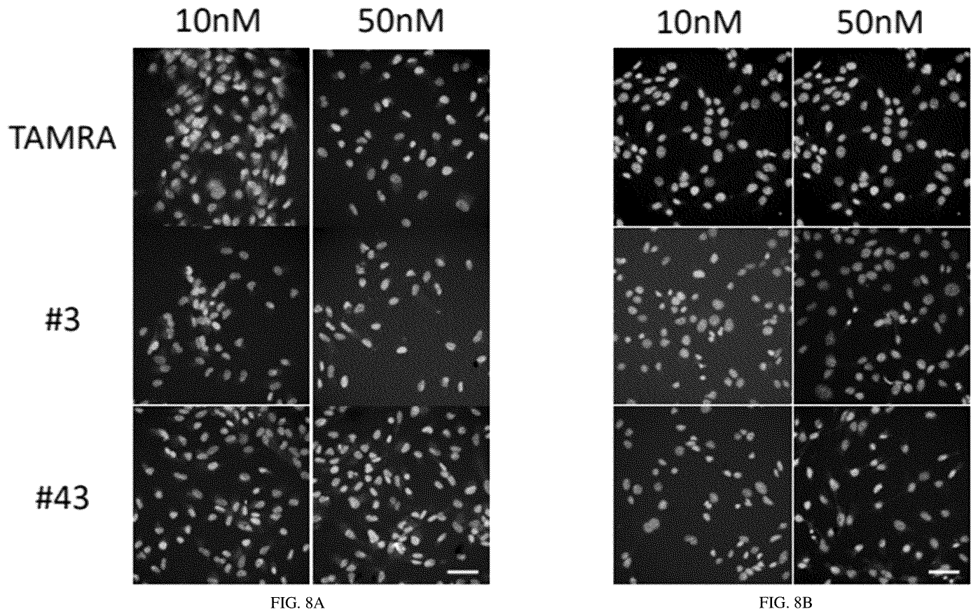

TAMRA-conjugated peptides were added to hARPE19 and 661w cells at a 10 nM and 50 nM concentration to evaluate their internalization (FIGS. 2E to 2H and 8A to 8B). After 30 minutes of incubation at 37° C., cells were fixed and DAPI stained for confocal imaging. 2.3× higher uptake was observed in 661w cone cells when compared to RPE cells as measured by mean fluorescence intensity across peptides tested at concentrations of 10 nM and 50 nM (FIGS. 2F to 2H). Peptides MH50 and MH42 elicited 4.1- and 1.7-fold higher cellular uptake compared to the negative control MH3 in 661w cells respectively. MH42 and MH50 were also the top 2 performing peptides in the 661w differential binding affinity ELISA. Taken together, these in vitro validation studies suggest that these peptides can bind to and be internalized into our cells of interest (FIG. 2).

In Vivo Validation of Labelled Peptides with Conjugated TAMRA Dye

After verifying binding and internalization in vitro, penetrative properties and targeting capabilities of the TAMRA-labelled peptide candidates were tested in BALB/c mice. First, the pharmacokinetic profile of the peptides candidates was determined. All peptides conjugated to TAMRA dye were injected intravitreally and eyes were harvested at 1 hour, 6 hours and 24 hours post-administration (FIGS. 3 & 9). TAMRA dye was visible in the retina at 1 hour post-injection, however, maximum levels of labelled peptide were observed at the 6 hour timepoint. Peptides were cleared by 24 hours post-injection (FIGS. 3 & 9). In vivo fundus imaging post MH42 injection supported these findings and showed the highest TAMRA fluorescence intensity at 6 hours and whole clearance by 24 hours (FIGS. 3A to 3D). With maximum localization of labelled peptide observed at 6 hours, all quantification was performed at the 6 hour timepoint. In addition to intravitreal delivery, TAMRA-labelled peptides were also injected subretinally and harvested at the 6 hour time point (FIGS. 3 & 9).

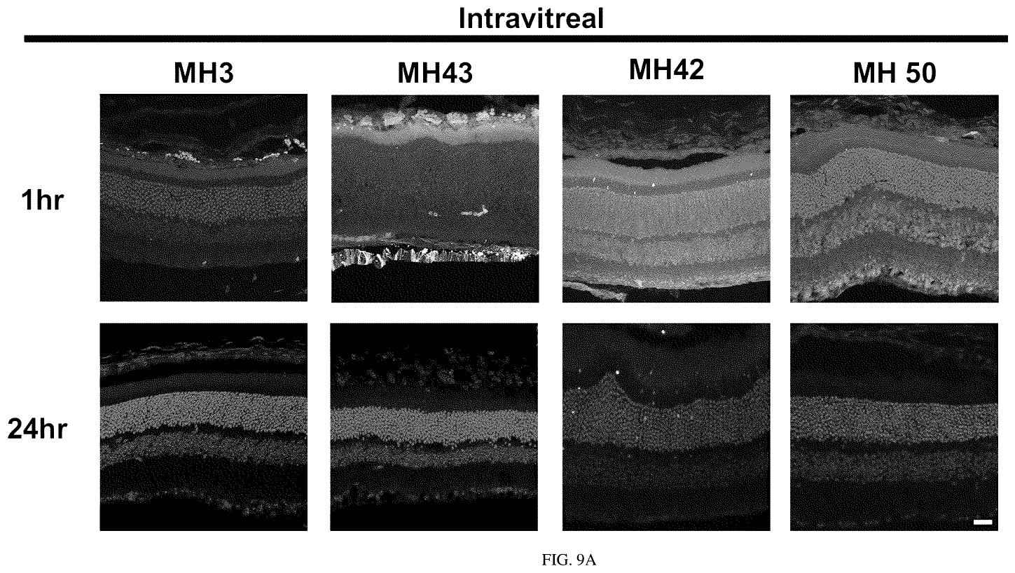

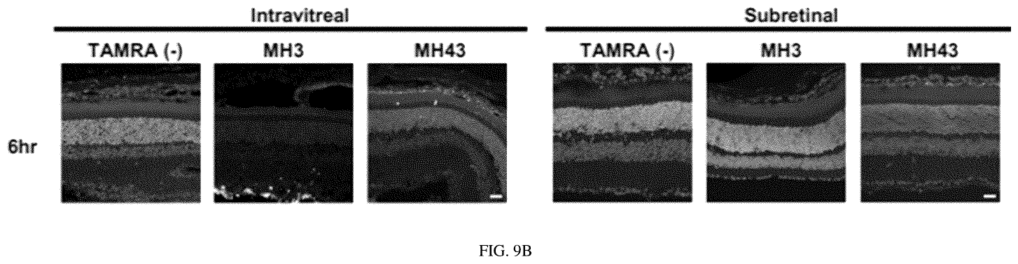

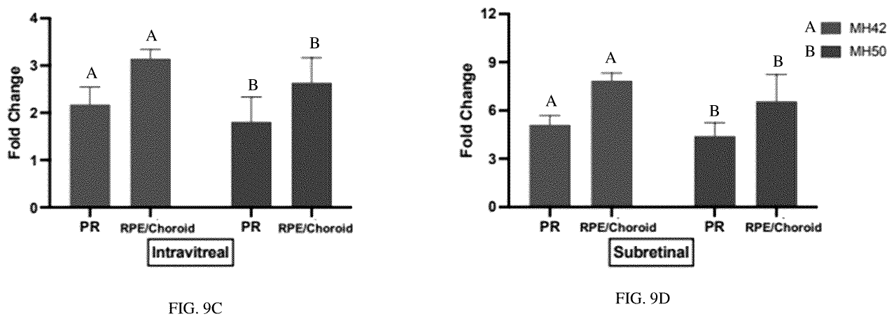

Both MH42 and MH50 were localized to PRs including the outer nuclear layer and inner/outer segments as well as RPE (FIGS. 3E to 3L). For quantification, the PR and RPE/choroid layers were segmented to obtain mean fluorescent intensity (FIG. 3M). When injected intravitreally, MH42 demonstrated 2.2- and 3.2-fold increase in PR and RPE/choroid localization compared to TAMRA-dye injection control, respectively (FIGS. 3N & 9C, **p≤0.01). With MH42 subretinal administration, increased PR and RPE/choroid localization was observed by 5.1- and 7.9-fold respectively (FIGS. 3O and 9D **p≤0.01, ***p≤0.001, ****p≤0.0001). For MH50, PR and RPE/choroid localization was increased by 1.8- and 2.6-fold after intravitreal administration and by 4.4- and 6.6-fold after subretinal administration (FIGS. 3N to 3O and 9C to 9D, *p≤0.05, **p≤0.01). As expected, the negative control, MH3, showed poor accumulation in RPE and PRs at all timepoints tested post-injection. In the case of peptide MH43, affinity towards the PRs was observed at the 6 hour timepoint post-intravitreal injection, but not post-subretinal injection (FIGS. 9A to 9B).

In Vivo Validation of Targeting Peptides on Surface of Cre mRNA-Loaded Lipid Nanoparticles in Ai9 mice

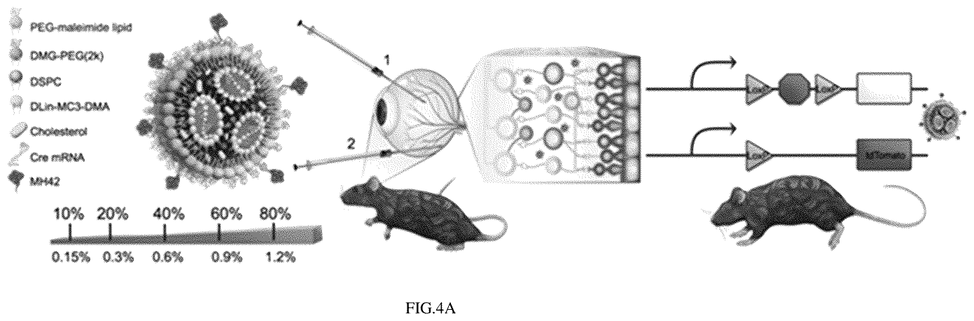

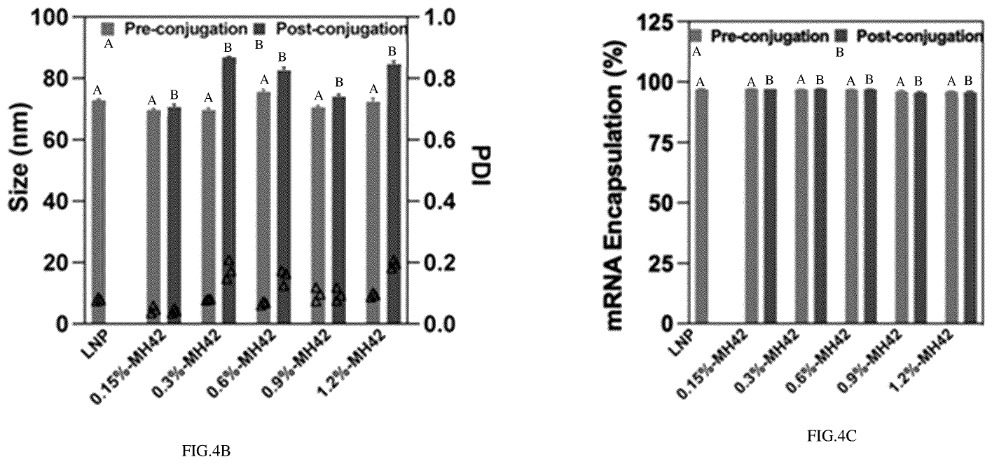

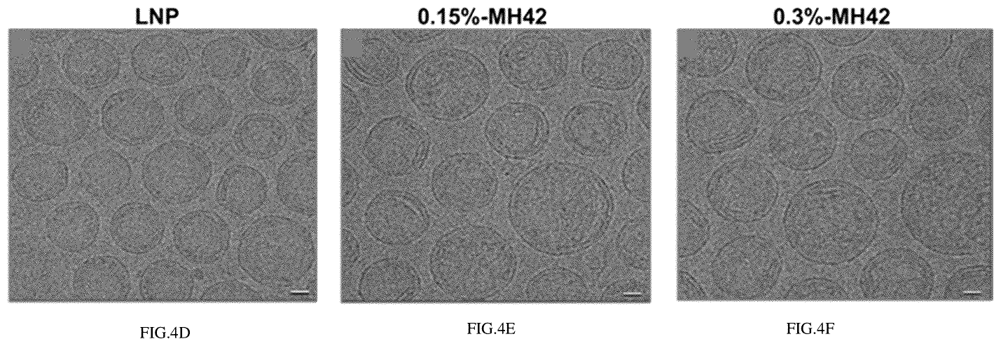

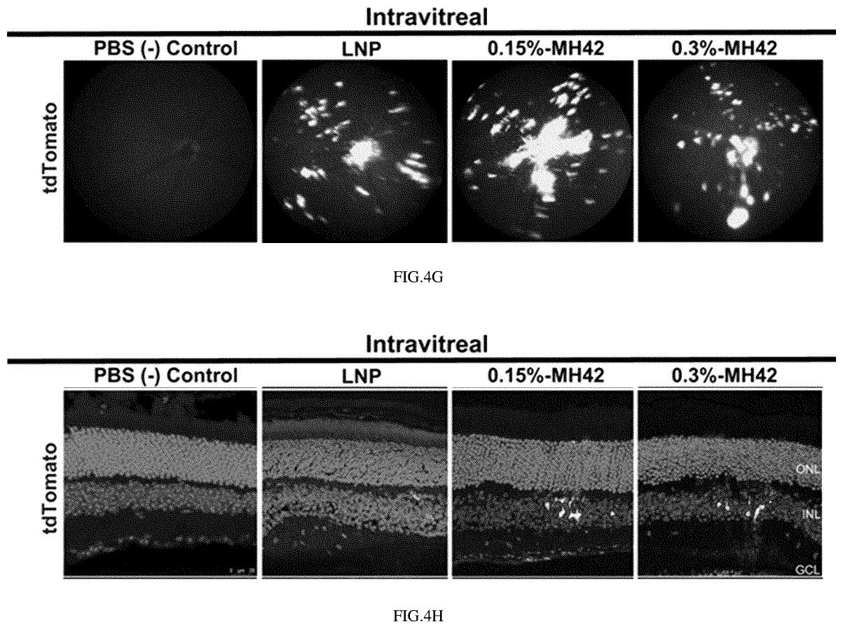

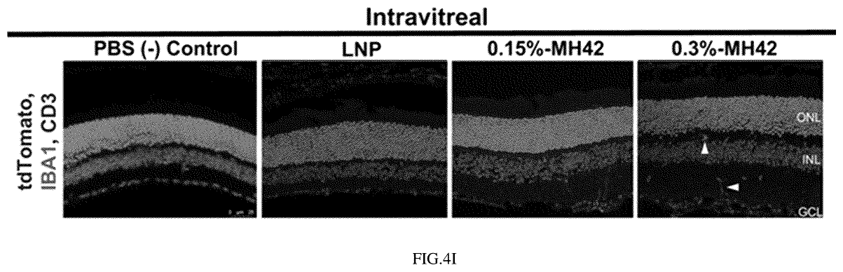

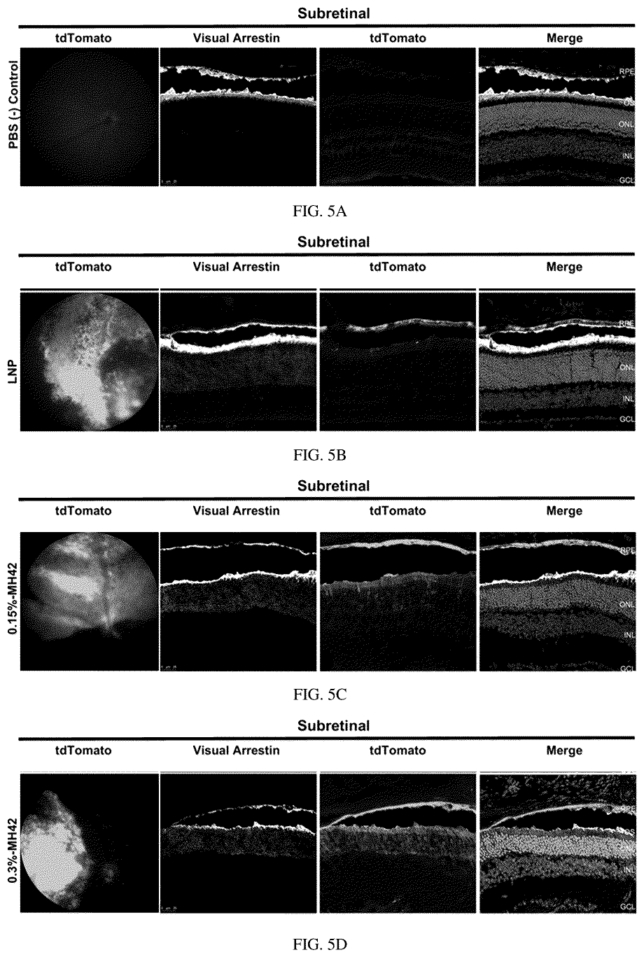

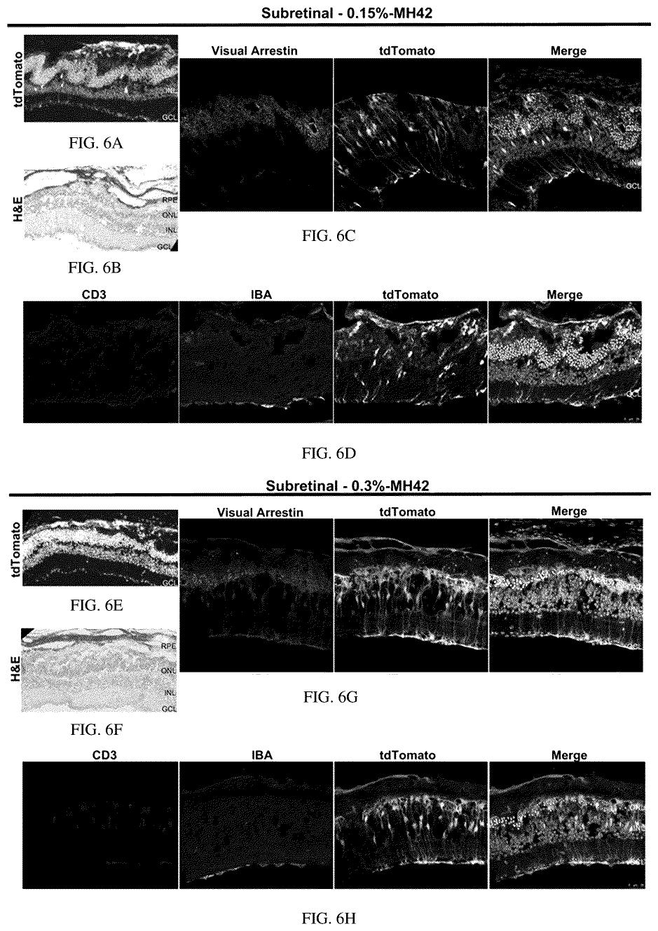

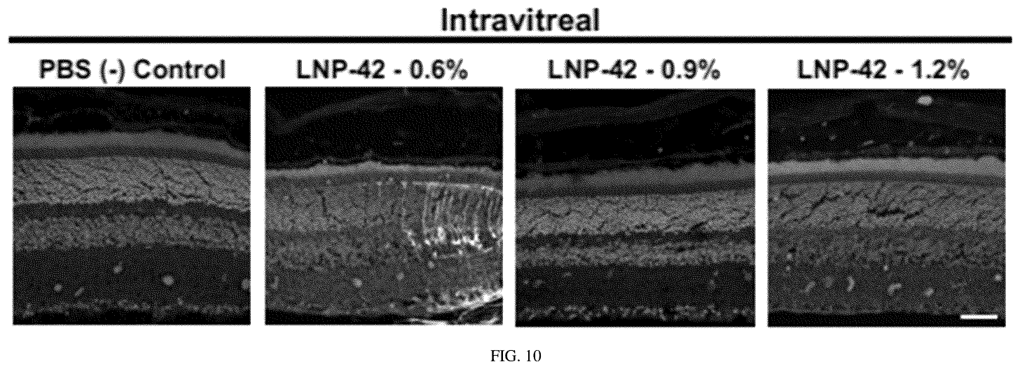

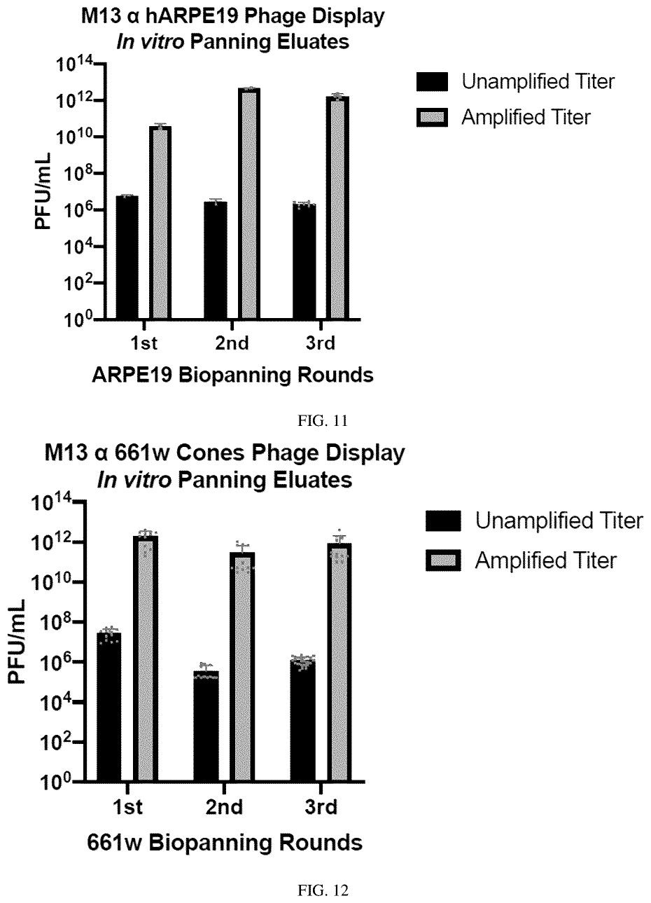

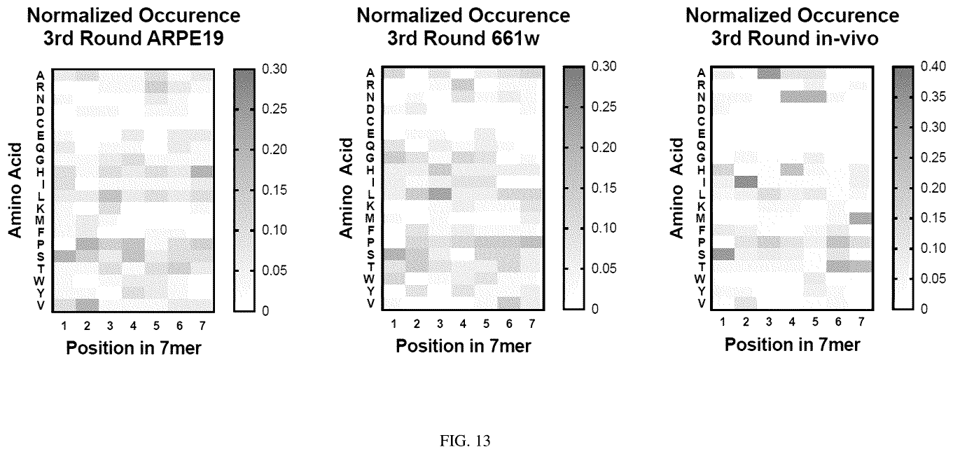

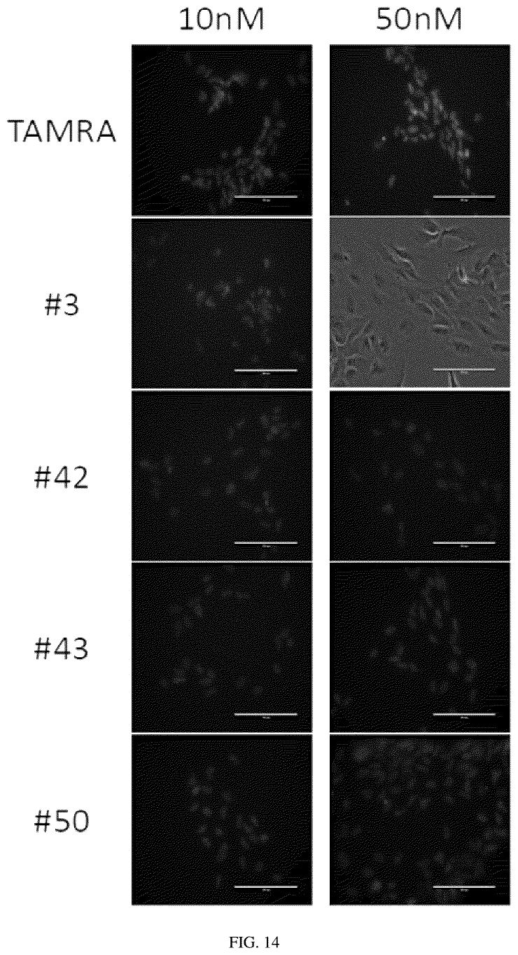

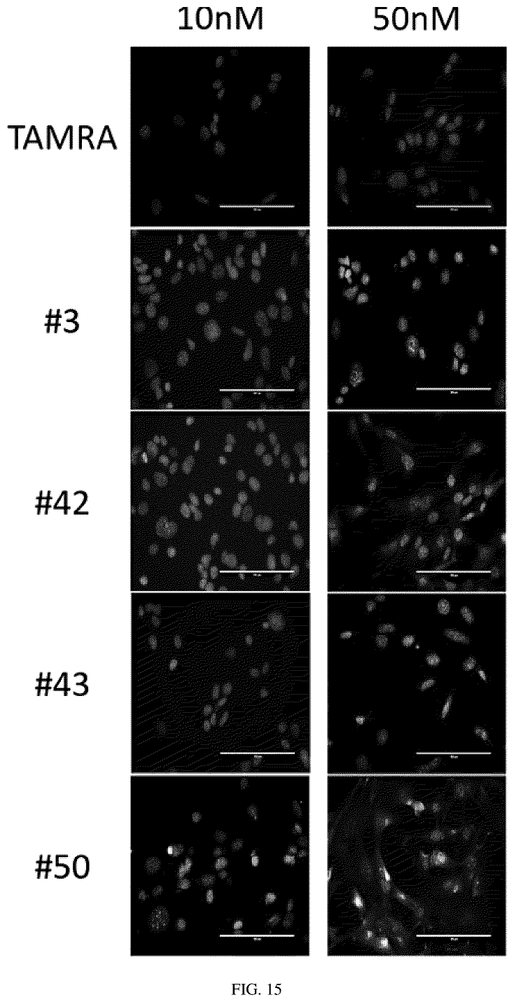

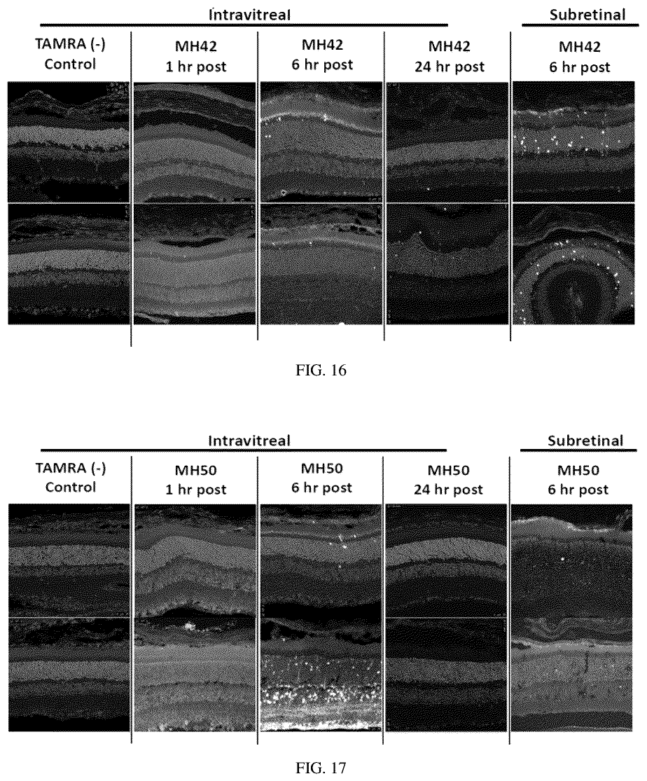

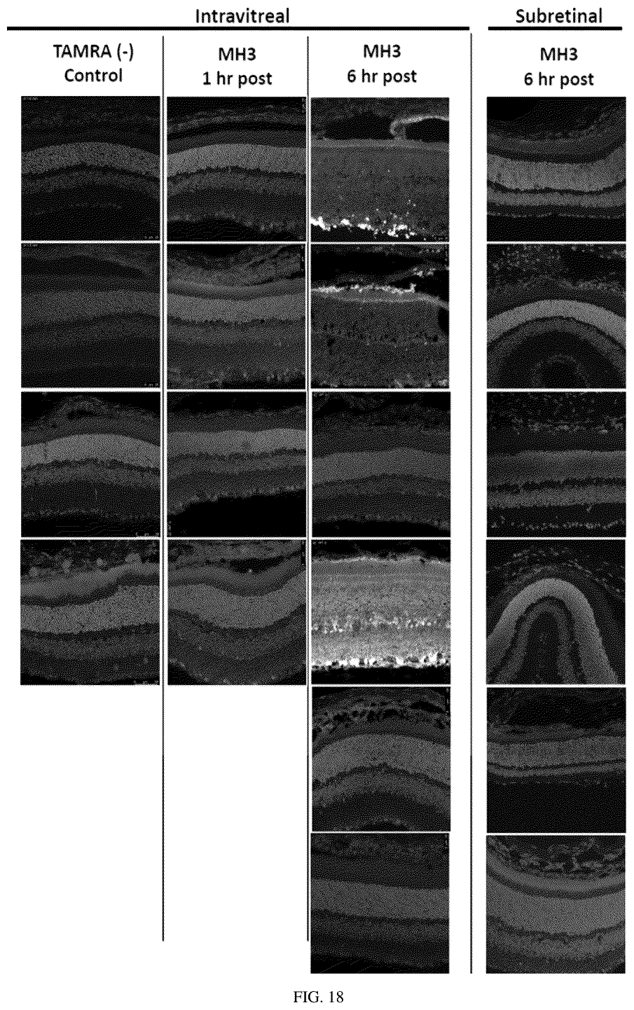

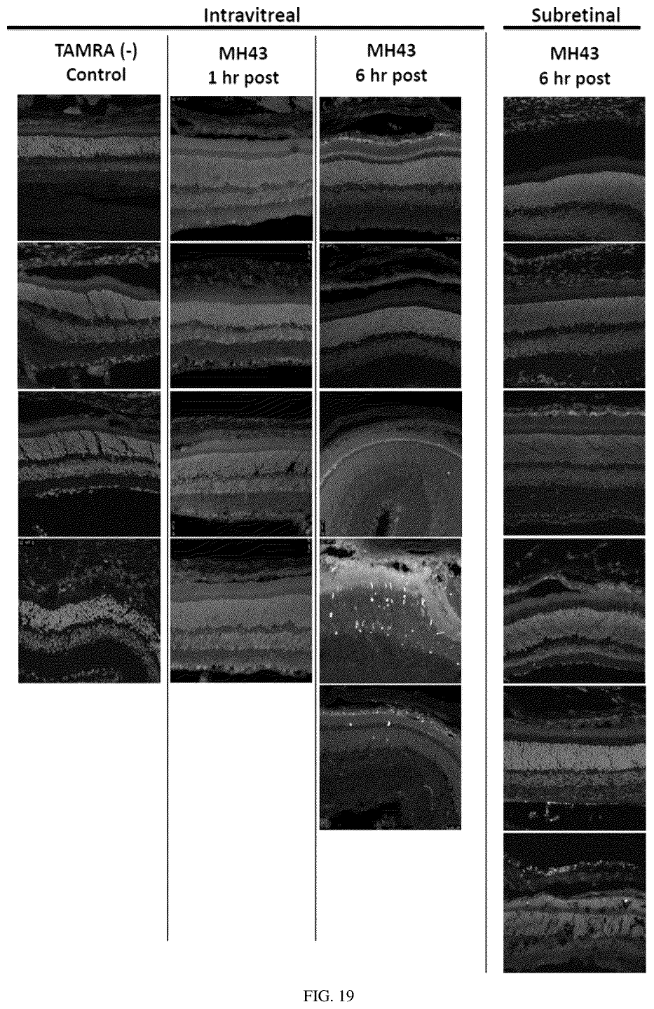

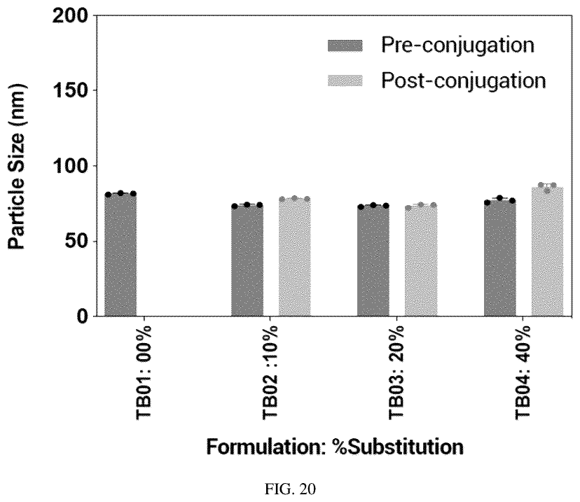

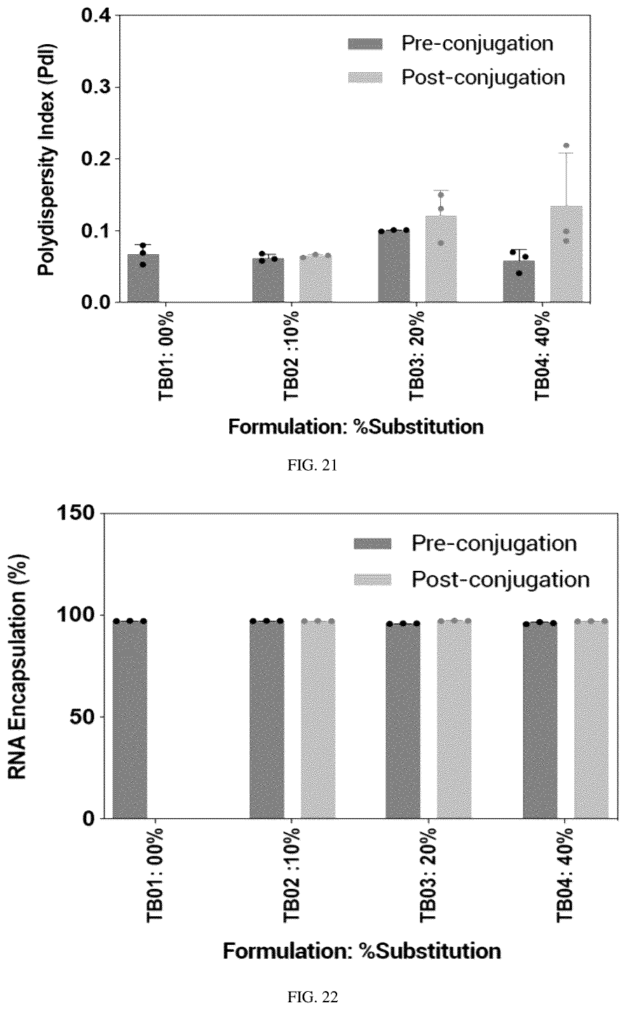

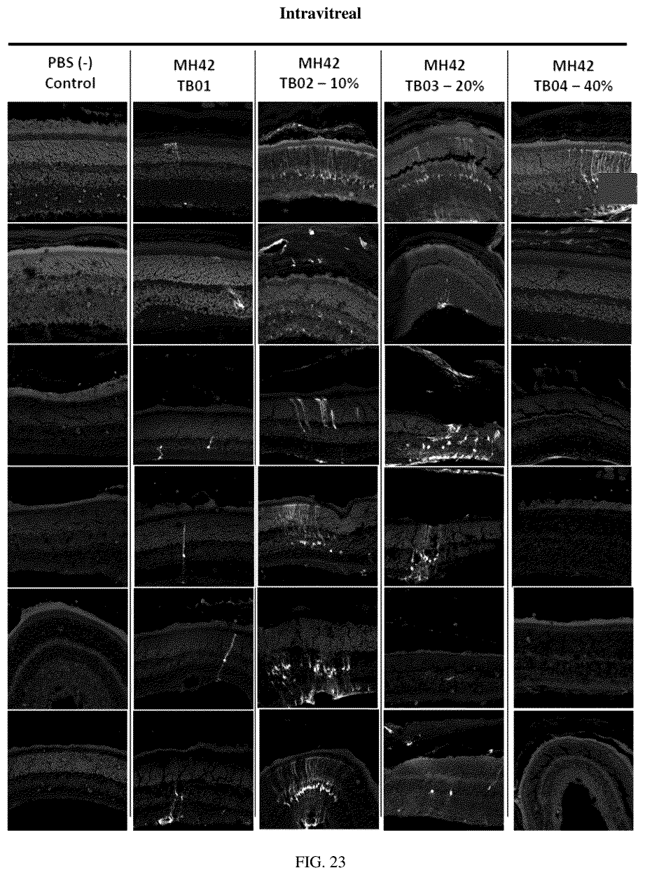

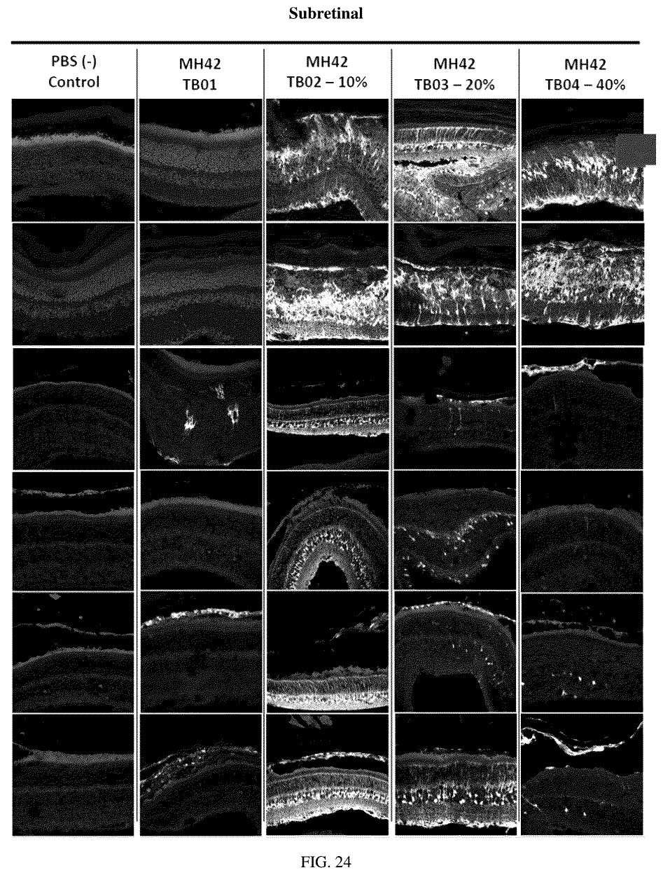

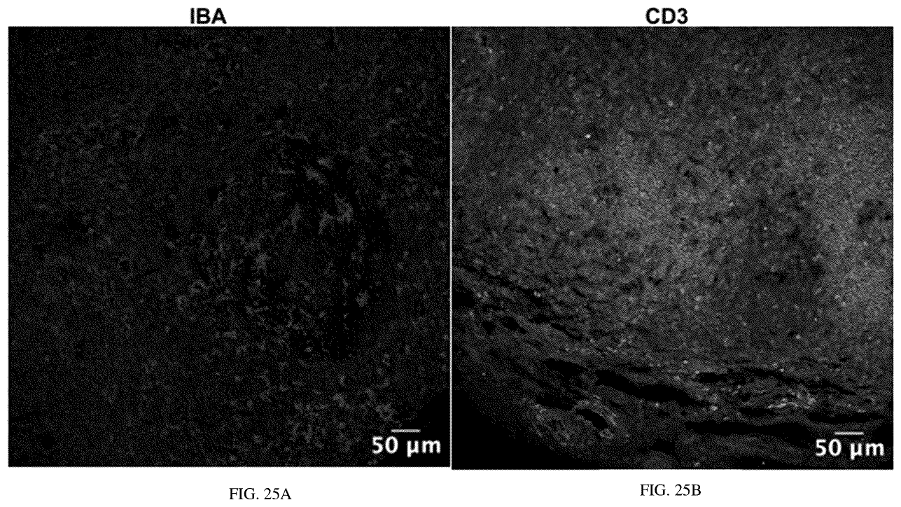

Encouraged with the localization of TAMRA-dye conjugated peptide candidates in BALB/c mice, a gene delivery reporter mouse model was used to visualize cell-based gene editing. Ai-9 mice stably express a floxed stop codon upstream of a tdTomato cassette in all cells which is only translated after successful Cre-recombinase delivery recombination (FIG. 4A). Cre-mRNA was encapsulated inside of LNPs formulated with DLin-MC3-DMA, DSPC, cholesterol, and DMG-PEG2K as the ionizable, ionizable, structural, and PEG-lipid, respectively (FIG. 4A). For LNP-peptide conjugates, molar equivalents of the PEG-lipid component were substituted for varying amounts of functionalized PEG lipid, in this case DSPE-PEG2k-Maleimide, for direct conjugation to the peptides via substituted carboxy-end cysteine. The carboxy-end of the peptide ligand's terminal cysteine was amide-capped to mimic the physicochemical properties of the original peptide obtained from the screenings. The free —SH group on the cysteine side chain facilitated direction conjugation onto the surface of our LNP with the PEG lipid via thio-ester conjugation. A wide range of targeting ligand surface densities were explored, evaluating a gradient from 0.15% to 1.2% of the 1.5% total PEG-lipid content, equivalent to 10%-80% of total PEG (Table 10). LNPs with varying levels of ligand on surface were formulated with Cre-recombinase mRNA, and subsequently injected into Ai-9 mice. Unconjugated LNPs, loaded with Cre-mRNA served as non-targeted baseline comparison in the studies. LNP characterization using dynamic light scattering (DLS) as well as nanoparticle tracking analysis (NTA) measured LNP size as 70-76 nm in diameter before peptide conjugation with a uniform polydispersity index (PDI) of 0.1 indicating a homogeneous preparation. Post conjugation, LNP-MH42 conjugates displayed slightly higher diameters (71-87 nm) as well as an increased PDI likely due to the inclusion of peptides on their surface (FIG. 4B). mRNA encapsulation ranged between 95% and 99% for all LNPs ensuring almost complete encapsulation of cargo across all formulations tested irrespective of ligand conjugation amount (FIG. 4C). Cryo-EM of LNPs with and without peptides corroborated once again that LNP morphology was undisturbed by the conjugation strategy (FIGS. 4D to 4F). To quantify the amount of peptide conjugated on surface of nanoparticle, a fluorometric absorbance kit that detects free maleimide groups was utilized before and after conjugation. The calculated total amount (w/v) of peptide was 376 μg and 492 μg for 0.15% and 0.3%-MH42 per p L LNP solution, respectively. At 7 days post-intravitreal or -subretinal administration of LNPs with and without MH42, tdTomato expression was visualized in vivo with fundus imaging and post-mortem with confocal microscopy of retinal cryosections. Initially, untargeted LNP were tested, as well as five different conjugated preparations, spanning 10-80% of total 1.5% PEG content, intravitreally (FIGS. 5G to 5I and FIG. 10). MH42 at 0.15% (10% of total PEG), 0.3% (20% of total PEG) and 0.6% (40% of total PEG) showed Müller glia transfection after intravitreal administration (FIGS. 5G to 5H and 10). Müller glia transfection was not associated with any signs of an immune response or retinal toxicity. There was no T cell infiltration as evident by CD3 staining and the microglia, labeled with IBA-1, were restricted to the plexiform layers (FIG. 4I, arrows). Positive controls for IBA-1 and CD3 stains are as described herein (e.g., in FIGS. 25A to 25B). After subretinal administration of untargeted LNPs, transfection was predominately observed in the RPE, as previously published (24, 25) (FIG. 5B). However, with the addition of MH42 at 0.15% and 0.3%, tdTomato expression was observed in the RPE and PRs. Retinal cross-sections were co-stained with visual arrestin, which labels the cytoplasm of rod and cone cell bodies as well as the entire outer segments (FIGS. 5A to 5D). Visual arrestin labeling aligned with the tdTomato expression, confirming PR delivery by MH42-LNP (FIGS. 5C to 5D). Areas of MH42-LNP injected retinas contained disrupted retinal morphology corresponding with the loss of the PRs (FIGS. 6A to 6B and 6E to 6F). More specifically, there was a loss of PR inner/outer segments and the formation of outer nuclear layer rosettes (FIGS. 6C and 6G). There was still presence of PR transfection, but more strikingly, there was an increase in Müller glia transfection in these areas (FIGS. 6C and 6G). Retinal cross-sections were stained with IBA-1 and CD3 in order to detect an immune response corresponding to the retinal damage (FIGS. 6D and 6H). Interestingly, T cell infiltration was not detected and there was no evidence of microglia activation (FIGS. 6D and 6H). Even with these retinal disruptions, overall, it was determined that 0.15 and 0.3% peptide substitution were useful for mRNA delivery of ligand-functionalized LNPs. (FIGS. 4A to 6H).

### Subretinal Delivery to NHP Retina

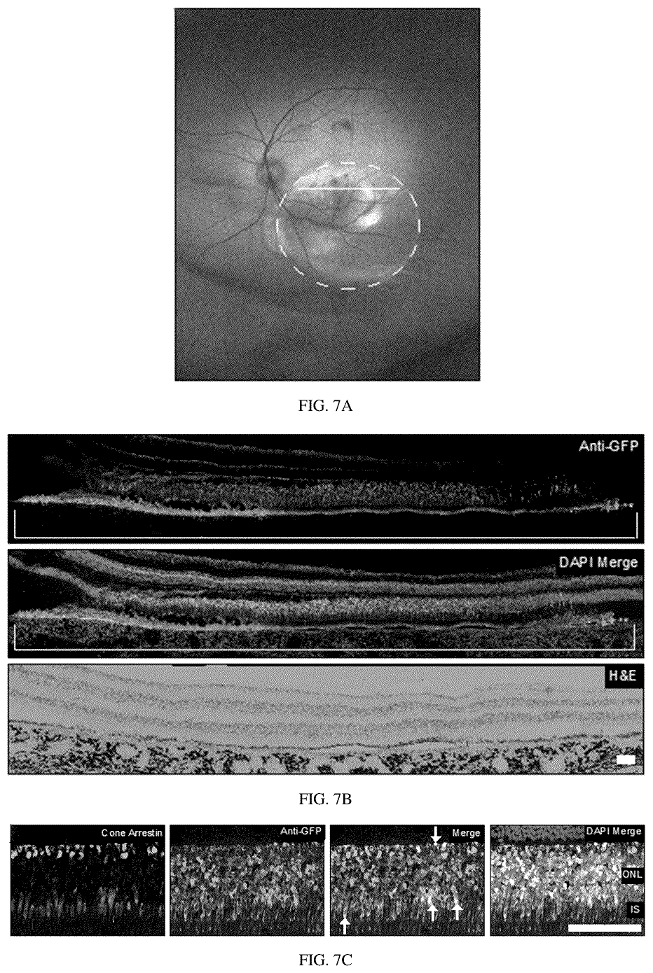

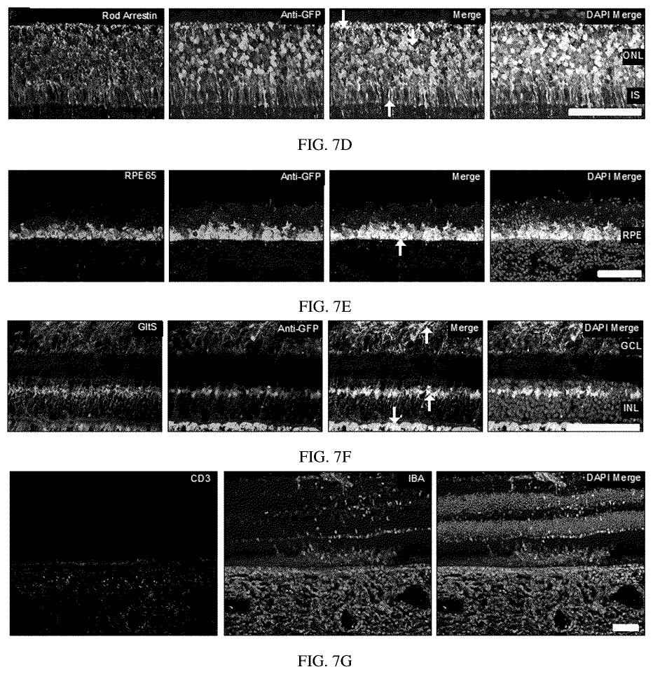

Since subretinal delivery with the disclosed formulations showed PR transfection, the translatability of 0.15%-MH42 LNP mediated mRNA delivery was explored in a highly relevant NHP model, the rhesus macaque. GFP mRNA LNPs were formulated using cholesterol, DLin-MC3-DMA, DSPC, and DMG-PEG2K. For direct conjugation to the peptides, 0.15% molar equivalent of the PEG-lipid component was substituted for functionalized PEG, DSPE-PEG2k-Carboxy-NHS. MH42 was modified by adding a C-terminal lysine for conjugation via the primary amine on its side chain. The alpha-NH2 was acetylated to prevent conjugation with functionalized LNP while the carboxy end was left unchanged with amide capping to maintain the physicochemical properties of the initially screened original peptide to react with primary amine on the lysine side chain, thereby directionally conjugating onto the surface of the LNP. Formulated nanoparticles were 91.6 nm in diameter with a PDI value less than 0.05. Encapsulation efficiency was 98.5%. At 48-hours post-subretinal delivery of 50 μg GFP mRNA in a 100 μl volume, wide field fundus autofluorescence images showed GFP expression within the margins of the bleb (FIG. 7A) Confocal images of retinal cross-sections immunolabeled with an antibody specific to GFP showed robust expression in the PRs and RPE throughout the bleb (FIG. 7B). H & E imaging demonstrated re-attachment of the neural retina to the RPE within the bleb, 48 hours post-injection (FIG. 7B). Further staining showed GFP expression co-labeled with cone arrestin, rod arrestin, RPE65 and glutamine synthetase, verifying expression was localized to PRs, RPE and Müller glia, respectively (FIGS. 7C to 7F). Arrows highlight places of co-localization for each cell type. For both rod and cone arrestin, co-localization with GFP was observed in the synapses and inner segments. Additionally, the rod arrestin and glutamine synthetase antibodies labeled the cytoplasm of the cell bodies, while the GFP expression was observed in the nucleus of the cell bodies. Therefore, much of the co-localization has the appearance of the antibody surrounding the GFP expression (FIGS. 7D and 7E). The immune response utilizing CD3 and IBA-1 markers was evaluated. Imaging showed microglia activation and to a lesser degree, T-cell infiltration in the choroid, corroborating immune-related inflammation (FIG. 7G). These data provided additional evidence of successful delivery of mRNA to the neural retina with the utilization of 0.15%-MH42 peptide conjugated LNP.

### DISCUSSION

Gene therapies are quickly advancing to tackle genetic diseases of the retina as evidenced by recent landmark achievements such as the first FDA-approved gene therapy for a genetic condition or the first-in-human delivery of CRISPR/Cas components to the retina for in vivo gene editing. Concurrent with the success of mRNA vaccines, non-viral LNPs have gained enormous momentum in the gene editing field. For example, systemic administration of LNP-mRNA encoding for RNA-guided Cas9 has led to knockdown of misfolded transthyretin (TTR) protein in six amyloidosis patients, with over 90% reduction in TTR protein. LNP-encapsulated CRISPR/Cas components have also shown tolerability and preliminary efficacy in a clinical trial addressing hereditary angioedema after a single systemic administration (Intellia). In addition, LNPs have been deployed in primates to deliver mRNA-encoded nucleases for base editing of PCSK9, a protein implicated in LDL-cholesterol management tied to ischemic heart disease.

As LNPs have become the principal non-viral gene delivery vehicle, the present technology translates this platform into gene editing therapeutics for the diverse forms of IRD. The main limitation of LNPs with regards to retinal delivery is their inability to penetrate the neural retina. The inventors previously have shown that LNPs have restricted expression to the RPE when delivered subretinally, and after intravitreal delivery they get sequestered in the vitreous leading to minimal Müller glia expression. Targeting of the neural retina, and in particular the PRs, is required to advance LNP therapeutics for IRDs.

Results disclosed herein demonstrate that multiple rounds of in vivo bio-panning with a bacteriophage library containing 1.28×10⁹ unique peptides, elucidated 7mer peptide sequences that had increased binding affinity to 661w cone PR cells and increased PR localization after subretinal administration. Furthermore, conjugation methods allowed for the generation of peptide-conjugated LNPs with ideal size, encapsulation, and morphology. When injected subretinally, peptide-conjugated LNPs mediated expression in PRs, RPE and Müller glia in both rodents and non-human primates. To the inventors' knowledge, this is the first report showing LNP mediated transfection in the PRs, and successful translation to the non-human primate, which overall demonstrates the advancement of retinal LNP-mRNA delivery.

Although neuronal cells were not transfected from the vitreous, after subretinal delivery, successful transfection of the PRs was achieved. Results were consistent across species and conjugation strategies. First, PR localization of the TAMRA-dye conjugated peptides was observed in BALB/c mice. The optimal surface density of MH42 onto the LNPs was determined that lead to efficient gene delivery in Ai9 mouse. It is likely that intermediate surface density of these peptides provided either penetrative properties or interacted with still-undetermined cell receptors that mediated more efficient gene delivery; similar optimized MH42-LNP led to PR transfection in NHPs. In both the Ai9 mice and the NHP, the RPE and Müller glia were transfected as well. Most likely due to their highly phagocytic nature.

QikProp analysis showed that MH42 had the highest predicted potential for globular formation with low binding to serum albumin across peptides tested. It also possessed a histidine residue not found in the other candidates selected. PR transfection after subretinal delivery could have been a result of improved interaction with cell membranes or extracellular matrix leading to increased uptake and/or escape of nucleic acid cargo into the cytoplasmic compartment. Slightly basic amines have been postulated to be ideal in generating endosomal rupture events via proton sponge effect and osmotic swelling; therefore it is possible this peptide's physicochemical properties are aiding in release from endosomal vesicles and increased expression.

The immune response associated with the MH42-LNP delivery was assessed. In mice, areas of the retina were observed that had PR cell loss. These areas had disrupted/swollen RPE, a thinned outer nuclear layer and increased Müller glia transfection. Interestingly, in these same areas, there was no detectable T cell infiltration or microglia activation. It is likely that the PR cell loss in the rodents is due to dose related RPE toxicity from LNP overload/accumulation or robust tdtomato expression, which is known to be toxic. Once the RPE is compromised, they are unable to support the PRs, which leads to cell death. In contrast, in the NHP, PR cell loss was not observed, but in areas of disrupted RPE, there was T cell infiltration in the choroid and microglia activation in the retina. The NHP was dosed at a 100× higher concentration than the rodents, which could account for the observable immune response. In addition, NHPs are known to have increased immunoreactivity compared to rodents. The current LNP formulation contains the MC3 ionizable lipid which was developed for liver targeting and has been associated with increased toxicity as compared to biodegradable counterparts.

In conclusion, this study showed that peptide conjugated LNPs may be powerful tools to advance mRNA-based therapeutics in the retina. The translation of the results into the NHP eye, highlights the advancement of LNP-mRNA delivery toward clinical application for IRDs.

TABLE 1A
Properties of certain exemplary disclosed conjugates with
varying targeting ligand surface coating concentrations

| | | | |
|---|---|---|---|
| | TB01 | TB02 | TB03 |
| Column1 | Unconjugated | 10% conjugated | 20% conjugated |

Properties
Encapsulation Efficiency

| | | | |
|---|---|---|---|
| Uncapsulated Signal | 346.3333333 | 789 | 956.6666667 |
| Total Signal 1 | 11003 | 11559 | 11666 |
| Total Signal 2 | 10918 | 11506 | 11654 |
| Total Signal 3 | 10316 | 10970 | 11065 |
| Encapsulation 1 (%) | 96.8523736 | 93.17415001 | 91.7995314 |
| Encapsulation 2 (%) | 96.82786835 | 93.14270815 | 91.79108747 |
| Encapsulation 3 (%) | 96.64275559 | 92.80765725 | 91.3541196 |
| Encapsulation (%, AV ± SD) | 96.8 ± 0.1 | 93 ± 0.2 | 91.6 ± 0.3 |

Concentration

| Conc. of encapsulated RNA | 0.635622466 | 0.645198161 | 0.642080967 |
|---|---|---|---|
| in sample well (μg/ml) | | | |
| Conc. of encapsulated RNA | 381.3734794 | 387.1188969 | 385.2485801 |
| in tube (μg/ml) | | | |
| | TB04 | TB05 | TB06 |
| Column1 | 40% conjugated | 60% conjugated | 80% conjugated |

Properties
Encapsulation Efficiency

| | | | |
|---|---|---|---|
| Uncapsulated Signal | 990.3333333 | 866.3333333 | 1514.666667 |
| Total Signal 1 | 13142 | 8199 | 3672 |
| Total Signal 2 | 13003 | 8199 | 3544 |
| Total Signal 3 | 12070 | 7986 | 3576 |
| Encapsulation 1 (%) | 92.46436362 | 89.43367077 | 58.75090777 |
| Encapsulation 2 (%) | 92.38380886 | 89.43367077 | 57.26109857 |
| Encapsulation 3 (%) | 91.79508423 | 89.15184907 | 57.64354959 |
| Encapsulation (%, AV ± SD) | 92.2 ± 0.4 | 89.3 ± 0.2 | 57.9 ± 0.8 |

Concentration

| | | | |
|---|---|---|---|
| Conc. of encapsulated RNA | 0.718054945 | 0.443843689 | 0.127295633 |
| in sample well (μg/ml) | | | |
| Conc. of encapsulated RNA | 430.8329671 | 266.3062135 | 76.37737993 |
| in tube (μg/ml) | | | |

### Pharmaceutically Relevant Properties and 3D Superposition of Peptide Sequences Tested

Following the encouraging results after intravitreal and subretinal delivery, the peptide sequences 3D structure was analyzed, as well as their amino acid composition in detail using Molecular Operating Environment (MOE) software and computed relevant physicochemical properties of these using the QikProp module of Schrodinger drug discovery suite. For this analysis, the focus was primarily on the top 5 performers' sequences from the in vivo isolated peptides while highlighting those that were tested against target cells such as 661w cone photoreceptors and RPE, and thoroughly evaluated in vivo on surface of mRNA-loaded LNPs (Tables 1 and 2 to 5, and FIGS. 2D and 2C).

TABLE 2
661w Top Five Peptide Candidates Sequence Characteristics

| | | | | pI |
|---|---|---|---|---|
| Peptide | One Letter | | | (Isoelectric |
| Number | Code | Linker Seq. | Color | point) |
| 42 | SPALHFL | GGGS[C] | Orange | 6.63 |
| | (SEQ ID No. 23) | (SEQ ID No. 35 [SEQ ID No. 52]) | | |
| 43 | SNLAAFP | GGGS[C] | Cyan | 5.02 |
| | (SEQ ID No. 24) | (SEQ ID No. 35 [SEQ ID No. 52]) | | |
| 50 | MPVAVYR | GGGS[C] | Blue | 7.25 |
| | (SEQ ID No. 26) | (SEQ ID No. 35 [SEQ ID No. 52]) | | |
| 52 | LAFHRMP | GGGS[C] | Red | 7.4 |
| | (SEQ ID No. 27) | (SEQ ID No. 35 [SEQ ID No. 52]) | | |
| 54 | HLSSLTP | GGGS[C] | Yellow | 6.63 |
| | (SEQ ID No. 28) | (SEQ ID No. 35 [SEQ ID No. 52]) | | |

TABLE 3
Peptide Characteristics

| Peptide Name | Acidic | Basic | Neutral | Hydrophobic |
|---|---|---|---|---|
| MH42 | 0% | 9% | 45% | 45% |
| (SEQ ID No. 32) | | | | |
| MH43 | 0% | 0% | 55% | 45% |
| (SEQ ID No. 33) | | | | |
| MH50 | 0% | 9% | 36% | 55% |
| (SEQ ID No. 34) | | | | |

TABLE 4
ARPE19 Cells Top Five Peptide Candidates Sequence Characteristics

| Peptide | One Letter | | | pI (Isoelectric |
|---|---|---|---|---|
| Number | Code | Linker Seq. | Color | point) |
| 3 | SPALHFL | GGGS[C] | Magenta | 7.25 |
| | (SEQ ID No. 23) | (SEQ ID No. 35 [SEQ ID No. 52]) | | |
| 50 | MPVAVYR | GGGS[C] | Blue | 7.25 |
| | (SEQ ID No. 26) | (SEQ ID No. 35 [SEQ ID No. 52]) | | |
| 52 | LAFHRMP | GGGS[C] | Red | 7.40 |
| | (SEQ ID No. 27) | (SEQ ID No. 35 [SEQ ID No. 52]) | | |
| 54 | HLSSLTP | GGGS[C] | Yellow | 6.63 |
| | (SEQ ID No. 28) | (SEQ ID No. 35 [SEQ ID No. 52]) | | |
| 57 | ATGPRSV | GGGS[C] | Lime | 7.25 |
| | (SEQ ID No. 29) | (SEQ ID No. 35 [SEQ ID No. 52]) | | |

TABLE 5
Peptide Characteristics

| Peptide Name | Acidic | Basic | Neutral | Hydrophobic |
|---|---|---|---|---|
| MH3 | 8% | 17% | 67% | 8% |
| (SEQ ID No. 31) | | | | |
| MH50 | 0% | 9% | 36% | 55% |
| (SEQ ID No. 34) | | | | |

For 661w top performers, the structural superposition of physiologically relevant conformations was elucidated and showed a striking similarity in 3D models across all five peptides, possibly hinting at conserved features that aid in specific cell membrane recognition or a common receptor target among these (FIG. 2D)). These conserved structural patterns were not present when examining the 3D superposition of the top 5 performing peptides for RPE cells, in fact, a rather unrelated and disordered conformation was seen among the top candidates for these cells when looking at their 3D spatial superposition (FIG. 2C). Without being bound to a particular theory, this could be due to increased heterogeneity of ARPE19 cell membranes compared to 661w cone photoreceptor cell line, leading to diverse peptide motifs being elucidated in the cell-ELISA screen as binders. It was also observed that for the peptides synthesized and tested in vitro and in vivo, a general trend of mostly neutral, slightly basic amino acid residues making up the targeting moieties with the exception of the poor performer, MH3 which contained a slightly more acidic conformation compared to the other candidate peptides (Tables 3 and 5). Additionally, the pharmaceutically pertinent physicochemical properties were computed for all peptide sequences isolated in this study (Tables 6 to 8).

TABLE 6
Certain computed physicochemical properties for some of the peptide sequences
disclosed herein

| SEQ ID | Molecule | Sequence | #stars | #rotor | mol_MW | #rtvFG |
|---|---|---|---|---|---|---|
| SEQ ID No. 1 | Peptide 1 | HILNWPT | 17 | 25 | 879.027 | 0 |
| SEQ ID No. 2 | Peptide 3 | DGPPRKP | 18 | 23 | 764.88 | 0 |
| SEQ ID No. 3 | Peptide 11 | HLSSIPG | 13 | 23 | 708.813 | 0 |
| SEQ ID No. 4 | Peptide 12 | FPHILAP | 12 | 20 | 792.977 | 0 |
| SEQ ID No. 5 | Peptide 13 | HVSPLNW | 17 | 24 | 850.973 | 0 |
| SEQ ID No. 6 | Peptide 14 | VFPMPPM | 10 | 20 | 817.072 | 0 |
| SEQ ID No. 7 | Peptide 15 | NVDNYPH | 17 | 25 | 856.891 | 0 |
| SEQ ID No. 8 | Peptide 16 | YPQGPFP | 18 | 19 | 803.914 | 0 |
| SEQ ID No. 9 | Peptide 18 | AFAPFKT | 14 | 24 | 779.935 | 0 |
| SEQ ID No. 10 | Peptide 20 | YFPAFTS | 13 | 24 | 830.936 | 0 |
| SEQ ID No. 11 | Peptide 21 | IYAKAPI | 15 | 25 | 773.971 | 0 |
| SEQ ID No. 12 | Peptide 22 | SQTNAIH | 17 | 27 | 768.826 | 0 |
| SEQ ID No. 13 | Peptide 23 | LSINNVT | 16 | 27 | 758.871 | 0 |
| SEQ ID No. 14 | Peptide 24 | SSPAAHT | 13 | 21 | 668.706 | 0 |
| SEQ ID No. 15 | Peptide 25 | WGPPTLI | 12 | 20 | 781.951 | 0 |
| SEQ ID No. 16 | Peptide 26 | SIAHNTM | 17 | 27 | 771.887 | 0 |
| SEQ ID No. 17 | Peptide 27 | TVKTLTT | 15 | 30 | 761.914 | 0 |
| SEQ ID No. 18 | Peptide 29 | FIPNGSM | 16 | 24 | 763.908 | 0 |
| SEQ ID No. 19 | Peptide 34 | SLTNSSF | 15 | 28 | 753.808 | 0 |
| SEQ ID No. 20 | Peptide 35 | INLNNVT | 18 | 27 | 785.896 | 0 |
| SEQ ID No. 21 | Peptide 37 | ATKSYNT | 17 | 30 | 782.849 | 0 |
| SEQ ID No. 22 | Peptide 38 | TSMLPYR | 15 | 30 | 866.043 | 0 |
| SEQ ID No. 23 | Peptide 42 | SPALHFL | 14 | 23 | 782.938 | 0 |
| SEQ ID No. 24 | Peptide 43 | SNLAAFP | 14 | 21 | 717.821 | 0 |
| SEQ ID No. 25 | Peptide 44 | DVATNMH | 17 | 26 | 785.871 | 0 |
| SEQ ID No. 26 | Peptide 50 | MPVAVYR | 15 | 26 | 834.045 | 0 |
| SEQ ID No. 27 | Peptide 52 | LAFHRMP | 16 | 27 | 870.081 | 0 |
| SEQ ID No. 28 | Peptide 54 | HLSSLTP | 14 | 25 | 752.866 | 0 |
| SEQ ID No. 29 | Peptide 57 | ATGPRSV | 13 | 23 | 685.779 | 0 |
| SEQ ID No. 30 | Peptide 58 | NVSAYPT | 15 | 23 | 749.82 | 0 |
| SEQ ID No. 31 | MH3 | DGPPRKPGGGSC | 19 | 33 | 1144.174 | 1 |
| SEQ ID No. 32 | MH42 | SPALHFLGGGSC | 18 | 33 | 1162.232 | 1 |
| SEQ ID No. 33 | MH43 | SNLAAFPGGGSC | 19 | 31 | 1097.115 | 1 |
| SEQ ID No. 34 | MH50 | MPVAVYRGGGSC | 18 | 36 | 1213.339 | 1 |

| SEQ ID | SASA | FOSA | FISA | PISA | WPSA | volume |
|---|---|---|---|---|---|---|
| SEQ ID No. 1 | 1204.72 | 573.938 | 411.935 | 218.847 | 0 | 2511.696 |
| SEQ ID No. 2 | 1199.109 | 661.146 | 537.963 | 0 | 0 | 2280.482 |
| SEQ ID No. 3 | 1146.042 | 630.999 | 435.561 | 79.481 | 0 | 2164.745 |
| SEQ ID No. 4 | 1206.92 | 714.412 | 294.267 | 198.24 | 0 | 2410.981 |
| SEQ ID No. 5 | 1256.71 | 553.561 | 444.787 | 258.361 | 0 | 2484.927 |
| SEQ ID No. 6 | 1135.747 | 729.327 | 232.013 | 113.35 | 61.058 | 2387.162 |
| SEQ ID No. 7 | 1118.576 | 403.415 | 535.341 | 179.821 | 0 | 2287.772 |
| SEQ ID No. 8 | 1139.969 | 515.875 | 390.556 | 233.538 | 0 | 2303.168 |
| SEQ ID No. 9 | 1124.733 | 547.271 | 328.518 | 248.944 | 0 | 2296.907 |
| SEQ ID No. 10 | 1148.454 | 381.32 | 391.257 | 375.878 | 0 | 2378.181 |
| SEQ ID No. 11 | 1228.391 | 760.471 | 360.592 | 107.328 | 0 | 2421.527 |
| SEQ ID No. 12 | 1213.89 | 531.925 | 586.346 | 95.619 | 0 | 2296.796 |
| SEQ ID No. 13 | 1188.974 | 665.744 | 523.23 | 0 | 0 | 2308.076 |
| SEQ ID No. 14 | 1058.815 | 541.235 | 438.113 | 79.467 | 0 | 1977.432 |
| SEQ ID No. 15 | 1154.495 | 691.223 | 309.221 | 154.052 | 0 | 2314.266 |
| SEQ ID No. 16 | 1204.873 | 613.463 | 494.075 | 55.092 | 42.244 | 2291.87 |
| SEQ ID No. 17 | 1225.903 | 738.914 | 486.989 | 0 | 0 | 2383.649 |
| SEQ ID No. 18 | 1197.813 | 580.377 | 457.293 | 117.822 | 42.321 | 2289.428 |
| SEQ ID No. 19 | 1196.842 | 486.49 | 532.976 | 177.376 | 0 | 2261.873 |
| SEQ ID No. 20 | 1207.484 | 642.517 | 564.967 | 0 | 0 | 2370.159 |
| SEQ ID No. 21 | 1148.672 | 469.896 | 585.477 | 93.299 | 0 | 2259.065 |
| SEQ ID No. 22 | 1322.18 | 659.512 | 519.377 | 100.849 | 42.441 | 2580.87 |
| SEQ ID No. 23 | 1203.792 | 650.252 | 345.932 | 207.609 | 0 | 2375.585 |
| SEQ ID No. 24 | 1138.312 | 583.496 | 405.857 | 148.959 | 0 | 2187.976 |
| SEQ ID No. 25 | 1215.59 | 551.179 | 525.69 | 95.321 | 43.401 | 2322.168 |
| SEQ ID No. 26 | 1345.901 | 751.321 | 440.792 | 111.743 | 42.045 | 2573.388 |
| SEQ ID No. 27 | 1296.675 | 625.55 | 454.616 | 180.184 | 36.325 | 2592.303 |
| SEQ ID No. 28 | 1142.332 | 639.655 | 418.633 | 84.043 | 0 | 2259.067 |
| SEQ ID No. 29 | 1146.52 | 642.003 | 504.517 | 0 | 0 | 2125.697 |
| SEQ ID No. 30 | 1088.087 | 509.469 | 470.826 | 107.793 | 0 | 2143.761 |
| SEQ ID No. 31 | 1657.49 | 827.263 | 761.586 | 0 | 68.64 | 3090.791 |
| SEQ ID No. 32 | 1655.179 | 802.273 | 579 | 205.171 | 68.735 | 3188 |
| SEQ ID No. 33 | 1610.141 | 764.962 | 634.137 | 142.311 | 68.732 | 3001.87 |
| SEQ ID No. 34 | 1754.232 | 893.76 | 636.179 | 113.438 | 110.855 | 3336.732 |

TABLE 7
Additional computed physicochemical properties for some of the peptide sequences
disclosed herein

| SEQ ID | Molecule | Sequence | donorHB | accptHB | glob | QPpolrz |
|---|---|---|---|---|---|---|
| SEQ ID No. 1 | Peptide 1 | HILNWPT | 9 | 20.2 | 0.7417494 | 80.402 |
| SEQ ID No. 2 | Peptide 3 | DGPPRKP | 10.75 | 21.75 | 0.6987539 | 70.343 |
| SEQ ID No. 3 | Peptide 11 | HLSSIPG | 6.25 | 18.65 | 0.706159 | 66.47 |
| SEQ ID No. 4 | Peptide 12 | FPHILAP | 6 | 18.5 | 0.7204701 | 79.385 |
| SEQ ID No. 5 | Peptide 13 | HVSPLNW | 9 | 20.2 | 0.7060019 | 80.35 |
| SEQ ID No. 6 | Peptide 14 | VFPMPPM | 4.75 | 18.75 | 0.7605683 | 77.621 |
| SEQ ID No. 7 | Peptide 15 | NVDNYPH | 10.75 | 23.5 | 0.7506565 | 71.067 |
| SEQ ID No. 8 | Peptide 16 | YPQGPFP | 7.75 | 21 | 0.7398704 | 76.049 |
| SEQ ID No. 9 | Peptide 18 | AFAPFKT | 7.25 | 16.95 | 0.7485337 | 72.735 |
| SEQ ID No. 10 | Peptide 20 | YFPAFTS | 6.25 | 17.4 | 0.7502647 | 77.2 |
| SEQ ID No. 11 | Peptide 21 | IYAKAPI | 8.25 | 17 | 0.7099394 | 75.728 |
| SEQ ID No. 12 | Peptide 22 | SQTNAIH | 10.25 | 22.15 | 0.693533 | 69.34 |
| SEQ ID No. 13 | Peptide 23 | LSINNVT | 9 | 19.9 | 0.7103835 | 68.878 |
| SEQ ID No. 14 | Peptide 24 | SSPAAHT | 6.25 | 19.35 | 0.7195807 | 60.258 |
| SEQ ID No. 15 | Peptide 25 | WGPPTLI | 6 | 17.2 | 0.7329063 | 75.092 |
| SEQ ID No. 16 | Peptide 26 | SIAHNTM | 8.25 | 20.15 | 0.6977242 | 68.756 |
| SEQ ID No. 17 | Peptide 27 | TVKTLTT | 7.5 | 17.8 | 0.7039425 | 69.977 |
| SEQ ID No. 18 | Peptide 29 | FIPNGSM | 7 | 18.7 | 0.7013379 | 71.184 |
| SEQ ID No. 19 | Peptide 34 | SLTNSSF | 7.25 | 19.05 | 0.6962636 | 68.081 |
| SEQ ID No. 20 | Peptide 35 | INLNNVT | 10.75 | 21.45 | 0.7119813 | 71.363 |
| SEQ ID No. 21 | Peptide 37 | ATKSYNT | 10.25 | 20.1 | 0.7248612 | 65.882 |
| SEQ ID No. 22 | Peptide 38 | TSMLPYR | 10.25 | 19.9 | 0.6882062 | 78.833 |
| SEQ ID No. 23 | Peptide 42 | SPALHFL | 6.25 | 17.95 | 0.7152544 | 76.131 |
| SEQ ID No. 24 | Peptide 43 | SNLAAFP | 7 | 18.2 | 0.7160317 | 69.347 |
| SEQ ID No. 25 | Peptide 44 | DVATNMH | 8.25 | 20.45 | 0.6976543 | 70.995 |
| SEQ ID No. 26 | Peptide 50 | MPVAVYR | 10.25 | 18.5 | 0.6747697 | 81.206 |
| SEQ ID No. 27 | Peptide 52 | LAFHRMP | 10.25 | 19.75 | 0.7038138 | 81.974 |
| SEQ ID No. 28 | Peptide 54 | HLSSLTP | 6.25 | 19.35 | 0.7288846 | 69.004 |
| SEQ ID No. 29 | Peptide 57 | ATGPRSV | 9.25 | 18.65 | 0.6973502 | 64.148 |
| SEQ ID No. 30 | Peptide 58 | NVSAYPT | 7.25 | 18.9 | 0.7389568 | 65.9 |
| SEQ ID No. 31 | MH3 | DGPPRKPGGGSC | 11.75 | 28.25 | 0.6190997 | 96.35 |
| SEQ ID No. 32 | MH42 | SPALHFLGGGSC | 7.25 | 24.45 | 0.632896 | 102.2 |
| SEQ ID No. 33 | MH43 | SNLAAFPGGGSC | 8 | 24.7 | 0.6250226 | 95.435 |
| SEQ ID No. 34 | MH50 | MPVAVYRGGGSC | 11.25 | 25 | 0.6155911 | 105.35 |

| SEQ ID | QPlogPC16 | QPlogPoct | QPlogPw | QPlogPo/w | QPlogS |
|---|---|---|---|---|---|
| SEQ ID No. 1 | 27.973 | 53.253 | 45.383 | −2.75 | 0.285 |
| SEQ ID No. 2 | 26.711 | 52.588 | 48.998 | −7.52 | 1.098 |
| SEQ ID No. 3 | 24.328 | 43.091 | 39.759 | −3.562 | 0.304 |
| SEQ ID No. 4 | 25.946 | 46.202 | 37.825 | −0.83 | 2.131 |
| SEQ ID No. 5 | 29.087 | 52.828 | 46.768 | −3.198 | −0.41 |
| SEQ ID No. 6 | 24.052 | 44.422 | 34.045 | −0.237 | −1.73 |
| SEQ ID No. 7 | 26.956 | 55.143 | 53.509 | −8.403 | 2 |
| SEQ ID No. 8 | 25.96 | 50.125 | 46.704 | −3.917 | 0.91 |
| SEQ ID No. 9 | 25.304 | 45.446 | 38.346 | −1.764 | 1.006 |
| SEQ ID No. 10 | 27.051 | 47.535 | 38.815 | −1.032 | −0.495 |
| SEQ ID No. 11 | 26.618 | 47.186 | 40.323 | −1.7 | 0.242 |
| SEQ ID No. 12 | 27.883 | 52.454 | 53.023 | −7.094 | 2 |
| SEQ ID No. 13 | 26.185 | 49.145 | 46.109 | −5.589 | 1.76 |
| SEQ ID No. 14 | 22.399 | 41.368 | 39.3 | −4.921 | 0.873 |
| SEQ ID No. 15 | 24.657 | 44.379 | 36.717 | −1.03 | −1.491 |
| SEQ ID No. 16 | 26.646 | 47.799 | 45.223 | −4.635 | 1.204 |
| SEQ ID No. 17 | 26.398 | 46.074 | 38.148 | −3.112 | 0.824 |
| SEQ ID No. 18 | 26.438 | 46.304 | 44.265 | −3.54 | 0.922 |
| SEQ ID No. 19 | 27.024 | 46.362 | 43.462 | −4.512 | 1.219 |
| SEQ ID No. 20 | 27.379 | 53.585 | 51.969 | −7.021 | 2 |
| SEQ ID No. 21 | 26.833 | 50.878 | 47.351 | −6.346 | 2 |
| SEQ ID No. 22 | 30.391 | 54.121 | 44.253 | −2.716 | −1.555 |
| SEQ ID No. 23 | 26.249 | 45.562 | 37.595 | −1.226 | −1.655 |
| SEQ ID No. 24 | 24.78 | 44.773 | 43.852 | −3.683 | 1.257 |
| SEQ ID No. 25 | 27.431 | 49.417 | 45.79 | −6.096 | 0.089 |
| SEQ ID No. 26 | 30.244 | 52.803 | 43.142 | −1.713 | −3.063 |
| SEQ ID No. 27 | 30.116 | 54.848 | 45.482 | −2.025 | −1.365 |
| SEQ ID No. 28 | 24.602 | 44.717 | 38.769 | −3.091 | 0.388 |
| SEQ ID No. 29 | 24.77 | 46.384 | 43.382 | −4.897 | 0.308 |
| SEQ ID No. 30 | 24.189 | 45.544 | 41.778 | −4.687 | 1.334 |
| SEQ ID No. 31 | 39.044 | 68.097 | 67.848 | −9.533 | 2 |
| SEQ ID No. 32 | 38.4 | 61.374 | 56.292 | −3.137 | −0.693 |
| SEQ ID No. 33 | 37.214 | 60.185 | 60.745 | −5.618 | 1.171 |
| SEQ ID No. 34 | 41.531 | 67.656 | 60.646 | −3.62 | −1.8 |

TABLE 8
Additional computed physicochemical properties for some of the peptide sequences
disclosed herein

| SEQ ID | Molecule | Sequence | QPlogBB | #metab | QPlogKhsa |
|---|---|---|---|---|---|
| SEQ ID No. 1 | Peptide 1 | HILNWPT | −5.536 | 15 | −2.611 |
| SEQ ID No. 2 | Peptide 3 | DGPPRKP | −7.371 | 14 | −3.377 |
| SEQ ID No. 3 | Peptide 11 | HLSSIPG | −6.247 | 13 | −2.705 |
| SEQ ID No. 4 | Peptide 12 | FPHILAP | −4.023 | 12 | −1.913 |
| SEQ ID No. 5 | Peptide 13 | HVSPLNW | −6.434 | 15 | −2.736 |
| SEQ ID No. 6 | Peptide 14 | VFPMPPM | −2.767 | 11 | −1.886 |
| SEQ ID No. 7 | Peptide 15 | NVDNYPH | −6.879 | 18 | −4.144 |
| SEQ ID No. 8 | Peptide 16 | YPQGPFP | −4.94 | 15 | −2.902 |
| SEQ ID No. 9 | Peptide 18 | AFAPFKT | −3.995 | 15 | −2.099 |
| SEQ ID No. 10 | Peptide 20 | YFPAFTS | −5.118 | 16 | −1.916 |
| SEQ ID No. 11 | Peptide 21 | IYAKAPI | −4.916 | 14 | −2.014 |
| SEQ ID No. 12 | Peptide 22 | SQTNAIH | −8.792 | 17 | −4.042 |
| SEQ ID No. 13 | Peptide 23 | LSINNVT | −7.578 | 16 | −3.532 |
| SEQ ID No. 14 | Peptide 24 | SSPAAHT | −5.945 | 14 | −3.134 |
| SEQ ID No. 15 | Peptide 25 | WGPPTLI | −4.081 | 12 | −1.892 |
| SEQ ID No. 16 | Peptide 26 | SIAHNTM | −7.328 | 15 | −3.28 |
| SEQ ID No. 17 | Peptide 27 | TVKTLTT | −6.999 | 16 | −2.411 |
| SEQ ID No. 18 | Peptide 29 | FIPNGSM | −6.579 | 14 | −2.833 |
| SEQ ID No. 19 | Peptide 34 | SLTNSSF | −8.055 | 17 | −3.155 |
| SEQ ID No. 20 | Peptide 35 | INLNNVT | −8.096 | 17 | −4.063 |
| SEQ ID No. 21 | Peptide 37 | ATKSYNT | −7.876 | 19 | −3.512 |
| SEQ ID No. 22 | Peptide 38 | TSMLPYR | −8.053 | 15 | −2.543 |
| SEQ ID No. 23 | Peptide 42 | SPALHFL | −4.932 | 13 | −2.054 |
| SEQ ID No. 24 | Peptide 43 | SNLAAFP | −5.581 | 14 | −2.823 |
| SEQ ID No. 25 | Peptide 44 | DVATNMH | −7.699 | 15 | −3.379 |
| SEQ ID No. 26 | Peptide 50 | MPVAVYR | −6.956 | 13 | −2.202 |
| SEQ ID No. 27 | Peptide 52 | LAFHRMP | −6.695 | 13 | −2.317 |
| SEQ ID No. 28 | Peptide 54 | HLSSLTP | −5.798 | 14 | −2.632 |
| SEQ ID No. 29 | Peptide 57 | ATGPRSV | −7.341 | 13 | −2.992 |
| SEQ ID No. 30 | Peptide 58 | NVSAYPT | −6.173 | 16 | −3.076 |
| SEQ ID No. 31 | MH3 | DGPPRKPGGGSC | −13.707 | 19 | −4.705 |
| SEQ ID No. 32 | MH42 | SPALHFLGGGSC | −10.495 | 18 | −3.222 |
| SEQ ID No. 33 | MH43 | SNLAAFPGGGSC | −11.56 | 19 | −4.048 |
| SEQ ID No. 34 | MH50 | MPVAVYRGGGSC | −12.128 | 18 | −3.456 |

| | | | | | | | |
|---|---|---|---|---|---|---|---|
| SEQ ID | PSA | QPlogHERG | #ringa | #in34 | #in56 | [symbol] | [symbol] |

| | | | | | | | |
|---|---|---|---|---|---|---|---|
| SEQ ID No. 1 | 350.44 | 3.515 | 19 | 0 | 19 | 4 | 63 |
| SEQ ID No. 2 | 377.94 | 3.57 | 15 | 0 | 15 | 12 | 54 |
| SEQ ID No. 3 | 330.12 | 2.229 | 10 | 0 | 10 | 4 | 50 |
| SEQ ID No. 4 | 269.19 | 1.894 | 21 | 0 | 21 | 8 | 57 |
| SEQ ID No. 5 | 361.53 | 2.748 | 19 | 0 | 19 | 4 | 61 |
| SEQ ID No. 6 | 232.3 | 2.948 | 21 | 0 | 21 | 12 | 56 |
| SEQ ID No. 7 | 420.76 | 7.027 | 16 | 0 | 16 | 4 | 61 |
| SEQ ID No. 8 | 311.04 | 3.414 | 27 | 0 | 27 | 12 | 58 |
| SEQ ID No. 9 | 282.8 | 1.38 | 17 | 0 | 17 | 4 | 56 |
| SEQ ID No. 10 | 313.61 | 1.529 | 23 | 0 | 23 | 4 | 60 |
| SEQ ID No. 11 | 307.01 | 1.431 | 11 | 0 | 11 | 4 | 55 |
| SEQ ID No. 12 | 437.25 | 4.696 | 5 | 0 | 5 | 0 | 54 |
| SEQ ID No. 13 | 404.16 | 5.425 | 0 | 0 | 0 | 0 | 53 |
| SEQ ID No. 14 | 342.38 | 2.446 | 10 | 0 | 10 | 4 | 47 |
| SEQ ID No. 15 | 279.79 | 2.306 | 19 | 0 | 19 | 8 | 56 |
| SEQ ID No. 16 | 382.98 | 3.583 | 5 | 0 | 5 | 0 | 53 |
| SEQ ID No. 17 | 368.14 | 1.846 | 0 | 0 | 0 | 0 | 53 |
| SEQ ID No. 18 | 329.66 | 3.342 | 11 | 0 | 11 | 4 | 53 |
| SEQ ID No. 19 | 403.12 | 2.976 | 6 | 0 | 6 | 0 | 53 |
| SEQ ID No. 20 | 432.21 | 6.825 | 0 | 0 | 0 | 0 | 55 |
| SEQ ID No. 21 | 427.66 | 3.114 | 6 | 0 | 6 | 0 | 55 |
| SEQ ID No. 22 | 381.6 | 1.837 | 11 | 0 | 11 | 4 | 60 |
| SEQ ID No. 23 | 303.56 | 1.765 | 16 | 0 | 16 | 4 | 56 |
| SEQ ID No. 24 | 332.87 | 3.432 | 11 | 0 | 11 | 4 | 51 |
| SEQ ID No. 25 | 406.74 | 5.198 | 5 | 0 | 5 | 0 | 54 |
| SEQ ID No. 26 | 344.71 | 1.567 | 11 | 0 | 11 | 4 | 58 |
| SEQ ID No. 27 | 356.16 | 1.711 | 16 | 0 | 16 | 4 | 61 |
| SEQ ID No. 28 | 341.51 | 2.575 | 10 | 0 | 10 | 4 | 53 |
| SEQ ID No. 29 | 373.95 | 2.465 | 5 | 0 | 5 | 4 | 48 |
| SEQ ID No. 30 | 358.5 | 3.999 | 11 | 0 | 11 | 4 | 53 |
| SEQ ID No. 31 | 530.95 | 7.778 | 15 | 0 | 15 | 12 | 72 |
| SEQ ID No. 32 | 450.35 | 5.942 | 16 | 0 | 16 | 4 | 74 |
| SEQ ID No. 33 | 484.35 | 7.488 | 11 | 0 | 11 | 4 | 69 |
| SEQ ID No. 34 | 488.99 | 6.099 | 11 | 0 | 11 | 4 | 76 |

[symbol]

indicates data missing or illegible when filed

TABLE 9
Schrodinger ™ QikProp output values for pharmaceutically relevant properties for
peptide sequences isolated. Peptides MH3, MH42, MH43, and MH50 are with added linker.

| Molecule | Sequence | #rotor | mol_MW | #rtvFG | SASA | FOSA |
|---|---|---|---|---|---|---|
| Peptide 1 | HILNWPT | 25 | 879.027 | 0 | 1204.72 | 573.938 |
| Peptide 3 | DGPPRKP | 23 | 764.88 | 0 | 1199.109 | 661.146 |
| Peptide 11 | HLSSIPG | 23 | 708.813 | 0 | 1146.042 | 630.999 |
| Peptide 12 | FPHILAP | 20 | 792.977 | 0 | 1206.92 | 714.412 |
| Peptide 13 | HVSPLNW | 24 | 850.973 | 0 | 1256.71 | 553.561 |
| Peptide 14 | VFPMPPM | 20 | 817.072 | 0 | 1135.747 | 729.327 |
| Peptide 15 | NVDNYPH | 25 | 856.891 | 0 | 1118.576 | 403.415 |
| Peptide 16 | YPQGPFP | 19 | 803.914 | 0 | 1139.969 | 515.875 |
| Peptide 18 | AFAPFKT | 24 | 779.935 | 0 | 1124.733 | 547.271 |
| Peptide 20 | YFPAFTS | 24 | 830.936 | 0 | 1148.454 | 381.32 |
| Peptide 21 | IYAKAPI | 25 | 773.971 | 0 | 1228.391 | 760.471 |
| Peptide 22 | SQTNAIH | 27 | 768.826 | 0 | 1213.89 | 531.925 |
| Peptide 23 | LSINNVT | 27 | 758.871 | 0 | 1188.974 | 665.744 |
| Peptide 24 | SSPAAHT | 21 | 668.706 | 0 | 1058.815 | 541.235 |
| Peptide 25 | WGPPTLI | 20 | 781.951 | 0 | 1154.495 | 691.223 |
| Peptide 26 | SIAHNTM | 27 | 771.887 | 0 | 1204.873 | 613.463 |
| Peptide 27 | TVKTLTT | 30 | 761.914 | 0 | 1225.903 | 738.914 |
| Peptide 29 | FIPNGSM | 24 | 763.908 | 0 | 1197.813 | 580.377 |
| Peptide 34 | SLTNSSF | 28 | 753.808 | 0 | 1196.842 | 486.49 |
| Peptide 35 | INLNNVT | 27 | 785.896 | 0 | 1207.484 | 642.517 |
| Peptide 37 | ATKSYNT | 30 | 782.849 | 0 | 1148.672 | 469.896 |
| Peptide 38 | TSMLPYR | 30 | 866.043 | 0 | 1322.18 | 659.512 |
| Peptide 42 | SPALHFL | 23 | 782.938 | 0 | 1203.792 | 650.252 |
| Peptide 43 | SNLAAFP | 21 | 717.821 | 0 | 1138.312 | 583.496 |
| Peptide 44 | DVATNMH | 26 | 785.871 | 0 | 1215.59 | 551.179 |
| Peptide 50 | MPVAVYR | 26 | 834.045 | 0 | 1345.901 | 751.321 |
| Peptide 52 | LAFHRMP | 27 | 870.081 | 0 | 1296.675 | 625.55 |
| Peptide 54 | HLSSLTP | 25 | 752.866 | 0 | 1142.332 | 639.655 |
| Peptide 57 | ATGPRSV | 23 | 685.779 | 0 | 1146.52 | 642.003 |
| Peptide 58 | NVSAYPT | 23 | 749.82 | 0 | 1088.087 | 509.469 |
| MH3 | DGPPRKPGGGSC | 33 | 1144.174 | 1 | 1657.49 | 827.263 |
| MH42 | SPALHFLGGGSC | 33 | 1162.232 | 1 | 1655.179 | 802.273 |
| MH43 | SNLAAFPGGGSC | 31 | 1097.115 | 1 | 1610.141 | 764.962 |
| MH50 | MPVAVYRGGGSC | 36 | 1213.339 | 1 | 1754.232 | 893.76 |

| Molecule | FISA | glob | QPpolrz | QPlogPC16 | QPlogPOCT |
|---|---|---|---|---|---|
| Peptide 1 | 411.935 | 0.7417494 | 80.402 | 27.973 | 53.253 |
| Peptide 3 | 537.963 | 0.6987539 | 70.343 | 26.711 | 52.588 |
| Peptide 11 | 435.561 | 0.706159 | 66.47 | 24.328 | 43.091 |
| Peptide 12 | 294.267 | 0.7204701 | 79.385 | 25.946 | 46.202 |
| Peptide 13 | 444.787 | 0.7060019 | 80.35 | 29.087 | 52.828 |
| Peptide 14 | 232.013 | 0.7605683 | 77.621 | 24.052 | 44.422 |
| Peptide 15 | 535.341 | 0.7506565 | 71.067 | 26.956 | 55.143 |
| Peptide 16 | 390.556 | 0.7398704 | 76.049 | 25.96 | 50.125 |
| Peptide 18 | 328.518 | 0.7485337 | 72.735 | 25.304 | 45.446 |
| Peptide 20 | 391.257 | 0.7502647 | 77.2 | 27.051 | 47.535 |
| Peptide 21 | 360.592 | 0.7099394 | 75.728 | 26.618 | 47.186 |
| Peptide 22 | 586.346 | 0.693533 | 69.34 | 27.883 | 52.454 |
| Peptide 23 | 523.23 | 0.7103835 | 68.878 | 26.185 | 49.145 |
| Peptide 24 | 438.113 | 0.7195807 | 60.258 | 22.399 | 41.368 |
| Peptide 25 | 309.221 | 0.7329063 | 75.092 | 24.657 | 44.379 |
| Peptide 26 | 494.075 | 0.6977242 | 68.756 | 26.646 | 47.799 |
| Peptide 27 | 486.989 | 0.7039425 | 69.977 | 26.398 | 46.074 |
| Peptide 29 | 457.293 | 0.7013379 | 71.184 | 26.438 | 46.304 |
| Peptide 34 | 532.976 | 0.6962636 | 68.081 | 27.024 | 46.362 |
| Peptide 35 | 564.967 | 0.7119813 | 71.363 | 27.379 | 53.585 |
| Peptide 37 | 585.477 | 0.7248612 | 65.882 | 26.833 | 50.878 |
| Peptide 38 | 519.377 | 0.6882062 | 78.833 | 30.391 | 54.121 |
| Peptide 42 | 345.932 | 0.7152544 | 76.131 | 26.249 | 45.562 |
| Peptide 43 | 405.857 | 0.7160317 | 69.347 | 24.78 | 44.773 |
| Peptide 44 | 525.69 | 0.6976543 | 70.995 | 27.431 | 49.417 |
| Peptide 50 | 440.792 | 0.6747697 | 81.206 | 30.244 | 52.803 |
| Peptide 52 | 454.616 | 0.7038138 | 81.974 | 30.116 | 54.848 |
| Peptide 54 | 418.633 | 0.7288846 | 69.004 | 24.602 | 44.717 |
| Peptide 57 | 504.517 | 0.6973502 | 64.148 | 24.77 | 46.384 |
| Peptide 58 | 470.826 | 0.7389568 | 65.9 | 24.189 | 45.544 |
| MH3 | 761.586 | 0.6190997 | 96.35 | 39.044 | 68.097 |
| MH42 | 579 | 0.632896 | 102.2 | 38.4 | 61.374 |
| MH43 | 634.137 | 0.6250226 | 95.435 | 37.214 | 60.185 |
| MH50 | 636.179 | 0.6155911 | 105.35 | 41.531 | 67.656 |

| Molecule | QPlogPw | QPlogPo/w | QplogS | QPlogBB | QPlogKhsa |
|---|---|---|---|---|---|
| Peptide 1 | 45.383 | −2.75 | 0.285 | −5.536 | −2.611 |
| Peptide 3 | 48.998 | −7.52 | 1.098 | −7.371 | −3.377 |
| Peptide 11 | 39.759 | −3.562 | 0.304 | −6.247 | −2.705 |
| Peptide 12 | 37.825 | 0.83 | −2.131 | −4.023 | −1.913 |
| Peptide 13 | 46.768 | −3.198 | −0.41 | −6.434 | −2.736 |
| Peptide 14 | 34.045 | −0.237 | −1.73 | −2.767 | −1.886 |
| Peptide 15 | 53.509 | −8.403 | 2 | −6.879 | −4.144 |
| Peptide 16 | 46.704 | −3.917 | 0.91 | −4.94 | −2.902 |
| Peptide 18 | 38.346 | −1.764 | 1.006 | −3.995 | −2.099 |
| Peptide 20 | 38.815 | −1.032 | −0.495 | −5.118 | −1.916 |
| Peptide 21 | 40.323 | −1.7 | 0.242 | −4.916 | −2.014 |
| Peptide 22 | 53.023 | −7.094 | 2 | −8.792 | −4.042 |
| Peptide 23 | 46.109 | −5.589 | 1.76 | −7.578 | −3.532 |
| Peptide 24 | 39.3 | −4.921 | 0.873 | −5.945 | −3.134 |
| Peptide 25 | 36.717 | −1.03 | −1.491 | −4.081 | −1.892 |
| Peptide 26 | 45.223 | −4.635 | 1.204 | −7.328 | −3.28 |
| Peptide 27 | 38.148 | −3.112 | 0.824 | −6.999 | −2.411 |
| Peptide 29 | 44.265 | −3.54 | 0.922 | −6.579 | −2.833 |
| Peptide 34 | 43.462 | −4.512 | 1.219 | −8.055 | −3.155 |
| Peptide 35 | 51.969 | −7.021 | 2 | −8.096 | −4.063 |
| Peptide 37 | 47.351 | −6.346 | 2 | −7.876 | −3.512 |
| Peptide 38 | 44.253 | −2.716 | −1.555 | −8.053 | −2.543 |
| Peptide 42 | 37.595 | −1.226 | −1.655 | −4.932 | −2.054 |
| Peptide 43 | 43.852 | −3.683 | 1.257 | −5.581 | −2.823 |
| Peptide 44 | 45.79 | −6.096 | 0.089 | −7.699 | −3.379 |
| Peptide 50 | 43.142 | −1.713 | −3.063 | −6.956 | −2.202 |
| Peptide 52 | 45.482 | −2.025 | −1.365 | −6.695 | −2.317 |
| Peptide 54 | 38.769 | −3.091 | 0.388 | −5.798 | −2.632 |
| Peptide 57 | 43.382 | 4.897 | 0.308 | −7.341 | −2.992 |
| Peptide 58 | 41.778 | −4.687 | 1.334 | −6.173 | −3.076 |
| MH3 | 67.848 | −9.533 | 2 | −13.707 | −4.705 |
| MH42 | 56.292 | −3.137 | −0.693 | −10.495 | −3.222 |
| MH43 | 60.745 | −5.618 | 1.171 | −11.56 | −4.048 |
| MH50 | 60.646 | −3.62 | −1.8 | −12.128 | −3.456 |

TABLE 10
Legend for the computed physicochemical properties disclosed in Tables 6 to 9

| Property | Description |
|---|---|
| #stats | Number of property or descriptor values that fall outside the 95% range of similar values for known drugs. |
| #rotor | Number of non trivial (not CX3), non-hindered (not alkene, amide, small ring) rotatable bonds. |
| mol_MW | Molecular weight (g/mol). |
| #rtvFG | Number of reactive functional groups. |
| SASA | Total solvent accessible area in square angstroms using a probe with 1.4 A radius. |
| FOSA | Hydrophobic component of the SASA (saturated carbon and attached hydrogen). |
| FISA | Hydrophilic component of the SASA (SASA on N, O, H on heeroatoms, carbonyl C). |
| PISA | π (carbon and attached hydrogen) component of the SASA |
| WPSA | Weakly polar component of the SASA (halogens, P, and S). 0.0-175.0 volume Total |
| | solvent-accessible volume in cubic angstroms using a probe with a 1.4 Å radius. |
| Volume | Total solvent-accessible volume in cubic angstroms using a probe with a 1.4 Å radius. |
| donorHB | Estimated number of hydrogen bonds that would be donated by the solute to water molecules in an aqueous solution. |
| accptHB | Estimated number of hydrogen bonds that would be accepted by the solute from water molecules in an aqueous solution. |
| glob | Globularity descriptor, , where r is the radius of a sphere with a volume equal to the molecular volume. |
| | Globularity is 1.0 for a spherical molecule. |
| QPpolrz | Predicted polarizability in cubic angstroms. |
| QPlogPC16 | Predicted hexadecane/gas partition coefficient. |
| QPlogPoct‡ | Predicted octanol/gas partition coefficient. |
| QPlogPw | Predicted water/gas partition coefficient. |
| GPlogPo/w | Predicted octanol/water partition coefficient. |
| QPlogS | Predicted aqueous solubility, log S. S in mol dm−3 is the concentration of the solute |
| | in a saturated solution that is in equilibrium with the crystalline solid. |
| QPlogBB | Predicted brain/blood partition coefficient. |
| #metab | Number of likely metabolic reactions |
| QPlogKhsa | Prediction of binding to human serum albumin. |
| PSA | Van der Waals surface area of polar nitrogen and oxygen atoms and carbonyl carbon atoms |
| QPlogHERG | Predicted IC50 value for blockage of HERG K+ channels. |
| #ringetoms | Number of atoms in a ring |
| #in34 | Number of atoms in 3- or 4-membered rings |
| #in56 | Number of atoms in 5- or 6-membered rings |
| #noncon | number of ring atoms not able to form conjugated aromatic systems (e.g. sp3 C). |
| #nonHatm | Number of heavy atoms (nonhydrogen atoms) |

TABLE 11
PEG amounts in LNPs (Mol %)

| | LNPm- | LNPm- | LNPm- | LNPm- | LNPm- | LNPm- |
|---|---|---|---|---|---|---|
| PEG | 0% | 0.15% | 0.3% | 0.6% | 0.9% | 1.2% |
| DMG-PEG | 1.5 | 1.35 | 1.2 | 0.9 | 0.6 | 0.3 |
| 2000 | | | | | | |
| DSPE-PEG | | 0.15 | 0.3 | 0.6 | 0.9 | 1.2 |
| (2000) | | | | | | |
| Maleimide | | | | | | |
| Total | 1.5 | 1.5 | 1.5 | 1.5 | 1.5 | 1.5 |

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

### VI. Embodiments

In certain embodiments, the present disclosure provides any one of embodiments 1 to 39 below:

1. A peptide according to Formula I

R¹—X₁—X₂—(X₃)ₙ—X₄—X₅—X₆—X₇—R² Formula I

wherein:
n is 0, 1, or 2;
X₁ is polar/hydrophilic or amphipathic/hydrophobic or non-polar/hydrophobic;
X₂ is non-polar/hydrophobic or polar/hydrophilic;
If present, each X₃ independently is non-polar/hydrophobic or polar/hydrophilic;
X₄ is non-polar/hydrophobic or polar/hydrophilic;
X₅ is polar/hydrophilic or non-polar/hydrophobic;
X₆ is non-polar/hydrophobic or amphipathic/hydrophobic or polar/hydrophilic;
X₇ is non-polar/hydrophobic or polar/hydrophilic/basic; and
R¹ is H, an amino acid residue, or a linker moiety;
R² is OH, an amino acid residue, or a linker moiety;
   and wherein the amino acid residues are defined as follows
Hydrophobic: Ala, Val, Gly, Ile, Leu, Phe, Pro, Trp, Tyr, Met, Cys;
Hydrophilic: Arg, Asn, Asp, Glu, Gln, Lys, Ser, Thr, His;
Non Polar: Ala, Gly, Ile, Leu, Phe, Val, Pro;
Amphipathic: Trp, Tyr, Met;
Polar: Ser, Thr, Asn, Gln, Arg, Lys, Asp, His, Cys, Glu;
Basic: Arg, Lys, His;
Acidic: Asp, Glu.

2. The peptide according to embodiment 1, wherein one or more of the following conditions apply:
a) the peptide contains one or more polar amino acid residues;
b) the peptide contains at least one hydrophobic amino acid residue;
c) the peptide contains 1-3 hydrophobic amino acid residues;
d) the peptide contains 1-5 non-polar amino acid residues;
e) the peptide does not contain an acid amino acid residue.

3. The peptide according to embodiment 1 or embodiment 2, wherein X₁ is non-polar/hydrophobic or Ser or Met.

4. The peptide according to any one of embodiments 1-3, wherein X₂ is non-polar/hydrophobic or Asn or Thr.

5. The peptide according to any one of embodiments 1-4, wherein n is 1 or 2 and each X₃ independently is non-polar/hydrophobic or Ser.

6. The peptide according to any one of embodiments 1-5, wherein X₄ is non-polar/hydrophobic or Ser.

7. The peptide according to any one of embodiments 1-6, wherein X₅ is non-polar/hydrophobic or polar/hydrophobic/basic, such as non-polar/hydrophobic or Arg or His.

8. The peptide according to any one of embodiments 1-7, wherein X₆ is non-polar/hydrophobic or polar/hydrophobic or Tyr.

9. The peptide according to any one of embodiments 1-8, wherein X₇ is non-polar/hydrophobic or Arg.

10. The peptide according to embodiment 1 or embodiment 2, wherein X₁ is polar/hydrophilic, X₂ is non-polar/hydrophobic, X₃ is non-polar/hydrophobic, X₄ is non-polar/hydrophobic, X₅ is polar/hydrophilic/basic, X₆ is non-polar/hydrophobic, and X₇ is non-polar/hydrophobic.

11. The peptide according to embodiment 1 or embodiment 2, wherein X₁ is polar/hydrophilic, X₂ is polar/hydrophilic, X₃ is non-polar/hydrophobic, X₄ is non-polar/hydrophobic, X₅ is non-polar/hydrophobic, X₆ is non-polar/hydrophobic, and X₇ is non-polar/hydrophobic.

12. The peptide according to embodiment 1 or embodiment 2, wherein X₁ is amphipatic/hydrophobic, X₂ is non-polar/hydrophobic, X₃ is non-polar/hydrophobic, X₄ is non-polar/hydrophobic, X₅ is non-polar/hydrophobic, X₆ is amphipatic/hydrophobic, and X₇ is polar/hydrophilic/basic.

13. The peptide according to embodiment 1 or embodiment 2, wherein X₁ is non-polar/hydrophobic, X₂ is polar/hydrophilic, X₃ is non-polar/hydrophobic, X₄ is non-polar/hydrophobic, X₅ is polar/hydrophilic/basic, X₆ is polar/hydrophilic, and X₇ is non-polar/hydrophobic.

14. The peptide according to embodiment 1 or embodiment 2, wherein X₁ is polar/hydrophilic/basic, X₂ is non-polar/hydrophobic, X₃ is polar/hydrophilic, X₄ is polar/hydrophilic, X₅ is non-polar/hydrophobic, X₆ is polar/hydrophilic, and X₇ is non-polar/hydrophobic.

15. The peptide according to embodiment 1, wherein:
X₁ is Ser, Met, Ala or His;
X₂ is Pro, Asn, Thr or Leu;
If present, each X₃ independently is Ala, Leu, Val, Gly, or Ser;
X₄ is Leu, Ala, Pro, or Ser;
X₅ is His, Ala, Val, Arg, or Leu;
X₆ is Phe, Tyr, Ser, or Thr;
X₇ is Leu, Pro, Arg, Val, or Pro; or a combination thereof.

16. The peptide if any one of embodiments 1-15, wherein n is 1.

17. The peptide of any one of embodiments 1-16, wherein R¹ is H, and R² is OH.

18. The peptide of embodiment 1, wherein the peptide is selected from:

Peptide 42:
(SEQ ID NO. 23)
Ser-Pro-Ala-Leu-His-Phe-Leu;
Peptide 43:
(SEQ ID No. 24)
Ser-Asn-Leu-Ala-Ala-Phe-Pro;
Peptide 50:
(SEQ ID No. 26)
Met-Pro-Val-Ala-Val-Tyr-Arg;
Peptide 57:
(SEQ ID No. 29)
Ala-Thr-Gly-Pro-Arg-Ser-Val;
or
Peptide 54:
(SEQ ID No. 28)
His-Leu-Ser-Ser-Leu-Thr-Pro.

19. The peptide of any one of embodiments 1-16, wherein at least one of R¹ and R² is a linker moiety.

20. The peptide of embodiment 19, wherein R¹ is H and R² is the linker moiety.

21. The peptide of any one of embodiments 19-20, wherein the linker moiety is a peptide sequence having from 2 to 7 amino acid residues.

22. The peptide of embodiment 21, wherein the linker moiety is a peptide sequence having 5 amino acid residues.

23. The peptide of embodiment 22, wherein the linker moiety has a sequence Gly-Gly-Gly-Ser (SEQ ID No. 35), Gly-Gly-Gly-Ser-Cys (SEQ ID No. 52) or Gly-Gly-Gly-Ser-Lys (SEQ ID No. 53).

24. The peptide of embodiment 22, wherein the peptide is selected from:

(SEQ ID No. 32)
Ser-Pro-Ala-Leu-His-Phe-Leu-Gly-Gly-Gly-Ser-Cys;
(SEQ ID No. 33)
Ser-Asn-Leu-Ala-Ala-Phe-Pro-Gly-Gly-Gly-Ser-Cys;
or
(SEQ ID No. 34)
Met-Pro-Val-Ala-Val-Tyr-Arg-Gly-Gly-Gly-Ser-Cys.

25. The peptide according to any one of embodiments 1-16, wherein each of R¹ and R² is an amino acid residue.

26. The peptide according to embodiment 25, wherein R¹ and R² are each Cys.

27. The peptide according to embodiment 26, wherein the peptide has a structure according to Formula III

Cys-X₁—X₂—(X₃)ₙ—X₄—X₅—X₆—X₇-Cys Formula III.

28. The peptide according to embodiment 25, wherein the peptide has a structure according to Formula IV

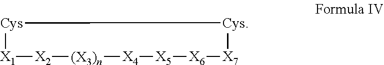

29. A composition comprising the peptide of any one of embodiments 1-28 and an additional agent.

30. The composition of embodiment 29, wherein the additional agent is a lipid nanoparticle or an imaging agent.

31. The composition of embodiment 30, wherein the imaging agent is a dye, fluorophore or radiotracer.

32. The composition of embodiment 30, wherein the lipid nanoparticle comprises or contains a therapeutic agent.

33. The composition of embodiment 32, wherein the therapeutic agent is a nucleic acid.

34. The composition of embodiment 33, wherein the nucleic acid comprises an antisense oligonucleotide (ASO), mRNA, siRNA, miRNA, or a combination thereof.

35. The composition of any one of embodiments 29-34, further comprising a pharmaceutically acceptable excipient.

36. A method, comprising administering to a subject a peptide according to any one of embodiments 1-28, or a composition according to any one of embodiments 29-35.

37. The method of embodiment 36, wherein administering to the subject comprises administering to an eye of the subject.

38. The method of embodiment 36 or embodiment 37, wherein administering comprises administering by injection.

39. The method of embodiment 36, comprising administering the peptide or the composition to an eye of the subject by intravitreal or subretinal injection.

### EQUIVALENTS AND SCOPE

In the claims and throughout, articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following claims.

We claim:

1. A peptide according to Formula I or V: 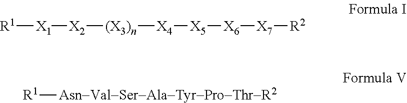
wherein:
n is 0, 1, or 2;
X₁ is a polar and hydrophilic amino acid, or amphipathic and hydrophobic amino acid, or non-polar and hydrophobic amino acid;
X₂ is a non-polar and hydrophobic amino acid or polar and hydrophilic amino acid;
If present, each X₃ independently is a non-polar and hydrophobic amino acid or polar and hydrophilic amino acid;
X₄ is a non-polar and hydrophobic amino acid or polar and hydrophilic amino acid;
X₅ is a polar and hydrophilic amino acid or non-polar and hydrophobic amino acid;
X₆ is a non-polar and hydrophobic amino acid, or amphipathic and hydrophobic amino acid, or polar and hydrophilic amino acid;
X₇ is a non-polar and hydrophobic amino acid or polar and hydrophilic and basic amino acid; and
R¹ is H, an amino acid, a linker moiety, or a nitrogen protecting group;
R² is OH, an amino acid, a linker moiety, or O(an oxygen protecting group);
and wherein the amino acids are defined as follows:
each hydrophobic amino acid is independently: Ala, Val, Gly, Ile, Leu, Phe, Pro, Trp, Tyr, Met, or Cys;
each hydrophilic amino acid is independently: Arg, Asn, Asp, Glu, Gln, Lys, Ser, Thr, or His;
each non-polar amino acid is independently: Ala, Gly, Ile, Leu, Phe, Val, or Pro;
each amphipathic amino acid is independently: Trp, Tyr, or Met;
each polar amino acid is independently: Ser, Thr, Asn, Gln, Arg, Lys, Asp, His, Cys, or Glu;
each basic amino acid is independently: Arg, Lys, or His; and
each acidic amino acid is independently: Asp or Glu.


SEQUENCE LISTING (98 sequences)

SEQ ID NO: 1 - protein, 7 aa, synthetic construct
  REGION 1..7: note = Synthetic amino acid sequence
HILNWPT

SEQ ID NO: 2 - protein, 7 aa, synthetic construct
  REGION 1..7: note = Synthetic amino acid sequence
DGPPRKP

SEQ ID NO: 3 - protein, 7 aa, synthetic construct
  REGION 1..7: note = Synthetic amino acid sequence
HLSSIPG

SEQ ID NO: 4 - protein, 7 aa, synthetic construct
  REGION 1..7: note = Synthetic amino acid sequence
FPHILAP

SEQ ID NO: 5 - protein, 7 aa, synthetic construct
  REGION 1..7: note = Synthetic amino acid sequence
HVSPLNW

SEQ ID NO: 6 - protein, 7 aa, synthetic construct
  REGION 1..7: note = Synthetic amino acid sequence
VFPMPPM

SEQ ID NO: 7 - protein, 7 aa, synthetic construct
  REGION 1..7: note = Synthetic amino acid sequence
NVDNYPH

SEQ ID NO: 8 - protein, 7 aa, synthetic construct
  REGION 1..7: note = Synthetic amino acid sequence
YPQGPFP

SEQ ID NO: 9 - protein, 7 aa, synthetic construct
  REGION 1..7: note = Synthetic amino acid sequence
AFAPFKT

SEQ ID NO: 10 - protein, 7 aa, synthetic construct
  REGION 1..7: note = Synthetic amino acid sequence
YFPAFTS

SEQ ID NO: 11 - protein, 7 aa, synthetic construct
  REGION 1..7: note = Synthetic amino acid sequence
IYAKAPI

SEQ ID NO: 12 - protein, 7 aa, synthetic construct
  REGION 1..7: note = Synthetic amino acid sequence
SQTNAIH

SEQ ID NO: 13 - protein, 7 aa, synthetic construct
  REGION 1..7: note = Synthetic amino acid sequence
LSINNVT

SEQ ID NO: 14 - protein, 7 aa, synthetic construct
  REGION 1..7: note = Synthetic amino acid sequence
SSPAAHT

SEQ ID NO: 15 - protein, 7 aa, synthetic construct
  REGION 1..7: note = Synthetic amino acid sequence
WGPPTLI

SEQ ID NO: 16 - protein, 7 aa, synthetic construct
  REGION 1..7: note = Synthetic amino acid sequence
SIAHNTM

SEQ ID NO: 17 - protein, 7 aa, synthetic construct
  REGION 1..7: note = Synthetic amino acid sequence
TVKTLTT

SEQ ID NO: 18 - protein, 7 aa, synthetic construct
  REGION 1..7: note = Synthetic amino acid sequence
FIPNGSM

SEQ ID NO: 19 - protein, 7 aa, synthetic construct
  REGION 1..7: note = Synthetic amino acid sequence
SLTNSSF

SEQ ID NO: 20 - protein, 7 aa, synthetic construct
  REGION 1..7: note = Synthetic amino acid sequence
INLNNVT

SEQ ID NO: 21 - protein, 7 aa, synthetic construct
  REGION 1..7: note = Synthetic amino acid sequence
ATKSYNT

SEQ ID NO: 22 - protein, 7 aa, synthetic construct
  REGION 1..7: note = Synthetic amino acid sequence
TSMLPYR

SEQ ID NO: 23 - protein, 7 aa, synthetic construct
  REGION 1..7: note = Synthetic amino acid sequence
SPALHFL

SEQ ID NO: 24 - protein, 7 aa, synthetic construct
  REGION 1..7: note = Synthetic amino acid sequence
SNLAAFP

SEQ ID NO: 25 - protein, 7 aa, synthetic construct
  REGION 1..7: note = Synthetic amino acid sequence
DVATNMH

SEQ ID NO: 26 - protein, 7 aa, synthetic construct
  REGION 1..7: note = Synthetic amino acid sequence
MPVAVYR

SEQ ID NO: 27 - protein, 7 aa, synthetic construct
  REGION 1..7: note = Synthetic amino acid sequence
LAFHRMP

SEQ ID NO: 28 - protein, 7 aa, synthetic construct
  REGION 1..7: note = Synthetic amino acid sequence
HLSSLTP

SEQ ID NO: 29 - protein, 7 aa, synthetic construct
  REGION 1..7: note = Synthetic amino acid sequence
ATGPRSV

SEQ ID NO: 30 - protein, 7 aa, synthetic construct
  REGION 1..7: note = Synthetic amino acid sequence
NVSAYPT

SEQ ID NO: 31 - protein, 12 aa, synthetic construct
  REGION 1..12: note = Synthetic amino acid sequence
DGPPRKPGGGSC

SEQ ID NO: 32 - protein, 12 aa, synthetic construct
  REGION 1..12: note = Synthetic amino acid sequence
SPALHFLGGGSC

SEQ ID NO: 33 - protein, 12 aa, synthetic construct
  REGION 1..12: note = Synthetic amino acid sequence
SNLAAFPGGGSC

SEQ ID NO: 34 - protein, 12 aa, synthetic construct
  REGION 1..12: note = Synthetic amino acid sequence
MPVAVYRGGGSC

SEQ ID NO: 35 - protein, 4 aa, synthetic construct
  REGION 1..4: note = Synthetic amino acid sequence
GGGS

SEQ ID NO: 36 - DNA, 20 bp, synthetic construct
  misc_feature 1..20: note = Synthetic nucleic acid sequence
  source 1..20: mol_type = other DNA
ccctcatagttagcgtaacg

SEQ ID NO: 37 - protein, 7 aa, synthetic construct
  REGION 1..7: note = Synthetic amino acid sequence
  SITE 4: note = MISC_FEATURE - X is Asn or His
  SITE 7: note = MISC_FEATURE - X is Met or Thr
SIAXNTX

SEQ ID NO: 38 - protein, 7 aa, synthetic construct
  REGION 1..7: note = Synthetic amino acid sequence
YHQTVQC

SEQ ID NO: 39 - protein, 7 aa, synthetic construct
  REGION 1..7: note = Synthetic amino acid sequence
QLSTADV

SEQ ID NO: 40 - protein, 7 aa, synthetic construct
  REGION 1..7: note = Synthetic amino acid sequence
NPRFYPS

SEQ ID NO: 41 - protein, 7 aa, synthetic construct
  REGION 1..7: note = Synthetic amino acid sequence
ESCHQFP

SEQ ID NO: 42 - protein, 7 aa, synthetic construct
  REGION 1..7: note = Synthetic amino acid sequence
QTPATAG

SEQ ID NO: 43 - protein, 7 aa, synthetic construct
  REGION 1..7: note = Synthetic amino acid sequence
TSPSYSM

SEQ ID NO: 44 - protein, 7 aa, synthetic construct
  REGION 1..7: note = Synthetic amino acid sequence
YSRDSAA

SEQ ID NO: 45 - protein, 7 aa, synthetic construct
  REGION 1..7: note = Synthetic amino acid sequence
HMTVSVF

SEQ ID NO: 46 - protein, 7 aa, synthetic construct
  REGION 1..7: note = Synthetic amino acid sequence
CPRMPSV

SEQ ID NO: 47 - protein, 7 aa, synthetic construct
  REGION 1..7: note = Synthetic amino acid sequence
YHNMLPP

SEQ ID NO: 48 - protein, 7 aa, synthetic construct
  REGION 1..7: note = Synthetic amino acid sequence
SYAPHIA

SEQ ID NO: 49 - protein, 7 aa, synthetic construct
  REGION 1..7: note = Synthetic amino acid sequence
TVRYVEP

SEQ ID NO: 50 - protein, 7 aa, synthetic construct
  REGION 1..7: note = Synthetic amino acid sequence
RLGLEGP

SEQ ID NO: 51 - protein, 7 aa, synthetic construct
  REGION 1..7: note = Synthetic amino acid sequence
NLLSHPQ

SEQ ID NO: 52 - protein, 5 aa, synthetic construct
  REGION 1..5: note = Synthetic amino acid sequence
GGGSC

SEQ ID NO: 53 - protein, 5 aa, synthetic construct
  REGION 1..5: note = Synthetic amino acid sequence
GGGCK

SEQ ID NO: 54 - protein, 12 aa, synthetic construct
LAFHRMPGGGSC

SEQ ID NO: 55 - protein, 12 aa, synthetic construct
HLSSLTPGGGSC

SEQ ID NO: 56 - protein, 12 aa, synthetic construct
ATGPRSVGGGSC

SEQ ID NO: 57 - protein, 7 aa, synthetic construct
  VARIANT 1: note = X can be Ala, Asp, Glu, Phe, Gly, His, Ile, Lys, Leu, Met, Asn, Pro, Gln, Arg, Ser, Thr, Val, Trp, or Tyr
XPALHFL

SEQ ID NO: 58 - protein, 7 aa, synthetic construct
  VARIANT 2: note = X can be Ala, Asp, Glu, Phe, Gly, His, Ile, Lys, Leu, Asn, Pro, Gln, Arg, Ser, Thr, or Val
SXALHFL

SEQ ID NO: 59 - protein, 7 aa, synthetic construct
  VARIANT 3: note = X can be Ala, Asp, Glu, Phe, Gly, His, Ile, Lys, Leu, Asn, Pro, Gln, Arg, Ser, Thr, or Val
SPXLHFL

SEQ ID NO: 60 - protein, 7 aa, synthetic construct
  VARIANT 4: note = X can be Ala, Asp, Glu, Phe, Gly, His, Ile, Lys, Leu, Asn, Pro, Gln, Arg, Ser, Thr, or Val
SPAXHFL

SEQ ID NO: 61 - protein, 7 aa, synthetic construct
  VARIANT 5: note = X can be Ala, Asp, Glu, Phe, Gly, His, Ile, Lys, Leu, Asn, Pro, Gln, Arg, Ser, Thr, or Val
SPALXFL

SEQ ID NO: 62 - protein, 7 aa, synthetic construct
  VARIANT 6: note = X can be Ala, Asp, Glu, Phe, Gly, His, Ile, Lys, Leu, Met, Asn, Pro, Gln, Arg, Ser, Thr, Val, Trp, or Tyr
SPALHXL

SEQ ID NO: 63 - protein, 7 aa, synthetic construct
  VARIANT 7: note = X can be Ala, Phe, Gly, His, Ile, Lys, Leu, Pro, Arg, or Val
SPALHFX

SEQ ID NO: 64 - protein, 7 aa, synthetic construct
  VARIANT 1: note = X can be Ala, Asp, Glu, Phe, Gly, His, Ile, Lys, Leu, Met, Asn, Pro, Gln, Arg, Ser, Thr, Val, Trp, or Tyr
XNLAAFP

SEQ ID NO: 65 - protein, 7 aa, synthetic construct
  VARIANT 2: note = X can be Ala, Asp, Glu, Phe, Gly, His, Ile, Lys, Leu, Asn, Pro, Gln, Arg, Ser, Thr, or Val
SXLAAFP

SEQ ID NO: 66 - protein, 7 aa, synthetic construct
  VARIANT 3: note = X can be Ala, Asp, Glu, Phe, Gly, His, Ile, Lys, Leu, Asn, Pro, Gln, Arg, Ser, Thr, or Val
SNXAAFP

SEQ ID NO: 67 - protein, 7 aa, synthetic construct
  VARIANT 4: note = X can be Ala, Asp, Glu, Phe, Gly, His, Ile, Lys, Leu, Asn, Pro, Gln, Arg, Ser, Thr, or Val
SNLXAFP

SEQ ID NO: 68 - protein, 7 aa, synthetic construct
  VARIANT 5: note = X can be Ala, Asp, Glu, Phe, Gly, His, Ile, Lys, Leu, Asn, Pro, Gln, Arg, Ser, Thr, or Val
SNLAXFP

SEQ ID NO: 69 - protein, 7 aa, synthetic construct
  VARIANT 6: note = X can be Ala, Asp, Glu, Phe, Gly, His, Ile, Lys, Leu, Met, Asn, Pro, Gln, Arg, Ser, Thr, Val, Trp, or Tyr
SNLAAXP

SEQ ID NO: 70 - protein, 7 aa, synthetic construct
  VARIANT 7: note = X can be Ala, Phe, Gly, His, Ile, Lys, Leu, Pro, Arg, or Val
SNLAAFX

SEQ ID NO: 71 - protein, 7 aa, synthetic construct
  VARIANT 1: note = Ala, Asp, Glu, Phe, Gly, His, Ile, Lys, Leu, Met, Asn, Pro, Gln, Arg, Ser, Thr, Val, Trp, or Tyr
XPVAVYR

SEQ ID NO: 72 - protein, 7 aa, synthetic construct
  VARIANT 2: note = X can be Ala, Asp, Glu, Phe, Gly, His, Ile, Lys, Leu, Asn, Pro, Gln, Arg, Ser, Thr, or Val
MXVAVYR

SEQ ID NO: 73 - protein, 7 aa, synthetic construct
  VARIANT 3: note = X can be Ala, Asp, Glu, Phe, Gly, His, Ile, Lys, Leu, Asn, Pro, Gln, Arg, Ser, Thr, or Val
MPXAVYR

SEQ ID NO: 74 - protein, 7 aa, synthetic construct
  VARIANT 4: note = X can be Ala, Asp, Glu, Phe, Gly, His, Ile, Lys, Leu, Asn, Pro, Gln, Arg, Ser, Thr, or Val
MPVXVYR

SEQ ID NO: 75 - protein, 7 aa, synthetic construct
  VARIANT 5: note = X can be Ala, Asp, Glu, Phe, Gly, His, Ile, Lys, Leu, Asn, Pro, Gln, Arg, Ser, Thr, or Val
MPVAXYR

SEQ ID NO: 76 - protein, 7 aa, synthetic construct
  VARIANT 6: note = X can be Ala, Asp, Glu, Phe, Gly, His, Ile, Lys, Leu, Met, Asn, Pro, Gln, Arg, Ser, Thr, Val, Trp, or Tyr
MPVAVXR

SEQ ID NO: 77 - protein, 7 aa, synthetic construct
  VARIANT 7: note = X can be Ala, Phe, Gly, His, Ile, Lys, Leu, Pro, Arg, or Val
MPVAVYX

SEQ ID NO: 78 - protein, 7 aa, synthetic construct
  VARIANT 1: note = X can be Ala, Asp, Glu, Phe, Gly, His, Ile, Lys, Leu, Met, Asn, Pro, Gln, Arg, Ser, Thr, Val, Trp, or Tyr
XAFHRMP

SEQ ID NO: 79 - protein, 7 aa, synthetic construct
  VARIANT 2: note = X can be Ala, Asp, Glu, Phe, Gly, His, Ile, Lys, Leu, Asn, Pro, Gln, Arg, Ser, Thr, or Val
LXFHRMP

SEQ ID NO: 80 - protein, 7 aa, synthetic construct
  VARIANT 3: note = X can be Ala, Asp, Glu, Phe, Gly, His, Ile, Lys, Leu, Asn, Pro, Gln, Arg, Ser, Thr, or Val
LAXHRMP

SEQ ID NO: 81 - protein, 7 aa, synthetic construct
  VARIANT 4: note = X can be Ala, Asp, Glu, Phe, Gly, His, Ile, Lys, Leu, Asn, Pro, Gln, Arg, Ser, Thr, or Val
LAFXRMP

SEQ ID NO: 82 - protein, 7 aa, synthetic construct
  VARIANT 5: note = X can be Ala, Asp, Glu, Phe, Gly, His, Ile, Lys, Leu, Asn, Pro, Gln, Arg, Ser, Thr, or Val
LAFHXMP

SEQ ID NO: 83 - protein, 7 aa, synthetic construct
  VARIANT 6: note = X can be Ala, Asp, Glu, Phe, Gly, His, Ile, Lys, Leu, Met, Asn, Pro, Gln, Arg, Ser, Thr, Val, Trp, or Tyr
LAFHRXP

SEQ ID NO: 84 - protein, 7 aa, synthetic construct
  VARIANT 7: note = X can be Ala, Phe, Gly, His, Ile, Lys, Leu, Pro, Arg, or Val
LAFHRMX

SEQ ID NO: 85 - protein, 7 aa, synthetic construct
  VARIANT 1: note = X can be Ala, Asp, Glu, Phe, Gly, His, Ile, Lys, Leu, Met, Asn, Pro, Gln, Arg, Ser, Thr, Val, Trp, or Tyr
XLSSLTP

SEQ ID NO: 86 - protein, 7 aa, synthetic construct
  VARIANT 2: note = X can be Ala, Asp, Glu, Phe, Gly, His, Ile, Lys, Leu, Asn, Pro, Gln, Arg, Ser, Thr, or Val
HXSSLTP

SEQ ID NO: 87 - protein, 7 aa, synthetic construct
  VARIANT 3: note = X can be Ala, Asp, Glu, Phe, Gly, His, Ile, Lys, Leu, Asn, Pro, Gln, Arg, Ser, Thr, or Val
HLXSLTP

SEQ ID NO: 88 - protein, 7 aa, synthetic construct
  VARIANT 4: note = X can be Ala, Asp, Glu, Phe, Gly, His, Ile, Lys, Leu, Asn, Pro, Gln, Arg, Ser, Thr, or Val
HLSXLTP

SEQ ID NO: 89 - protein, 7 aa, synthetic construct
  VARIANT 5: note = X can be Ala, Asp, Glu, Phe, Gly, His, Ile, Lys, Leu, Asn, Pro, Gln, Arg, Ser, Thr, or Val
HLSSXTP

SEQ ID NO: 90 - protein, 7 aa, synthetic construct
  VARIANT 6: note = X can be Ala, Asp, Glu, Phe, Gly, His, Ile, Lys, Leu, Met, Asn, Pro, Gln, Arg, Ser, Thr, Val, Trp, or Tyr
HLSSLXP

SEQ ID NO: 91 - protein, 7 aa, synthetic construct
  VARIANT 7: note = X can be Ala, Phe, Gly, His, Ile, Lys, Leu, Pro, Arg, or Val
HLSSLTX

SEQ ID NO: 92 - protein, 7 aa, synthetic construct
  VARIANT 1: note = X can be Ala, Asp, Glu, Phe, Gly, His, Ile, Lys, Leu, Met, Asn, Pro, Gln, Arg, Ser, Thr, Val, Trp, or Tyr
XTGPRSV

SEQ ID NO: 93 - protein, 7 aa, synthetic construct
  VARIANT 2: note = X can be Ala, Asp, Glu, Phe, Gly, His, Ile, Lys, Leu, Asn, Pro, Gln, Arg, Ser, Thr, or Val
AXGPRSV

SEQ ID NO: 94 - protein, 7 aa, synthetic construct
  VARIANT 3: note = X can be Ala, Asp, Glu, Phe, Gly, His, Ile, Lys, Leu, Asn, Pro, Gln, Arg, Ser, Thr, or Val
ATXPRSV

SEQ ID NO: 95 - protein, 7 aa, synthetic construct
  VARIANT 4: note = X can be Ala, Asp, Glu, Phe, Gly, His, Ile, Lys, Leu, Asn, Pro, Gln, Arg, Ser, Thr, or Val
ATGXRSV

SEQ ID NO: 96 - protein, 7 aa, synthetic construct
  VARIANT 5: note = X can be Ala, Asp, Glu, Phe, Gly, His, Ile, Lys, Leu, Asn, Pro, Gln, Arg, Ser, Thr, or Val
ATGPXSV

SEQ ID NO: 97 - protein, 7 aa, synthetic construct
  VARIANT 6: note = X can be Ala, Asp, Glu, Phe, Gly, His, Ile, Lys, Leu, Met, Asn, Pro, Gln, Arg, Ser, Thr, Val, Trp, or Tyr
ATGPRXV

SEQ ID NO: 98 - protein, 7 aa, synthetic construct
  VARIANT 7: note = X can be Ala, Phe, Gly, His, Ile, Lys, Leu, Pro, Arg, or Val
ATGPRSX